(19) United States
(12) Patent Application Publication
(10) Pub. No.: US 2022/0298516 A1
(43) Pub. Date: Sep. 22, 2022

(54) COMPOSITIONS AND METHODS FOR DELIVERY OF NUCLEIC ACIDS

(71) Applicant: ModernaTX, Inc., Cambridge, MA (US)

(72) Inventors: Aaron LARSEN, Cambridge, MA (US); Jennifer NELSON, Brookline, MA (US); Melissa MOORE, Cambridge, MA (US)

(21) Appl. No.: 17/276,983

(22) PCT Filed: Sep. 20, 2019
(86) PCT No.: PCT/US2019/052058

§ 371 Date: Mar. 17, 2021

Related U.S. Application Data
(60) Provisional application No. 62/734,082, filed on Sep. 20, 2018.

(51) Int. Cl. C12N 15/67; C12N 15/88; A61K 47/00; A61K 31/7088; C12N 15/11

(52) U.S. Cl. CPC C12N 15/67; C12N 15/88; A61K 47/00; A61K 31/7088; C12N 15/111; C12N 2310/152; C12N 2310/531; C12N 2310/51

(57) ABSTRACT

The present disclosure relates to methods and compositions for modulating protein expression. In particular, the invention features methods and compositions for increasing protein expression in a cell by delivering to the cell a composition including an mRNA encoding a polypeptide and one or more oligonucleotides, wherein each of the one or more oligonucleotides includes a region of linked nucleotides complimentary to a portion of the sequence of the mRNA. The methods and compositions described herein may be used to modulate gene expression (e.g., increase gene expression), to increase the stability of the mRNA, to decrease the immunogenicity of the mRNA, to enable selective expression (e.g., in a target cell or tissue) of the mRNA, and/or to enable the delivery of two or more mRNAs in a stoichiometric ratio.

### SEQUENCE LISTING

The instant application contains a sequence listing which has been submitted electronically in ASCII format and is hereby incorporated by reference in its entirety. Said ASCII copy, created on Dec. 20, 2021, is named “50858-100002_Sequence_Listing_12_20_21_ST25” and is 5,131 bytes in size.

### BACKGROUND OF THE INVENTION

Altering the expression levels of proteins associated with disease is desirable for a wide range of therapeutic applications. Methods for inhibiting the expression of genes are known in the art and include, for example, antisense oligonucleotides, RNAi, and miRNA mediated approaches. Such methods may involve blocking translation of mRNAs or causing degradation of target RNAs. However, limited approaches are available for increasing the expression of genes.

Multiple problems associated with prior methodologies of increasing gene expression limit their therapeutic applications. For example, heterologous DNA introduced into a cell can be inherited by daughter cells (whether or not the heterologous DNA has integrated into the chromosome) or by offspring. Introduced DNA can integrate into host cell genomic DNA at some frequency, resulting in alterations and/or damage to the host cell genomic DNA. Further, it is difficult to obtain expression of heterologous nucleic acids in cells. Finally, the delivery of nucleic acids to a cell in a subject, such as a human subject, is limited by low stability, low selectivity for the target cell, and high immunogenicity.

Accordingly, there is a need in the art for new methodologies to selectively increase gene expression.

### SUMMARY OF THE INVENTION

The present disclosure provides compositions including one or more mRNAs and one or more oligonucleotides, wherein each oligonucleotide includes a region of linked nucleotides complimentary to a portion of the sequence of the mRNA. A composition described herein may be used to modulate gene expression (e.g., increase gene expression) of the one or more mRNAs, for example, by administering the composition to a cell or a subject. A composition described herein may increase the stability of the one or more mRNAs (e.g., by decreasing endonuclease or exonuclease degradation). A composition described herein may decrease the immunogenicity of (e.g., lower the innate immune response associated with) the one or more mRNAs. A composition described herein may enable selective expression (e.g., in a target cell or tissue) of the one or more mRNAs. A composition described herein may also enable the delivery of two or more mRNAs to a cell in a stoichiometric ratio. The present disclosure also provides methods of making and using the aforementioned compositions.

In one aspect, the invention features a composition including: (a) an mRNA encoding a polypeptide including: (i) a 5′-cap structure; (ii) a 5′-untranslated region (5′-UTR); (iii) an open reading frame encoding the polypeptide; (iv) a 3′-untranslated region (3′-UTR); and (v) a poly-A region; and (b) three or more oligonucleotides (e.g., 3, 4, 5, 6, 7, 8, 9, 10, 15, 20, 25, 30, 35, 40, 45, or 50 or more oligonucleotides), wherein each oligonucleotide includes a region of linked nucleotides complimentary to a different portion of the sequence of the mRNA.

In some embodiments, the composition includes at least three and no more than ten oligonucleotides. In some embodiments, the composition includes at least ten and no more than fifty oligonucleotides. In some embodiments, the composition includes 3 to 5, 5 to 10, 10 to 15, 15 to 20, 20 to 25, 25 to 30, 30 to 35, 35 to 40, 40 to 45, or 45 to 50 oligonucleotides.

In some embodiments, the oligonucleotides collectively include regions of linked nucleotides complementary to 10% or more (e.g., 20% or more, 30% or more, 40% or more, 50% or more, 60% or more, 70% or more, 80% or more, 90% or more, 95% or more, 99% or more, or 100%) of the sequence of the mRNA.

In some embodiments, each oligonucleotide includes between 6 and 100 nucleotides (e.g., 6 to 10, 6 to 20, 6 to 30, 6 to 40, 6 to 50, 6 to 60, 6 to 70, 6 to 80, 6 to 90, 10 to 20, 10 to 30 10 to 40, 10 to 50, 10 to 60, 10 to 70, 10 to 80, 10 to 90, 10 to 100, 20 to 30, 20 to 40, or 20 to 50). In some embodiments, each oligonucleotide includes at least 6, 8, 10, 12, 14, 16, 18, 20, 25, 30, 35, 40, 35, or 50 nucleotides.

In some embodiments, each oligonucleotide includes a region of linked nucleotides complementary to a portion of a sequence of the mRNA, wherein the region of linked nucleotides is at least 5 nucleotides (e.g., a least 6, 7, 8, 9, 10, 12, 15, 18, 20, 25, 30, 35, 40, 45, or 50 nucleotides).

In some embodiments, each oligonucleotide includes at least one alternative internucleoside linkage, nucleobase analog, sugar analog, or nucleoside analog as described herein. In some embodiments, each oligonucleotide includes at least one 2′-OMe nucleotide, 2′-MOE nucleotide, 2′-F nucleotide, 2′-NH₂ nucleotide, FANA nucleotide, LNA nucleotide, 4′-S nucleotide, TNA nucleotide, or PNA nucleotide. In some embodiments, each oligonucleotide includes at least one 2′-OMe nucleotide. In some embodiments, each oligonucleotide consists of 2′-OMe nucleotides.

In some embodiments, at least one oligonucleotide includes a region of linked nucleotides (e.g., a region of 5 or more, 10 or more, 15 or more, 20 or more, 25 or more, 30 or more, 35 or more, 40 or more, 45 or more, or 50 or more linked nucleotides) complementary to a portion of the sequence of the 5′-UTR or the 3′-UTR. In some embodiments, at least one oligonucleotide includes a region of linked nucleotides (e.g., a region of 5 or more, 10 or more, 15 or more, 20 or more, 25 or more, 30 or more, 35 or more, 40 or more, 45 or more, or 50 or more linked nucleotides) complementary to a portion of the sequence of the start codon.

In some embodiments, the mRNA is hybridized to each of the oligonucleotides.

In some embodiments, at least one of the oligonucleotides is conjugated to a moiety selected from a sterol, a polyethylene glycol, a polylactic acid, a sugar, a toll-like receptor antagonist, or an endosomal escape peptide. In some embodiments, each of the oligonucleotides is conjugated to a moiety selected from a sterol, a polyethylene glycol (PEG), a polylactic acid, a sugar, a toll-like receptor antagonist, or an endosomal escape peptide. In some embodiments, the moiety is a sterol (e.g., cholesterol).

In some embodiments, the moiety is conjugated to the oligonucleotide via a linker, such as any of the linkers described herein (e.g., a PEG linker).

In some embodiments, the moiety is conjugated to the 5′-terminus of the oligonucleotide, the 3′-terminus of the oligonucleotide, or an internal nucleotide via a linkage to the Hoogsteen face of a nucleobase.

In another aspect, the invention features a composition including: (a) an mRNA encoding a polypeptide including: (i) a 5′-cap structure; (ii) a 5′-UTR; (iii) an open reading frame encoding the polypeptide; (iv) a 3′-UTR; and (v) a poly-A region; and (b) a conjugate including an oligonucleotide including a region of linked nucleotides complimentary to a portion of the sequence of the mRNA and at least one sterol moiety (e.g., cholesterol).

In some embodiments, the oligonucleotide includes between 6 and 100 nucleotides (e.g., 6 to 10, 6 to 20, 6 to 30, 6 to 40, 6 to 50, 6 to 60, 6 to 70, 6 to 80, 6 to 90, 10 to 20, 10 to 30 10 to 40, 10 to 50, 10 to 60, 10 to 70, 10 to 80, 10 to 90, 10 to 100, 20 to 30, 20 to 40, or 20 to 50). In some embodiments, each oligonucleotide includes at least 6, 8, 10, 12, 14, 16, 18, 20, 25, 30, 35, 40, 35, or 50 nucleotides.

In some embodiments, the region of linked nucleotides complementary to a portion of a sequence of the mRNA is at least 5 nucleotides (e.g., a least 6, 7, 8, 9, 10, 12, 15, 18, 20, 25, 30, 35, 40, 45, or 50 nucleotides).

In some embodiments, each oligonucleotide includes at least one alternative internucleoside linkage, nucleobase analog, sugar analog, or nucleoside analog as described herein. In some embodiments, the oligonucleotide includes at least one 2′-OMe nucleotide, 2′-MOE nucleotide, 2′-F nucleotide, 2′-NH₂ nucleotide, FANA nucleotide, LNA nucleotide, 4′-S nucleotide, TNA nucleotide, or PNA nucleotide. In some embodiments, the oligonucleotide includes at least one 2′-OMe nucleotide. In some embodiments, the oligonucleotide consists of 2′-OMe nucleotides.

In some embodiments, the oligonucleotide includes a region of linked nucleotides (e.g., a region of 5 or more, 10 or more, 15 or more, 20 or more, 25 or more, 30 or more, 35 or more, 40 or more, 45 or more, or 50 or more linked nucleotides) complementary to a portion of the sequence of the 5′-UTR, the 3′-UTR, the open reading frame, the start codon, the stop codon, or poly-A region of the mRNA. In some embodiments, the oligonucleotide includes a region of linked nucleotides complementary to a portion of the sequence of the 5′-UTR or the 3′-UTR. In some embodiments, the oligonucleotide includes a region of linked nucleotides complementary to a portion of the sequence of the start codon.

In some embodiments, the mRNA and conjugate are hybridized.

In some embodiments, the sterol is selected from adosterol, agosterol A, atheronals, avenasterol, azacosterol, blazein, a blood lipid, cerevisterol, cholesterol, cholesterol sulfate, colestolone, cycloartenol, daucosterol, 7-dehydrocholesterol, 5-dehydroepisterol, 7-dehydrositosterol, 20α,22R-dihydroxycholesterol, dinosterol, epibrassicasterol, episterol, ergosterol, ergosterol, fecosterol, fucosterol, fungisterol, ganoderenic acid, ganoderic acid, ganoderiol, ganodermadiol, 7α-hydroxycholesterol, 22R-hydroxycholesterol, 27-hydroxycholesterol, inotodiol, lanosterol, lathosterol, lichesterol, lucidadiol, lumisterol, oxycholesterol, oxysterol, parkeol, saringosterol, spinasterol, sterol ester, trametenolic acid, zhankuic acid, or zymosterol. In some embodiments, the sterol is cholesterol.

In some embodiment, the conjugate includes 2 or more sterols, 3 or more sterols, 4 or more sterols, or 5 or more sterols.

In some embodiments, the sterol is conjugated to a nucleotide by way of a linker. In some embodiments, the linker includes a polyethylene glycol linker (a PEG linker).

In some embodiments, the sterol is conjugated to the 5′-terminus of the oligonucleotide, the 3′-terminus of the oligonucleotide, or an internal nucleotide via a linkage to the Hoogsteen face of a nucleobase.

In some embodiments, the composition further includes: (c) a second conjugate including a region of linked nucleotides complimentary to at least a second portion of the sequence of the mRNA, and optionally at least one sterol moiety.

In some embodiments, upon administration to a cell, the composition induces a lower innate immune response compared to the mRNA alone. In some embodiments, the composition induces an innate immune response that is at least 10%, 20%, 30%, 40%, 50%, 60%, 70%, 80%, 90%, 100%, 150%, 200%, 500%, or 1000% lower than the innate immune response induced when the mRNA is administered alone. In some embodiments, the composition induces an innate immune response that is at least 2 times, 3 times, 4 times, 5 times, 10 times, 20 times, 50 times, or 100 times lower than the innate immune response induced when the mRNA is administered alone.

In another aspect the invention features a method of decreasing the innate immune response induced by an mRNA upon administration to a cell (e.g., a cell of a subject, such as a human subject), wherein the method includes hybridizing the mRNA to a conjugate including an oligonucleotide including a region of linked nucleotides complimentary to a portion of the sequence of the mRNA and at least one sterol moiety.

In another aspect, the invention features a composition including: (a) an mRNA encoding a polypeptide including: (i) a 5′-cap structure; (ii) a 5′-UTR; (iii) an open reading frame encoding the polypeptide; (iv) a 3′-UTR; and (v) a poly-A region; and (b) a conjugate including the structure: A-L-B; wherein A is a first oligonucleotide, L is a linker including a cleavage site, and B is a second oligonucleotide, wherein A and B each include a region of linked nucleotides complimentary to a different portion of the sequence of the mRNA.

In some embodiments, upon administration to a cell (e.g., a cell of a subject, such as a human subject), the composition decreases expression of the mRNA compared to administration of the mRNA alone. In some embodiments, the composition results in an expression level of the encoded polypeptide that is at least 10%, 20%, 30%, 40%, 50%, 60%, 70%, 80%, 90%, 100%, 150%, 200%, 500%, or 1000% less than the expression level of the encoded polypeptide when the mRNA is administered alone. In some embodiments, the composition results in an expression level of the encoded polypeptide that is at least 2 times, 3 times, 4 times, 5 times, 10 times, 20 times, 50 times, or 100 times less than the expression level of the encoded polypeptide when the mRNA is administered alone.

In some embodiments, A and B each, independently, include between 6 and 100 nucleotides (e.g., 6 to 10, 6 to 20, 6 to 30, 6 to 40, 6 to 50, 6 to 60, 6 to 70, 6 to 80, 6 to 90, 10 to 20, 10 to 30 10 to 40, 10 to 50, 10 to 60, 10 to 70, 10 to 80, 10 to 90, 10 to 100, 20 to 30, 20 to 40, or 20 to 50 nucleotides). In some embodiments, A and B each, independently, include at least 6, 8, 10, 12, 14, 16, 18, 20, 25, 30, 35, 40, 35, or 50 nucleotides.

In some embodiments, L is an oligonucleotide linker. In some embodiments, L includes between 4 and 100 nucleotides (e.g., 4 to 10, 4 to 20, 4 to 30, 4 to 40, 4 to 50, 4 to 60, 4 to 70, 4 to 80, 4 to 90, 4 to 100, 6 to 10, 6 to 20, 6 to 30, 6 to 40, 6 to 50, 6 to 60, 6 to 70, 6 to 80, 6 to 90, 6 to 100, 10 to 20, 10 to 30 10 to 40, 10 to 50, 10 to 60, 10 to 70, 10 to 80, 10 to 90, 10 to 100, 20 to 30, 20 to 40, or 20 to 50 nucleotides). In some embodiments, A and B each, independently, include at least 6, 8, 10, 12, 14, 16, 18, 20, 25, 30, 35, 40, 35, or 50 nucleotides.

In some embodiments, L includes a miRNA binding site. In some embodiments, L includes an endonuclease binding site.

In some embodiments, upon administration to a cell, cleavage of the linker region of the conjugate increases mRNA expression (e.g. relative to the composition wherein the linker region is not cleaved). In some embodiments, cleavage of the linker region of the conjugate increases mRNA expression to 50%, 60%, 70%, 80%, 90%, 100%, 110%, 120%, 130%, 140%, 150%, 180%, 200%, 300%, 400%, 500%, 1000% or more of the level of expression when the mRNA is administered alone.

In some embodiments, A and/or B includes at least one alternative internucleoside linkage, nucleobase analog, sugar analog, or nucleoside analog as described herein. In some embodiments, A or B includes at least one 2′-OMe nucleotide, 2′-MOE nucleotide, 2′-F nucleotide, 2′-NH₂ nucleotide, FANA nucleotide, LNA nucleotide, 4′-S nucleotide, TNA nucleotide, or PNA nucleotide. In some embodiments, A and B each include at least one 2′-OMe nucleotide, 2′-MOE nucleotide, 2′-F nucleotide, 2′-NH₂ nucleotide, FANA nucleotide, LNA nucleotide, 4′-S nucleotide, TNA nucleotide, or PNA nucleotide.

In some embodiments, A includes a region of linked nucleotides (e.g., a region of 5 or more, 10 or more, 15 or more, 20 or more, 25 or more, 30 or more, 35 or more, 40 or more, 45 or more, or 50 or more linked nucleotides) complementary to a portion of the sequence of the 5′-UTR, the 3′-UTR, the open reading frame, the start codon, the stop codon, or poly-A region of the mRNA. In some embodiments, A includes a region of linked nucleotides complementary to a portion of the sequence of the 5′-UTR.

In some embodiments, B includes a region of linked nucleotides (e.g., a region of 5 or more, 10 or more, 15 or more, 20 or more, 25 or more, 30 or more, 35 or more, 40 or more, 45 or more, or 50 or more linked nucleotides) complementary to a portion of the sequence of the 5′-UTR, the 3′-UTR, the open reading frame, the start codon, the stop codon, or poly-A region of the mRNA. In some embodiments, B includes a region of linked nucleotides complementary to a portion of the sequence of the 3′-UTR.

In some embodiments, the mRNA and conjugate are hybridized.

In some embodiments, the conjugate further includes a moiety selected from a sterol, a polyethylene glycol, a polylactic acid, a sugar, a toll-like receptor antagonist, or an endosomal escape peptide. In some embodiments, the moiety is a sterol. In some embodiments, the sterol is cholesterol. In some embodiments, the moiety is conjugated to the conjugate via a linker. In some embodiments, the moiety is conjugated to the 5′-terminus of the oligonucleotide, the 3′-terminus of the oligonucleotide, or an internal nucleotide via a linkage to the Hoogsteen face of a nucleobase.

In some embodiments, the composition further includes: (c) a second oligonucleotide including a region of linked nucleotides complimentary to at least a second portion of the sequence of the mRNA.

In another aspect, the invention features a method for selective expression of an mRNA in one or more cell types, wherein the method includes administering to a subject (e.g., a human subject) the composition including: (a) an mRNA encoding a polypeptide including: (i) a 5′-cap structure; (ii) a 5′-UTR; (iii) an open reading frame encoding the polypeptide; (iv) a 3′-UTR; and (v) a poly-A region; and (b) a conjugate including the structure: A-L-B; wherein A is a first oligonucleotide, L is a linker including a cleavage site, and B is a second oligonucleotide, wherein A and B each include a region of linked nucleotides complimentary to a different portion of the sequence of the mRNA; wherein the cleavage site is selectively cleaved in the one or more cell types.

In another aspect, the invention features a composition including: (a) a first mRNA encoding a polypeptide including: (i) a 5′-cap structure; (ii) a 5′-UTR; (iii) an open reading frame encoding the polypeptide; (iv) a 3′-UTR; and (v) a poly-A region; and (b) a second mRNA encoding a polypeptide including: (i) a 5′-cap structure; (ii) a 5′-UTR; (iii) an open reading frame encoding the polypeptide; (iv) a 3′-UTR; and (v) a poly-A region; and (c) a conjugate including the structure: A-L-B; wherein A is a first oligonucleotide including a region of linked nucleotides complimentary to a portion of the sequence of the first mRNA, L is a linker, and B is a second oligonucleotide including a region of linked nucleotides complimentary to a portion of the sequence of the second mRNA.

In some embodiments, A and B each, independently, include between 6 and 100 nucleotides (e.g., 6 to 10, 6 to 20, 6 to 30, 6 to 40, 6 to 50, 6 to 60, 6 to 70, 6 to 80, 6 to 90, 10 to 20, 10 to 30 10 to 40, 10 to 50, 10 to 60, 10 to 70, 10 to 80, 10 to 90, 10 to 100, 20 to 30, 20 to 40, or 20 to 50 nucleotides). In some embodiments, A and B each, independently, include at least 6, 8, 10, 12, 14, 16, 18, 20, 25, 30, 35, 40, 35, or 50 nucleotides.

In some embodiments, L is an oligonucleotide linker. In some embodiments, L includes between 4 and 100 nucleotides (e.g., 4 to 10, 4 to 20, 4 to 30, 4 to 40, 4 to 50, 4 to 60, 4 to 70, 4 to 80, 4 to 90, 4 to 100, 6 to 10, 6 to 20, 6 to 30, 6 to 40, 6 to 50, 6 to 60, 6 to 70, 6 to 80, 6 to 90, 6 to 100, 10 to 20, 10 to 30 10 to 40, 10 to 50, 10 to 60, 10 to 70, 10 to 80, 10 to 90, 10 to 100, 20 to 30, 20 to 40, or 20 to 50 nucleotides). In some embodiments, A and B each, independently, include at least 6, 8, 10, 12, 14, 16, 18, 20, 25, 30, 35, 40, 35, or 50 nucleotides.

In some embodiments, L includes a miRNA binding site. In some embodiments, L includes an endonuclease binding site.

In some embodiments, A and/or B includes at least one alternative internucleoside linkage, nucleobase analog, sugar analog, or nucleoside analog as described herein. In some embodiments, A or B includes at least one 2′-OMe nucleotide, 2′-MOE nucleotide, 2′-F nucleotide, 2′-NH₂ nucleotide, FANA nucleotide, LNA nucleotide, 4′-S nucleotide, TNA nucleotide, or PNA nucleotide. In some embodiments, A and B each include at least one 2′-OMe nucleotide, 2′-MOE nucleotide, 2′-F nucleotide, 2′-NH₂ nucleotide, FANA nucleotide, LNA nucleotide, 4′-S nucleotide, TNA nucleotide, or PNA nucleotide.

In some embodiments, A includes a region of linked nucleotides (e.g., a region of 5 or more, 10 or more, 15 or more, 20 or more, 25 or more, 30 or more, 35 or more, 40 or more, 45 or more, or 50 or more linked nucleotides) complementary to a portion of the sequence of the 5′-UTR, the 3′-UTR, the open reading frame, the start codon, the stop codon, or poly-A region of the mRNA.

In some embodiments, B includes a region of linked nucleotides (e.g., a region of 5 or more, 10 or more, 15 or more, 20 or more, 25 or more, 30 or more, 35 or more, 40 or more, 45 or more, or 50 or more linked nucleotides) complementary to a portion of the sequence of the 5′-UTR, the 3′-UTR, the open reading frame, the start codon, the stop codon, or poly-A region of the mRNA.

In some embodiments, the first mRNA is hybridized to A and the second mRNA is hybridized to B.

In some embodiments, the composition further includes: (d) a third mRNA encoding a polypeptide including: (i) a 5′-cap structure; (ii) a 5′-UTR; (iii) an open reading frame encoding the polypeptide; (iv) a 3′-UTR; and (v) a poly-A region; and (c) a second conjugate including the structure: C-L-D; wherein C is a first oligonucleotide including a region of linked nucleotides complimentary to a portion of the sequence of the first or the second mRNA, L is a linker, and D is a second oligonucleotide including a region of linked nucleotides complimentary to a portion of the sequence of the third mRNA.

In another aspect, the invention features a composition including: (a) an mRNA encoding a polypeptide including: (i) a 5′-cap structure; (ii) a 5′-UTR; (iii) an open reading frame encoding the polypeptide; (iv) a 3′-untranslated region (3′-UTR); and (v) a poly-A region; and (b) an oligonucleotide including a region of linked nucleotides complementary to a portion of the sequence of the mRNA including the 3′-terminus of the mRNA, wherein the oligonucleotide includes a stem-loop structure.

In some embodiments, the binding of the oligonucleotide to the mRNA produces a triple helix structure at the 3′ terminus of the mRNA. In some embodiments, the binding of the oligonucleotide to the mRNA produces a stem-loop structure at the 3′ terminus of the mRNA.

In some embodiments, the oligonucleotide comprises between 10 and 200 nucleotides (e.g., 10-20, 20-30, 30-40, 40-50, 50-60, 60-70, 70-80, 80-90, 90-100, 10-50, 50-100, 100-150, or 150-200 nucleotides).

In some embodiments, the portion of the sequence of the mRNA including the 3′ terminus includes between 6 and 100 nucleotides (e.g., 6 to 10, 6 to 20, 6 to 30, 6 to 40, 6 to 50, 6 to 60, 6 to 70, 6 to 80, 6 to 90, 10 to 20, 10 to 30 10 to 40, 10 to 50, 10 to 60, 10 to 70, 10 to 80, 10 to 90, 10 to 100, 20 to 30, 20 to 40, 20 to 50, or 50-100 nucleotides). The nucleotides may be a continuous portion of the mRNA including the 3′ terminus of the mRNA.

In another aspect, the invention features a double-stranded RNA including (a) a first strand having (i) a 5′-cap structure; (ii) a 5′-UTR; (iii) an open reading frame encoding the polypeptide; (iv) a 3′-untranslated region (3′-UTR); and (v) a poly-A region; and (b) a second strand including one or more oligonucleotides including two regions of linked nucleotides complementary to non-contiguous portions of the sequence of the mRNA.

In some embodiments, the double-stranded RNA is more compact than a corresponding RNA including only the first strand. In some embodiments, when administered to a cell, the double-stranded RNA has a longer half-life (e.g., a reduced rate of hydrolysis and/or increased resistance to nucleases) compared to a corresponding RNA including only the first strand. In some embodiments, when administered to a cell in the absence of a lipid nanoparticle the double-stranded RNA results in greater expression compared to a corresponding RNA including only the first strand. In some embodiments, when contacted with an LNP, the double-stranded RNA has greater loading compared to a corresponding RNA including only the first strand.

In some embodiments of any of the foregoing compositions, the oligonucleotide includes at least one alternative internucleoside linkage, nucleobase analog, sugar analog, or nucleoside analog as described herein. In some embodiments, each oligonucleotide includes at least one 2′-OMe nucleotide, 2′-MOE nucleotide, 2′-F nucleotide, 2′-NH₂ nucleotide, FANA nucleotide, LNA nucleotide, 4′-S nucleotide, TNA nucleotide, or PNA nucleotide. In some embodiments, each oligonucleotide includes at least one 2′-OMe nucleotide. In some embodiments, each oligonucleotide consists of 2′-OMe nucleotides.

In some embodiments, the mRNA is hybridized to the oligonucleotide.

In some embodiments of any of the aspect described herein, the oligonucleotide or conjugate further includes (e.g. is covalently conjugated to, such as, via a linker) a moiety selected from a sterol, a polyethylene glycol, a polylactic acid, a sugar, a toll-like receptor antagonist, or an endosomal escape peptide.

In some embodiments, the moiety is a sterol. In some embodiments, the sterol is selected from adosterol, agosterol A, atheronals, avenasterol, azacosterol, blazein, a blood lipid, cerevisterol, cholesterol, cholesterol sulfate, colestolone, cycloartenol, daucosterol, 7-dehydrocholesterol, 5-dehydroepisterol, 7-dehydrositosterol, 20α,22R-dihydroxycholesterol, dinosterol, epibrassicasterol, episterol, ergosterol, ergosterol, fecosterol, fucosterol, fungisterol, ganoderenic acid, ganoderic acid, ganoderiol, ganodermadiol, 7α-hydroxycholesterol, 22R-hydroxycholesterol, 27-hydroxycholesterol, inotodiol, lanosterol, lathosterol, lichesterol, lucidadiol, lumisterol, oxycholesterol, oxysterol, parkeol, saringosterol, spinasterol, sterol ester, trametenolic acid, zhankuic acid, or zymosterol. In some embodiments, the sterol is cholesterol.

In some embodiment, the oligonucleotide or conjugate includes (e.g. is covalently conjugated to, such as, via a linker) 2 or more moieties, 3 or more moieties, 4 or more moieties, or 5 or more moieties. This may include moieties of the same type, or moieties of different types. In some embodiments, the moiety is conjugated to a nucleotide by way of a linker. In some embodiments, the linker includes a PEG linker. In some embodiments, the moiety is conjugated to the 5′-terminus of the oligonucleotide, the 3′-terminus of the oligonucleotide, or an internal nucleotide via a linkage to the Hoogsteen face of a nucleobase.

In some embodiments, the oligonucleotide or conjugate includes 2 or more sterols, 3 or more sterols, 4 or more sterols, or 5 or more sterols. This may include sterols of the same type (e.g., multiple cholesterol moieties conjugates to a single oligonucleotide or conjugate), or sterols of different types. In some embodiments, the sterol is conjugated to a nucleotide by way of a linker. In some embodiments, the linker includes a PEG linker. In some embodiments, the sterol is conjugated to the 5′-terminus of the oligonucleotide, the 3′-terminus of the oligonucleotide, or an internal nucleotide via a linkage to the Hoogsteen face of a nucleobase.

In another aspect, the invention features a pharmaceutical composition including a composition of any of the aspects described herein and a pharmaceutically-acceptable excipient.

In some embodiments of any of the aspects described herein, the composition or pharmaceutical composition is administered to a cell and/or a subject (e.g., a human subject).

In some embodiments of any of the aspects described herein, the composition is associated with a lipid nanoparticle.

In some embodiments of any of the aspects described herein, upon administration to a cell (e.g., administration to a subject, such as a human subject), the composition results in an expression level of the encoded polypeptide that is at least 10%, 20%, 30%, 40%, 50%, 60%, 70%, 80%, 90%, 95%, or 99% of the expression level of the encoded polypeptide when the mRNA is administered alone.

In some embodiments of any of the aspects described herein, upon administration to a cell (e.g., administration to a subject, such as a human subject), the composition results in an expression level of the encoded polypeptide that is greater than the expression level of the encoded polypeptide when the mRNA is administered alone. In some embodiments, the composition results in an expression level of the encoded polypeptide that is at least 10%, 20%, 30%, 40%, 50%, 60%, 70%, 80%, 90%, 100%, 150%, 200%, 500%, or 1000% greater than the expression level of the encoded polypeptide when the mRNA is administered alone. In some embodiments, the composition results in an expression level of the encoded polypeptide that is at least 2 times, 3 times, 4 times, 5 times, 10 times, 20 times, 50 times, or 100 times greater than the expression level of the encoded polypeptide when the mRNA is administered alone.

In some embodiments of any of the aspects described herein, upon administration to a cell (e.g., administration to a subject, such as a human subject), the composition induces a lower innate immune response compared to the mRNA alone. In some embodiments, the composition induces an innate immune response that is at least 10%, 20%, 30%, 40%, 50%, 60%, 70%, 80%, 90%, 100%, 150%, 200%, 500%, or 1000% lower than the innate immune response induced when the mRNA is administered alone. In some embodiments, the composition induces an innate immune response that is at least 2 times, 3 times, 4 times, 5 times, 10 times, 20 times, 50 times, or 100 times lower than the innate immune response induced when the mRNA is administered alone.

In another aspect, the invention features a method of increasing gene expression in a cell (e.g., a cell of a subject, such as a human subject), the method including delivering to a cell (e.g., delivering to a subject, such as a human subject) a composition or a pharmaceutical composition, such as any of the compositions or pharmaceutical compositions described herein.

In another aspect, the invention features a method of producing a composition described herein, wherein the method includes combining the oligonucleotide or oligonucleotides (e.g., a conjugate of the invention including an oligonucleotide or oligonucleotides) and the mRNA under conditions sufficient to allow for the hybridization of the oligonucleotides to the mRNA. In some embodiments, the sufficient conditions include heating a solution including the mRNA and the oligonucleotide followed by cooling the solution. In some embodiments, the solution including the mRNA and the oligonucleotide is an aqueous solution. In some embodiments, the solution further includes an inorganic salt. In some embodiments, the solution further includes a chelating agent.

Other features and advantages of the present disclosure will be apparent from the following detailed description and figures, and from the claims.

### BRIEF DESCRIPTION OF THE DRAWINGS

FIG. 1 is a size-exclusion chromatograph showing the associated of oligonucleotides with mRNA, as described in Example 11.

FIG. 2 is a series of size-exclusion chromatographs showing the association of oligonucleotides conjugated to bulky moieties with mRNA, as described in Example 11.

FIG. 3 is a series of size-exclusion chromatographs showing the requirement for sequence complementarity for association of an oligonucleotide with mRNA, as described in Example 11.

FIG. 4 is a schematic showing the influence of length and location for association of an oligonucleotide with mRNA, as described in Example 11.

FIG. 5 is a graph showing dependence on location of the oligonucleotide binding to the mRNA on mRNA expression, as described in Example 12.

FIG. 6 is a graph showing the quantification of the innate immune response in cells in response to mRNA in complex with one or more oligonucleotides, as described in Example 13.

FIG. 7 is a series of graphs showing the quantification of the innate immune response in vivo in mice (% activated B cells in spleen) in response to mRNA in complex with one or more oligonucleotides, as described in Example 13.

FIG. 8 is a series of graphs showing the quantification of the innate immune response in vivo in mice (CD9+, CD19+CD86+, CD69+ B cell immune response) in response to mRNA in complex with one or more oligonucleotides, as described in Example 13.

FIG. 9 is a graph showing in vivo expression in mice of an mRNA in complex with one or more oligonucleotides, as described in Example 14.

FIG. 10 is a graph showing in vivo expression in mice of an mRNA in complex with one or more oligonucleotides (hEPO expression at 6 h, 6 CD-1 mice/group, IV, 0.5 mg/kg), as described in Example 14.

FIG. 11 is a graph showing in vivo expression in mice of an mRNA in complex with one or more oligonucleotides (hEPO expression at 24 h, 6 CD-1 mice/group, IV, 0.5 mg/kg), as described in Example 14.

FIG. 12 is a graph showing the serum half-life of an mRNA in complex with one or more oligonucleotides, as described in Example 15.

FIG. 13 is a graph showing the reduction of mRNA expression by complexation with one or more oligonucleotides, as described in Example 16.

FIG. 14 is a graph showing the reduction of mRNA expression by complexation with one or more oligonucleotides, where complexation induces a loop structure in the mRNA, as described in Example 16.

FIG. 15 is a graph showing oligonucleotide-induced mRNA loop geometry measured by fluorescence resonance energy transfer (FRET), as described in Example 17.

FIG. 16 is a graph showing the effect of oligonucleotide-induced compaction on mRNA expression, as described in Example 18.

FIG. 17 is a series of chromatographs showing the effect of oligonucleotide-induced compaction on mRNA integrity, as described in Example 18.

FIG. 18 is graph showing the effect of oligonucleotide-induced compaction on mRNA integrity following incubation for 6 days at 37° C., as described in Example 18.

FIG. 19 is a series of chromatographs showing the effect of oligonucleotide-induced compaction on mRNA integrity at 0 days and 5 days incubation at 37° C., as described in Example 18.

FIG. 20 is a series of chromatographs showing an oligonucleotide that binds to two separate mRNAs, as described in Example 19.

FIG. 21 is a schematic showing cholesterol-oligonucleotide conjugates, as described in Example 20.

FIG. 22 is a series of chromatographs showing the association of cholesterol-conjugated oligonucleotides with mRNA, as described in Example 20.

FIG. 23 is a graph showing the expression of mRNA bound to one or more cholesterol-oligonucleotide conjugates, as described in Example 20.

FIG. 24 is a graph showing the expression of mRNA bound to one or more cholesterol-oligonucleotide conjugates, as described in Example 20.

FIG. 25 is a graph showing a reduction in induced innate immune response following complexation of cholesterol-conjugated oligonucleotides to mRNA (10:1 conjugate:mRNA molar ratio), as described in Example 21.

FIG. 26 is a graph showing a reduction in induced innate immune response following complexation of cholesterol-conjugated oligonucleotides to mRNA (1:1 conjugate:mRNA molar ratio), as described in Example 21.

FIG. 27 is a series of size-exclusion chromatographs showing the association of an oligonucleotide with the 3′ terminus of an mRNA where binding of the oligonucleotide to the mRNA forms a triple helix at the 3′ terminus of the mRNA, as described in Example 22.

FIG. 28 is a graph showing the expression of an mRNA following binding of an oligonucleotide to the 3′ terminus of the mRNA, where binding forms a triple helix at the 3′ terminus of the mRNA, as described in Example 22.

FIG. 29 is a graph showing the expression of an mRNA following binding of an oligonucleotide to the 3′ terminus of the mRNA, where binding forms a stem-loop at the 3′ terminus of the mRNA, as described in Example 22.

### DETAILED DESCRIPTION OF THE INVENTION

The present disclosure provides methods and compositions for modulating protein expression. In particular, the invention features methods and compositions for increasing protein expression in a cell by delivering to the cell a composition including an mRNA encoding a polypeptide and one or more oligonucleotides, wherein each of the one or more oligonucleotides includes a region of linked nucleotides complimentary to a portion of the sequence of the mRNA. The methods and compositions described herein may be used to modulate gene expression (e.g., increase gene expression), to increase the stability of the mRNA, to decrease the immunogenicity of the mRNA, to enable selective expression (e.g., in a target cell or tissue) of the mRNA, and/or to enable the delivery of two or more mRNAs in a stoichiometric ratio.

### Compositions of the Invention

The present disclosure provides compositions including one or more mRNAs and one or more oligonucleotides, wherein each oligonucleotide includes a region of linked nucleotides complimentary to a portion of the sequence of the mRNA.

In one aspect, the invention features a composition including: (a) an mRNA encoding a polypeptide including: (i) a 5′-cap structure; (ii) a 5′-UTR; (iii) an open reading frame encoding the polypeptide; (iv) a 3′-UTR; and (v) a poly-A region; and (b) three or more oligonucleotides (e.g., 3, 4, 5, 6, 7, 8, 9, 10, 15, 20, 25, 30, 35, 40, 45, or 50 or more oligonucleotides), wherein each oligonucleotide includes a region of linked nucleotides complimentary to a different portion of the sequence of the mRNA.

In another aspect, the invention features a composition including: (a) an mRNA encoding a polypeptide including: (i) a 5′-cap structure; (ii) a 5′-UTR; (iii) an open reading frame encoding the polypeptide; (iv) a 3′-UTR; and (v) a poly-A region; and (b) a conjugate including an oligonucleotide including a region of linked nucleotides complimentary to a portion of the sequence of the mRNA and at least one sterol moiety (e.g., cholesterol).

In another aspect, the invention features a composition including: (a) an mRNA encoding a polypeptide including: (i) a 5′-cap structure; (ii) a 5′-UTR; (iii) an open reading frame encoding the polypeptide; (iv) a 3′-UTR; and (v) a poly-A region; and (b) a conjugate including the structure: A-L-B; wherein A is a first oligonucleotide, L is a linker including a cleavage site, and B is a second oligonucleotide, wherein A and B each include a region of linked nucleotides complimentary to a different portion of the sequence of the mRNA.

In another aspect, the invention features a composition including: (a) a first mRNA encoding a polypeptide including: (i) a 5′-cap structure; (ii) a 5′-UTR; (iii) an open reading frame encoding the polypeptide; (iv) a 3′-UTR; and (v) a poly-A region; and (b) a second mRNA encoding a polypeptide including: (i) a 5′-cap structure; (ii) a 5′-UTR; (iii) an open reading frame encoding the polypeptide; (iv) a 3′-UTR; and (v) a poly-A region; and (c) a conjugate including the structure: A-L-B; wherein A is a first oligonucleotide including a region of linked nucleotides complimentary to a portion of the sequence of the first mRNA, L is a linker, and B is a second oligonucleotide including a region of linked nucleotides complimentary to a portion of the sequence of the second mRNA.

In another aspect, the invention features a composition including: (a) an mRNA encoding a polypeptide including: (i) a 5′-cap structure; (ii) a 5′-UTR; (iii) an open reading frame encoding the polypeptide; (iv) a 3′-untranslated region (3′-UTR); and (v) a poly-A region; and (b) an oligonucleotide including a region of linked nucleotides complementary to a portion of the sequence of the mRNA, wherein the portion of the sequence of the mRNA includes the 3′ terminus of the poly-A region of the mRNA, and wherein binding of the oligonucleotide to the mRNA produces a triple helix or a stem-loop structure at the 3′ terminus of the poly-A region of the mRNA.

In another aspect, the invention features a double-stranded RNA including (a) a first strand having (i) a 5′-cap structure; (ii) a 5′-UTR; (iii) an open reading frame encoding the polypeptide; (iv) a 3′-untranslated region (3′-UTR); and (v) a poly-A region; and (b) a second strand including one or more oligonucleotides including two regions of linked nucleotides complementary to non-contiguous portions of the sequence of the mRNA.

### Nucleic Acids Conjugated to One or More Moieties

A nucleic acid (e.g., an mRNA or an oligonucleotide) of any composition of the invention may include (e.g., be covalently conjugated to, such as via a linker) one or more moieties. In preferred embodiments, one or more oligonucleotides of any composition of the invention is conjugated to one or more moieties. Wherein the composition includes more than one oligonucleotide, each oligonucleotide may be independently conjugated to one or more moieties, including one or more different moieties.

In some embodiments, each moiety is selected from a sterol, a polyethylene glycol, a polylactic acid, a sugar (e.g., GalNac), a toll-like receptor antagonist, a folate, vitamin A, biotin, an aptamer, a lipid, or a peptide (e.g., an endosomal escape peptide).

The moiety may be conjugated to a nucleic acid (e.g., an oligonucleotide) via a linker. In some embodiments, the moiety is conjugated to the 5′-terminus of the oligonucleotide, the 3′-terminus of the oligonucleotide, or an internal nucleotide via a linkage to the Hoogsteen face of a nucleobase. Exemplary methods for the conjugation of a moiety to a nucleic acid are described herein and further methods are known to those of skill in the art.

The nucleobase of the nucleotide can be covalently linked at any chemically appropriate position to a moiety. For example, the nucleobase can be deaza-adenosine or deaza-guanosine and the linker can be attached at the C-7 or C-8 positions of the deaza-adenosine or deaza-guanosine. In other embodiments, the nucleobase can be cytosine or uracil and the linker can be attached to the N-3 or C-5 positions of cytosine or uracil.

Sterols

In some embodiments, the moiety is a sterol. In some embodiments, the sterol is selected from adosterol, agosterol A, atheronals, avenasterol, azacosterol, blazein, a blood lipid, cerevisterol, cholesterol, cholesterol sulfate, colestolone, cycloartenol, daucosterol, 7-dehydrocholesterol, 5-dehydroepisterol, 7-dehydrositosterol, 20α,22R-dihydroxycholesterol, dinosterol, epibrassicasterol, episterol, ergosterol, ergosterol, fecosterol, fucosterol, fungisterol, ganoderenic acid, ganoderic acid, ganoderiol, ganodermadiol, 7α-hydroxycholesterol, 22R-hydroxycholesterol, 27-hydroxycholesterol, inotodiol, lanosterol, lathosterol, lichesterol, lucidadiol, lumisterol, oxycholesterol, oxysterol, parkeol, saringosterol, spinasterol, sterol ester, trametenolic acid, zhankuic acid, or zymosterol. In preferred embodiments, the sterol is cholesterol.

Therapeutic Agents

In some embodiments the moiety is a therapeutic agent such as a cytotoxin, radioactive ion, chemotherapeutic, or other therapeutic agent. A cytotoxin or cytotoxic agent includes any agent that is detrimental to cells. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, puromycin, maytansinoids, e.g., maytansinol (see U.S. Pat. No. 5,208,020), CC-1065 (see U.S. Pat. Nos. 5,475,092, 5,585,499, 5,846,545) and analogs or homologs thereof. Radioactive ions include, but are not limited to iodine (e.g., iodine 125 or iodine 131), strontium 89, phosphorous, palladium, cesium, iridium, phosphate, cobalt, yttrium 90, Samarium 153 and praseodymium. Other therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, CC-1065, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine, vinblastine, taxol and maytansinoids).

Detectable Agents

In some embodiments, the moiety is a detectable agent. Examples of detectable substances include various organic small molecules, inorganic compounds, nanoparticles, enzymes or enzyme substrates, fluorescent materials, luminescent materials, bioluminescent materials, chemiluminescent materials, radioactive materials, and contrast agents. Such optically-detectable labels include for example, without limitation, 4-acetamido-4′-isothiocyanatostilbene-2,2′disulfonic acid; acridine and derivatives: acridine, acridine isothiocyanate; 5-(2′-aminoethyl)aminonaphthalene-1-sulfonic acid (EDANS); 4-amino-N-[3-vinylsulfonyl)phenyl]naphthalimide-3,5 disulfonate; N-(4-anilino-1-naphthyl)maleimide; anthranilamide; BODIPY; Brilliant Yellow; coumarin and derivatives; coumarin, 7-amino-4-methylcoumarin (AMC, Coumarin 120), 7-amino-4-trifluoromethylcouluarin (Coumaran 151); cyanine dyes; cyanosine; 4′,6-diaminidino-2-phenylindole (DAPI); 5′5″-dibromopyrogallol-sulfonaphthalein (Bromopyrogallol Red); 7-diethylamino-3-(4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2′-disulfonic acid; 5-[dimethylamino]-naphthalene-1-sulfonyl chloride (DNS, dansylchloride); 4-dimethylaminophenylazophenyl-4′-isothiocyanate (DABITC); eosin and derivatives; eosin, eosin isothiocyanate, erythrosin and derivatives; erythrosin B, erythrosin, isothiocyanate; ethidium; fluorescein and derivatives; 5-carboxyfluorescein (FAM), 5-(4,6-dichlorotriazin-2-yl)aminofluorescein (DTAF), 2′,7′-dimethoxy-4′5′-dichloro-6-carboxyfluorescein, fluorescein, fluorescein isothiocyanate, QFITC, (XRITC); fluorescamine; IR144; IR1446; Malachite Green isothiocyanate; 4-methylumbelliferoneortho cresolphthalein; nitrotyrosine; pararosaniline; Phenol Red; B-phycoerythrin; o-phthaldialdehyde; pyrene and derivatives: pyrene, pyrene butyrate, succinimidyl 1-pyrene; butyrate quantum dots; Reactive Red 4 (Cibacron™ Brilliant Red 3B-A) rhodamine and derivatives: 6-carboxy-X-rhodamine (ROX), 6-carboxyrhodamine (R6G), lissamine rhodamine B sulfonyl chloride rhodarnine (Rhod), rhodamine B, rhodamine 123, rhodamine X isothiocyanate, sulforhodamine B, sulforhodamine 101, sulfonyl chloride derivative of sulforhodamine 101 (Texas Red); N,N,N′,N′tetramethyl-6-carboxyrhodamine (TAMRA); tetramethyl rhodamine; tetramethyl rhodamine isothiocyanate (TRITC); riboflavin; rosolic acid; terbium chelate derivatives; Cyanine-3 (Cy3); Cyanine-5 (Cy5); Cyanine-5.5 (Cy5.5), Cyanine-7 (Cy7); IRD 700; IRD 800; Alexa 647; La Jolta Blue; phthalo cyanine; and naphthalo cyanine. In some embodiments, the detectable label is a fluorescent dye, such as Cy5 and Cy3.

An example of a luminescent material is luminol; bioluminescent materials include luciferase, luciferin, and aequorin.

Suitable radioactive material include ¹⁸F, ⁶⁷Ga, ^{81m}Kr, ⁸²Rb, ¹¹¹In, ¹²³I, ¹³³Xe, ²⁰¹Tl, ¹²⁵I, ³⁵S, ¹⁴C, or ³H, ^{99m}Tc (e.g., as pertechnetate (technetate(VII), TcO₄⁻) either directly or indirectly, or other radioisotope detectable by direct counting of radioemission or by scintillation counting.

In addition, contrast agents, e.g., contrast agents for MRI or NMR, for X-ray CT, Raman imaging, optical coherence tomography, absorption imaging, ultrasound imaging, or thermal imaging can be used. Exemplary contrast agents include gold (e.g., gold nanoparticles), gadolinium (e.g., chelated Gd), iron oxides (e.g., superparamagnetic iron oxide (SPIO), monocrystalline iron oxide nanoparticles (MIONs), and ultrasmall superparamagnetic iron oxide (USPIO)), manganese chelates (e.g., Mn-DPDP), barium sulfate, iodinated contrast media (iohexol), microbubbles, or perfluorocarbons can also be used.

In some embodiments, the detectable agent is a non-detectable pre-cursor that becomes detectable upon activation. Examples include fluorogenic tetrazine-fluorophore constructs (e.g., tetrazine-BODIPY FL, tetrazine-Oregon Green 488, or tetrazine-BODIPY TMR-X) or enzyme activatable fluorogenic agents (e.g., PROSENSE (VisEn Medical)).

When the compounds are enzymatically labeled with, for example, horseradish peroxidase, alkaline phosphatase, or luciferase, the enzymatic label is detected by determination of conversion of an appropriate substrate to product.

In vitro assays in which these compositions can be used include enzyme linked immunosorbent assays (ELISAs), immunoprecipitations, immunofluorescence, enzyme immunoassay (EIA), radioimmunoassay (RIA), and Western blot analysis.

Labels can be attached to the nucleotide of the present disclosure at any position using standard chemistries such that the label can be removed from the incorporated base upon cleavage of the cleavable linker.

Cell Penetrating Peptides

In some embodiments, the moiety is a cell penetrating moiety or agent that enhances intracellular delivery of the compositions. For example, the compositions can include a cell-penetrating peptide sequence that facilitates delivery to the intracellular space, e.g., HIV-derived TAT peptide, penetratins, transportans, or hCT derived cell-penetrating peptides, see, e.g., Caron et al., (2001) Mol Ther. 3(3):310-8; Langel, Cell-Penetrating Peptides: Processes and Applications (CRC Press, Boca Raton Fla. 2002); El-Andaloussi et al., (2005) Curr Pharm Des. 11(28):3597-611; Deshayes et al., (2005) Cell Mol Life Sci. 62(16):1839-49; Schmitt et al., (2017) RNA. 23(9):1344-51; and Li et al., (2017) JACS. 137(44):14084-93. The compositions can also be formulated to include a cell penetrating agent, e.g., liposomes, which enhance delivery of the compositions to the intracellular space.

Biological Targets

In some embodiments, the moiety is a ligand for a biological target. The ligand can bind to the biological target either covalently or non-covalently.

Biological targets include biopolymers, e.g., antibodies, nucleic acids such as RNA and DNA, proteins, enzymes; exemplary proteins include enzymes, receptors, and ion channels. In some embodiments the target is a tissue- or cell-type specific marker, e.g., a protein that is expressed specifically on a selected tissue or cell type. In some embodiments, the target is a receptor, such as, but not limited to, plasma membrane receptors and nuclear receptors; more specific examples include G-protein-coupled receptors, cell pore proteins, transporter proteins, surface-expressed antibodies, HLA proteins, MHC proteins, and growth factor receptors.

### Linkers

A linker refers to a linkage or connection between two or more components in a compound described herein (e.g., between a nucleic acid and a moiety, such as a sterol). In some embodiments, a linker provides space, rigidity, and/or flexibility between two components in a nucleic acid or conjugate described herein. In some embodiments, a linker may be a bond, e.g., a covalent bond, e.g., an amide bond, a disulfide bond, a C—O bond, a C—N bond, a N—N bond, a C—S bond, or any kind of bond created from a chemical reaction, e.g., chemical conjugation.

In some embodiments, a linker includes no more than 250 atoms (e.g., 1-2, 1-4, 1-6, 1-8, 1-10, 1-12, 1-14, 1-16, 1-18, 1-20, 1-25, 1-30, 1-35, 1-40, 1-45, 1-50, 1-55, 1-60, 1-65, 1-70, 1-75, 1-80, 1-85, 1-90, 1-95, 1-100, 1-110, 1-120, 1-130, 1-140, 1-150, 1-160, 1-170, 1-180, 1-190, 1-200, 1-210, 1-220, 1-230, 1-240, or 1-250 atom(s); 250, 240, 230, 220, 210, 200, 190, 180, 170, 160, 150, 140, 130, 120, 110, 100, 95, 90, 85, 80, 75, 70, 65, 60, 55, 50, 45, 40, 35, 30, 28, 26, 24, 22, 20, 18, 16, 14, 12, 10, 9, 8, 7, 6, 5, 4, 3, 2, or 1 atom(s)). In some embodiments, a linker includes no more than 250 non-hydrogen atoms (e.g., 1-2, 1-4, 1-6, 1-8, 1-10, 1-12, 1-14, 1-16, 1-18, 1-20, 1-25, 1-30, 1-35, 1-40, 1-45, 1-50, 1-55, 1-60, 1-65, 1-70, 1-75, 1-80, 1-85, 1-90, 1-95, 1-100, 1-110, 1-120, 1-130, 1-140, 1-150, 1-160, 1-170, 1-180, 1-190, 1-200, 1-210, 1-220, 1-230, 1-240, or 1-250 non-hydrogen atom(s); 250, 240, 230, 220, 210, 200, 190, 180, 170, 160, 150, 140, 130, 120, 110, 100, 95, 90, 85, 80, 75, 70, 65, 60, 55, 50, 45, 40, 35, 30, 28, 26, 24, 22, 20, 18, 16, 14, 12, 10, 9, 8, 7, 6, 5, 4, 3, 2, or 1 non-hydrogen atom(s)). In some embodiments, the backbone of a linker includes no more than 250 atoms (e.g., 1-2, 1-4, 1-6, 1-8, 1-10, 1-12, 1-14, 1-16, 1-18, 1-20, 1-25, 1-30, 1-35, 1-40, 1-45, 1-50, 1-55, 1-60, 1-65, 1-70, 1-75, 1-80, 1-85, 1-90, 1-95, 1-100, 1-110, 1-120, 1-130, 1-140, 1-150, 1-160, 1-170, 1-180, 1-190, 1-200, 1-210, 1-220, 1-230, 1-240, or 1-250 atom(s); 250, 240, 230, 220, 210, 200, 190, 180, 170, 160, 150, 140, 130, 120, 110, 100, 95, 90, 85, 80, 75, 70, 65, 60, 55, 50, 45, 40, 35, 30, 28, 26, 24, 22, 20, 18, 16, 14, 12, 10, 9, 8, 7, 6, 5, 4, 3, 2, or 1 atom(s)).

A linker can be attached to a nucleic acid on one end (e.g., at the 5′ end, the 3′ end, to a nucleobase, or to a sugar of the nucleic acid) and to a moiety (e.g., any moiety described herein, such as a sterol).

A linker can include, but is not limited to the following atoms or groups: carbon, amino, alkylamino, oxygen, sulfur, sulfoxide, sulfonyl, carbonyl, and imine.

Examples of chemical groups that can be incorporated into the linker include, but are not limited to, an alkyl, alkene, an alkyne, an amido, an ether, a thioether, an or an ester group. The linker chain can also include part of a saturated, unsaturated or aromatic ring, including polycyclic and heteroaromatic rings wherein the heteroaromatic ring is an aryl group containing from one to four heteroatoms, N, O or S. Specific examples of linkers include, but are not limited to, unsaturated alkanes, polyethylene glycols, and dextran polymers.

For example, the linker can include ethylene or propylene glycol monomeric units, e.g., diethylene glycol, dipropylene glycol, triethylene glycol, tripropylene glycol, tetraethylene glycol, or tetraethylene glycol. In some embodiments, the linker can include a divalent alkyl, alkenyl, and/or alkynyl moiety. The linker can include an ester, amide, or ether moiety.

In some embodiments, a linker is a polynucleotide (e.g., a polynucleotide including 1-5, 1-10, 5-10, 10-20, 10-30, 10-40, or 10-50 nucleotides).

Other examples include cleavable moieties within the linker, such as, for example, a disulfide bond (—S—S—) or an azo bond (—N═N—), which can be cleaved using a reducing agent or photolysis. Where the linker is an oligonucleotide, the linker may include a cleavable sequence (e.g., a nucleotide sequence having an miRNA biding site or a nuclease-binding site).

Covalent conjugation of two or more components in a compound using a linker may be accomplished using well-known organic chemical synthesis techniques and methods. Complementary functional groups on two components may react with each other to form a covalent bond. Examples of complementary reactive functional groups include, but are not limited to, e.g., maleimide and cysteine, amine and activated carboxylic acid, thiol and maleimide, activated sulfonic acid and amine, isocyanate and amine, azide and alkyne, and alkene and tetrazine.

Other examples of functional groups capable of reacting with amino groups include, e.g., alkylating and acylating agents. Representative alkylating agents include: (i) an α-haloacetyl group, e.g., XCH2CO— (where X═Br, Cl, or I); (ii) a N-maleimide group, which may react with amino groups either through a Michael type reaction or through acylation by addition to the ring carbonyl group; (iii) an aryl halide, e.g., a nitrohaloaromatic group; (iv) an alkyl halide; (v) an aldehyde or ketone capable of Schiff's base formation with amino groups; (vi) an epoxide, e.g., an epichlorohydrin and a bisoxirane, which may react with amino, sulfhydryl, or phenolic hydroxyl groups; (vii) a chlorine-containing of s-triazine, which is reactive towards nucleophiles such as amino, sufhydryl, and hydroxyl groups; (viii) an aziridine, which is reactive towards nucleophiles such as amino groups by ring opening; (ix) a squaric acid diethyl ester; and (x) an α-haloalkyl ether.

Amino-reactive acylating groups include, e.g., (i) an isocyanate and an isothiocyanate; (ii) a sulfonyl chloride; (iii) an acid halide; (iv) an active ester, e.g., a nitrophenylester or N-hydroxysuccinimidyl ester; (v) an acid anhydride, e.g., a mixed, symmetrical, or N-carboxyanhydride; (vi) an acylazide; and (vii) an imidoester. Aldehydes and ketones may be reacted with amines to form Schiff's bases, which may be stabilized through reductive amination.

It will be appreciated that certain functional groups may be converted to other functional groups prior to reaction, for example, to confer additional reactivity or selectivity. Examples of methods useful for this purpose include conversion of amines to carboxyls using reagents such as dicarboxylic anhydrides; conversion of amines to thiols using reagents such as N-acetylhomocysteine thiolactone, S-acetylmercaptosuccinic anhydride, 2-iminothiolane, or thiol-containing succinimidyl derivatives; conversion of thiols to carboxyls using reagents such as α-haloacetates; conversion of thiols to amines using reagents such as ethylenimine or 2-bromoethylamine; conversion of carboxyls to amines using reagents such as carbodiimides followed by diamines; and conversion of alcohols to thiols using reagents such as tosyl chloride followed by transesterification with thioacetate and hydrolysis to the thiol with sodium acetate.

### Reduction of Immunogenicity

Innate immune response includes a cellular response to exogenous single stranded nucleic acids, generally of viral or bacterial origin, which involves the induction of cytokine expression and release, particularly the interferons, and cell death. Protein synthesis is also reduced during the innate cellular immune response. It is therefore advantageous to reduce the innate immune response in a cell which is triggered by introduction of exogenous nucleic acids. The present disclosure provides composition that substantially reduce the immune response. In some embodiments, the immune response is reduced by 10%, 20%, 30%, 40%, 50%, 60%, 70%, 80%, 90%, 95%, 99%, 99.9%, or greater than 99.9% as compared to the immune response induced by a corresponding mRNA. Such a reduction can be measured by expression or activity level of Type 1 interferons or the expression of interferon-regulated genes such as the toll-like receptors (e.g., TLR7 and TLR8). Reduction or lack of induction of innate immune response can also be measured by decreased cell death following one or more administrations of RNAs to a cell population; e.g., cell death is 10%, 25%, 50%, 75%, 85%, 90%, 95%, or over 95% less than the cell death frequency observed with a corresponding unaltered nucleic acid. Moreover, cell death may affect fewer than 50%, 40%, 30%, 20%, 10%, 5%, 1%, 0.1%, 0.01% or fewer than 0.01% of cells contacted with the alternative nucleic acids.

Compaction of mRNA by Binding to One or More Oligonucleotides

In certain embodiments of the invention, binding of one or more oligonucleotides (e.g., oligonucleotide conjugates) to an mRNA induces a geometry in the mRNA such that the mRNA bound to the one or more oligomers is more compact than the mRNA alone. The oligonucleotide may bind to two or more distinct and non-contiguous regions of the mRNA, thus inducing secondary structure in the mRNA that results in mRNA compaction.

mRNA compaction includes a reduction in the size, volume, or length of the mRNA. mRNA compaction can be determined by standard techniques known to those of skill in the art. For example, mRNA compaction can be determined by maximum ladder distance (MLD). MLD is the longest chain of edges that can be drawn within a diagram depicting the predicted most energetically stable secondary structure of a nucleic acid. MLD can be determined according to methods known to those of skill in the art, for example, as described in Borodavka et al. Sizes of long RNA molecules are determined by the branching patterns of their secondary structures. Biophysical Journal 111(10):2077-2085, 2016, which is hereby incorporated by reference in its entirety.

In some embodiments, binding of one or more oligonucleotides (e.g., oligonucleotide conjugates) to an mRNA induces a geometry in the mRNA such that the mRNA bound to the one or more oligomers is at least 10%, 20%, 30%, 40%, 50%, 60%, 70%, 80%, 90%, 100%, 200%, 400%, 500%, or 100% or more compact than the mRNA alone. In some embodiments, binding of one or more oligonucleotides (e.g., oligonucleotide conjugates) to an mRNA induces a geometry in the mRNA decreases the MLD of the mRNA bound to the one or more oligomers by at least 10%, 20%, 30%, 40%, 50%, 60%, 70%, 80%, 90%, 100%, 200%, 400%, 500%, or 100% or more than the mRNA alone.

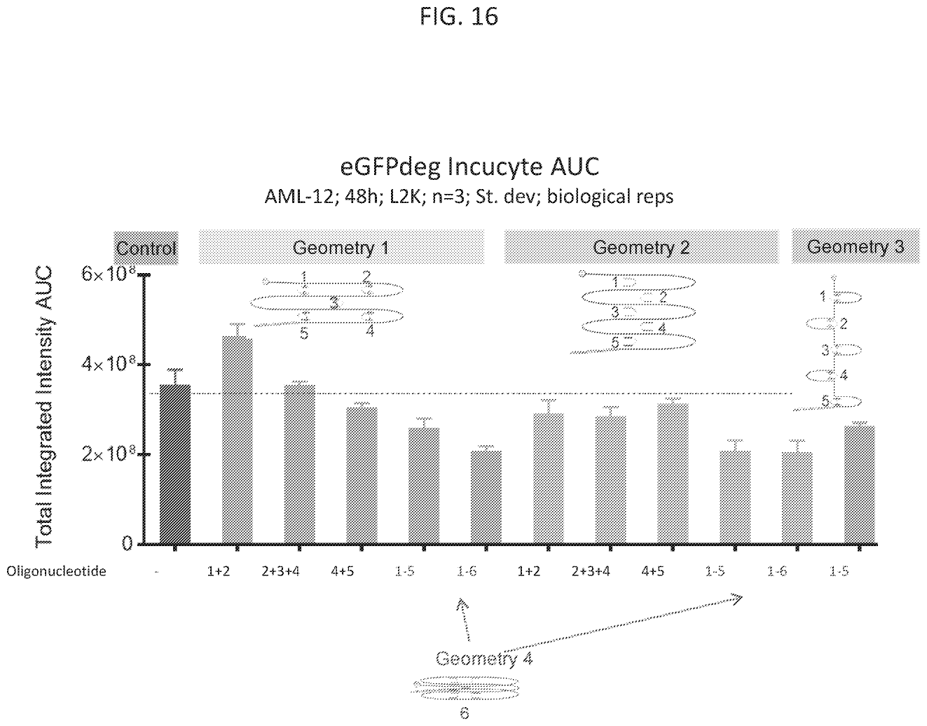

In some embodiments, the oligonucleotide conjugate has the structure of A-L-B, where A is a first oligonucleotide, L is a linker (e.g., an oligonucleotide linker), and B is a second oligonucleotide, where A and B each include a region of linked nucleotides complimentary to a different portion of the sequence of an mRNA. In some embodiments, multiple conjugates having the structure of A-L-B may hybridize with the mRNA to increase compaction of the mRNA. Exemplary mRNA secondary structures that may be induced by binding of multiple oligonucleotides having the structure of A-L-B to an mRNA are shown in FIG. 16.

mRNA compaction may increase the serum half-life of the mRNA, for example, by decreasing the rate of nuclease degradation (e.g., endonuclease and/or exonuclease degradation) and/or by decreasing the rate of hydrolysis. In some embodiments, induction of mRNA compaction increases the serum half-life of the mRNA by 5%, 10%, 20%, 30%, 40%, 50%, 60%, 70%, 80%, 90%, 100% 200% 500% or more.

mRNA compaction may increase protein expression of an mRNA, for example, by increasing the stability of the mRNA (e.g., increasing the serum half-life of the mRNA). In some embodiments, induction of mRNA compaction increases protein expression by 5%, 10%, 20%, 30%, 40%, 50%, 60%, 70%, 80%, 90%, 100% 200% 500% or more.

### MicroRNA Binding Sites

In some embodiments, nucleic acids of the invention include a sensor sequence. Sensor sequences include, for example, microRNA binding sites, transcription factor binding sites, structured mRNA sequences and/or motifs, artificial binding sites engineered to act as pseudo-receptors for endogenous nucleic acid binding molecules.

MicroRNAs (or miRNAs) are 19-25 nucleotide long noncoding RNAs that bind to the 3′-UTR of nucleic acid molecules and down-regulate gene expression either by reducing nucleic acid molecule stability or by inhibiting translation. In some embodiments, a nucleic acid of the invention, such as an oligonucleotide of the invention, comprises a miRNA binding site. Such sequences may correspond to any known microRNA such as those taught in U.S. Patent Publication Nos. 2005/0261218 and 2005/0059005, the contents of which are incorporated herein by reference in their entirety. In some embodiments, the miRNA binding site is selectively cleaved (e.g., in a particular cell or tissue type).

A microRNA sequence includes a “seed” region, i.e., a sequence in the region of positions 2-8 of the mature microRNA, which sequence has perfect Watson-Crick complementarity to the miRNA target sequence. A microRNA seed may include positions 2-8 or 2-7 of the mature microRNA. In some embodiments, a microRNA seed may include 7 nucleotides (e.g., nucleotides 2-8 of the mature microRNA), wherein the seed-complementary site in the corresponding miRNA target is flanked by an adenosine (A) opposed to microRNA position 1. In some embodiments, a microRNA seed may include 6 nucleotides (e.g., nucleotides 2-7 of the mature microRNA), wherein the seed-complementary site in the corresponding miRNA target is flanked by an adenosine (A) opposed to microRNA position 1. See for example, Grimson A, Farh K K, Johnston W K, Garrett-Engele P, Lim L P, Bartel D P; Mol Cell. 2007 Jul. 6; 27(1):91-105. The bases of the microRNA seed have complete complementarity with the target sequence. Identification of microRNA, microRNA target regions, and their expression patterns and role in biology have been reported (Bonauer et al., Curr Drug Targets 2010 11:943-949; Anand and Cheresh Curr Opin Hematol 2011 18:171-176; Contreras and Rao Leukemia 2012 26:404-413 (2011 Dec. 20. doi: 10.1038/leu.2011.356); Bartel Cell 2009 136:215-233; Landgraf et al, Cell, 2007 129:1401-1414; Gentner and Naldini, Tissue Antigens. 2012 80:393-403 and all references therein; each of which is incorporated herein by reference in its entirety).

A miRNA binding site refers to a microRNA target site or a microRNA recognition site, or any nucleotide sequence to which a microRNA binds or associates. It should be understood that “binding” may follow traditional Watson-Crick hybridization rules or may reflect any stable association of the microRNA with the target sequence at or adjacent to the microRNA site.

Examples of tissues where microRNA are known to regulate mRNA, and thereby protein expression, include, but are not limited to, liver (miR-122), muscle (miR-133, miR-206, miR-208), endothelial cells (miR-17-92, miR-126), myeloid cells (miR-142-3p, miR-142-5p, miR-16, miR-21, miR-223, miR-24, miR-27), adipose tissue (let-7, miR-30c), heart (miR-1d, miR-149), kidney (miR-192, miR-194, miR-204), and lung epithelial cells (let-7, miR-133, miR-126).

Immune cells specific microRNAs include, but are not limited to, hsa-let-7a-2-3p, hsa-let-7a-3p, hsa-7a-5p, hsa-let-7c, hsa-let-7e-3p, hsa-let-7e-5p, hsa-let-7g-3p, hsa-let-7g-5p, hsa-let-7i-3p, hsa-let-7i-5p, miR-10a-3p, miR-10a-5p, miR-1184, hsa-let-7f-1-3p, hsa-let-7f-2-5p, hsa-let-7f-5p, miR-125b-1-3p, miR-125b-2-3p, miR-125b-5p, miR-1279, miR-130a-3p, miR-130a-5p, miR-132-3p, miR-132-5p, miR-142-3p, miR-142-5p, miR-143-3p, miR-143-5p, miR-146a-3p, miR-146a-5p, miR-146b-3p, miR-146b-5p, miR-147a, miR-147b, miR-148a-5p, miR-148a-3p, miR-150-3p, miR-150-5p, miR-151b, miR-155-3p, miR-155-5p, miR-15a-3p, miR-15a-5p, miR-15b-5p, miR-15b-3p, miR-16-1-3p, miR-16-2-3p, miR-16-5p, miR-17-5p, miR-181a-3p, miR-181a-5p, miR-181a-2-3p, miR-182-3p, miR-182-5p, miR-197-3p, miR-197-5p, miR-21-5p, miR-21-3p, miR-214-3p, miR-214-5p, miR-223-3p, miR-223-5p, miR-221-3p, miR-221-5p, miR-23b-3p, miR-23b-5p, miR-24-1-5p, miR-24-2-5p, miR-24-3p, miR-26a-1-3p, miR-26a-2-3p, miR-26a-5p, miR-26b-3p, miR-26b-5p, miR-27a-3p, miR-27a-5p, miR-27b-3p, miR-27b-5p, miR-28-3p, miR-28-5p, miR-2909, miR-29a-3p, miR-29a-5p, miR-29b-1-5p, miR-29b-2-5p, miR-29c-3p, miR-29c-5p, miR-30e-3p, miR-30e-5p, miR-331-5p, miR-339-3p, miR-339-5p, miR-345-3p, miR-345-5p, miR-346, miR-34a-3p, miR-34a-5p, miR-363-3p, miR-363-5p, miR-372, miR-377-3p, miR-377-5p, miR-493-3p, miR-493-5p, miR-542, miR-548b-5p, miR548c-5p, miR-548i, miR-548j, miR-548n, miR-574-3p, miR-598, miR-718, miR-935, miR-99a-3p, miR-99a-5p, miR-99b-3p and miR-99b-5p. Furthermore, novel microRNAs are discovered in the immune cells in the art through micro-array hybridization and microtome analysis (Jima D D et al, Blood, 2010, 116:e118-e127; Vaz C et al., BMC Genomics, 2010, 11, 288, the content of each of which is incorporated herein by reference in its entirety.)

MicroRNAs that are known to be expressed in the liver include, but are not limited to, miR-107, miR-122-3p, miR-122-5p, miR-1228-3p, miR-1228-5p, miR-1249, miR-129-5p, miR-1303, miR-151a-3p, miR-151a-5p, miR-152, miR-194-3p, miR-194-5p, miR-199a-3p, miR-199a-5p, miR-199b-3p, miR-199b-5p, miR-296-5p, miR-557, miR-581, miR-939-3p, miR-939-5p.

MicroRNAs that are known to be expressed in the lung include, but are not limited to, let-7a-2-3p, let-7a-3p, let-7a-5p, miR-126-3p, miR-126-5p, miR-127-3p, miR-127-5p, miR-130a-3p, miR-130a-5p, miR-130b-3p, miR-130b-5p, miR-133a, miR-133b, miR-134, miR-18a-3p, miR-18a-5p, miR-18b-3p, miR-18b-5p, miR-24-1-5p, miR-24-2-5p, miR-24-3p, miR-296-3p, miR-296-5p, miR-32-3p, miR-337-3p, miR-337-5p, miR-381-3p, miR-381-5p.

MicroRNAs that are known to be expressed in the heart include, but are not limited to, miR-1, miR-133a, miR-133b, miR-149-3p, miR-149-5p, miR-186-3p, miR-186-5p, miR-208a, miR-208b, miR-210, miR-296-3p, miR-320, miR-451a, miR-451b, miR-499a-3p, miR-499a-5p, miR-499b-3p, miR-499b-5p, miR-744-3p, miR-744-5p, miR-92b-3p and miR-92b-5p.

MicroRNAs that are known to be expressed in the nervous system include, but are not limited to, miR-124-5p, miR-125a-3p, miR-125a-5p, miR-125b-1-3p, miR-125b-2-3p, miR-125b-5p, miR-1271-3p, miR-1271-5p, miR-128, miR-132-5p, miR-135a-3p, miR-135a-5p, miR-135b-3p, miR-135b-5p, miR-137, miR-139-5p, miR-139-3p, miR-149-3p, miR-149-5p, miR-153, miR-181c-3p, miR-181c-5p, miR-183-3p, miR-183-5p, miR-190a, miR-190b, miR-212-3p, miR-212-5p, miR-219-1-3p, miR-219-2-3p, miR-23a-3p, miR-23a-5p, miR-30a-5p, miR-30b-3p, miR-30b-5p, miR-30c-1-3p, miR-30c-2-3p, miR-30c-5p, miR-30d-3p, miR-30d-5p, miR-329, miR-342-3p, miR-3665, miR-3666, miR-380-3p, miR-380-5p, miR-383, miR-410, miR-425-3p, miR-425-5p, miR-454-3p, miR-454-5p, miR-483, miR-510, miR-516a-3p, miR-548b-5p, miR-548c-5p, miR-571, miR-7-1-3p, miR-7-2-3p, miR-7-5p, miR-802, miR-922, miR-9-3p and miR-9-5p. MicroRNAs enriched in the nervous system further include those specifically expressed in neurons, including, but not limited to, miR-132-3p, miR-132-3p, miR-148b-3p, miR-148b-5p, miR-151a-3p, miR-151a-5p, miR-212-3p, miR-212-5p, miR-320b, miR-320e, miR-323a-3p, miR-323a-5p, miR-324-5p, miR-325, miR-326, miR-328, miR-922 and those specifically expressed in glial cells, including, but not limited to, miR-1250, miR-219-1-3p, miR-219-2-3p, miR-219-5p, miR-23a-3p, miR-23a-5p, miR-3065-3p, miR-3065-5p, miR-30e-3p, miR-30e-5p, miR-32-5p, miR-338-5p, miR-657.

MicroRNAs that are known to be expressed in the pancreas include, but are not limited to, miR-105-3p, miR-105-5p, miR-184, miR-195-3p, miR-195-5p, miR-196a-3p, miR-196a-5p, miR-214-3p, miR-214-5p, miR-216a-3p, miR-216a-5p, miR-30a-3p, miR-33a-3p, miR-33a-5p, miR-375, miR-7-1-3p, miR-7-2-3p, miR-493-3p, miR-493-5p and miR-944.

MicroRNAs that are known to be expressed in the kidney further include, but are not limited to, miR-122-3p, miR-145-5p, miR-17-5p, miR-192-3p, miR-192-5p, miR-194-3p, miR-194-5p, miR-20a-3p, miR-20a-5p, miR-204-3p, miR-204-5p, miR-210, miR-216a-3p, miR-216a-5p, miR-296-3p, miR-30a-3p, miR-30a-5p, miR-30b-3p, miR-30b-5p, miR-30c-1-3p, miR-30c-2-3p, miR30c-5p, miR-324-3p, miR-335-3p, miR-335-5p, miR-363-3p, miR-363-5p and miR-562.

MicroRNAs that are known to be expressed in the muscle further include, but are not limited to, let-7g-3p, let-7g-5p, miR-1, miR-1286, miR-133a, miR-133b, miR-140-3p, miR-143-3p, miR-143-5p, miR-145-3p, miR-145-5p, miR-188-3p, miR-188-5p, miR-206, miR-208a, miR-208b, miR-25-3p and miR-25-5p.

MicroRNAs are differentially expressed in different types of cells, such as endothelial cells, epithelial cells and adipocytes. For example, microRNAs that are expressed in endothelial cells include, but are not limited to, let-7b-3p, let-7b-5p, miR-100-3p, miR-100-5p, miR-101-3p, miR-101-5p, miR-126-3p, miR-126-5p, miR-1236-3p, miR-1236-5p, miR-130a-3p, miR-130a-5p, miR-17-5p, miR-17-3p, miR-18a-3p, miR-18a-5p, miR-19a-3p, miR-19a-5p, miR-19b-1-5p, miR-19b-2-5p, miR-19b-3p, miR-20a-3p, miR-20a-5p, miR-217, miR-210, miR-21-3p, miR-21-5p, miR-221-3p, miR-221-5p, miR-222-3p, miR-222-5p, miR-23a-3p, miR-23a-5p, miR-296-5p, miR-361-3p, miR-361-5p, miR-421, miR-424-3p, miR-424-5p, miR-513a-5p, miR-92a-1-5p, miR-92a-2-5p, miR-92a-3p, miR-92b-3p and miR-92b-5p. Many novel microRNAs are discovered in endothelial cells from deep-sequencing analysis (Voellenkle C et al., RNA, 2012, 18, 472-484, herein incorporated by reference in its entirety).

For further example, microRNAs that are expressed in epithelial cells include, but are not limited to, let-7b-3p, let-7b-5p, miR-1246, miR-200a-3p, miR-200a-5p, miR-200b-3p, miR-200b-5p, miR-200c-3p, miR-200c-5p, miR-338-3p, miR-429, miR-451a, miR-451b, miR-494, miR-802 and miR-34a, miR-34b-5p, miR-34c-5p, miR-449a, miR-449b-3p, miR-449b-5p specific in respiratory ciliated epithelial cells; let-7 family, miR-133a, miR-133b, miR-126 specific in lung epithelial cells; miR-382-3p, miR-382-5p specific in renal epithelial cells and miR-762 specific in corneal epithelial cells.

In addition, a large group of microRNAs are enriched in embryonic stem cells, controlling stem cell self-renewal as well as the development and/or differentiation of various cell lineages, such as neural cells, cardiac, hematopoietic cells, skin cells, osteogenic cells and muscle cells (Kuppusamy K T et al., Curr. Mol Med, 2013, 13(5), 757-764; Vidigal J A and Ventura A, Semin Cancer Biol. 2012, 22(5-6), 428-436; Goff L A et al., PLoS One, 2009, 4:e7192; Morin R D et al., Genome Res, 2008, 18, 610-621; Yoo J K et al., Stem Cells Dev. 2012, 21(11), 2049-2057, each of which is herein incorporated by reference in its entirety). MicroRNAs abundant in embryonic stem cells include, but are not limited to, let-7a-2-3p, let-a-3p, let-7a-5p, let7d-3p, let-7d-5p, miR-103a-2-3p, miR-103a-5p, miR-106b-3p, miR-106b-5p, miR-1246, miR-1275, miR-138-1-3p, miR-138-2-3p, miR-138-5p, miR-154-3p, miR-154-5p, miR-200c-3p, miR-200c-5p, miR-290, miR-301a-3p, miR-301a-5p, miR-302a-3p, miR-302a-5p, miR-302b-3p, miR-302b-5p, miR-302c-3p, miR-302c-5p, miR-302d-3p, miR-302d-5p, miR-302e, miR-367-3p, miR-367-5p, miR-369-3p, miR-369-5p, miR-370, miR-371, miR-373, miR-380-5p, miR-423-3p, miR-423-5p, miR-486-5p, miR-520c-3p, miR-548e, miR-548f, miR-548g-3p, miR-548g-5p, miR-548i, miR-548k, miR-548l, miR-548m, miR-548n, miR-5480-3p, miR-5480-5p, miR-548p, miR-664a-3p, miR-664a-5p, miR-664b-3p, miR-664b-5p, miR-766-3p, miR-766-5p, miR-885-3p, miR-885-5p, miR-93-3p, miR-93-5p, miR-941, miR-96-3p, miR-96-5p, miR-99b-3p and miR-99b-5p. Many predicted novel microRNAs are discovered by deep sequencing in human embryonic stem cells (Morin R D et al., Genome Res, 2008, 18, 610-621; Goff L A et al., PLoS One, 2009, 4:e7192; Bar M et al., Stem cells, 2008, 26, 2496-2505, the content of each of which is incorporated herein by references in its entirety).

In some embodiments, the binding sites of any of the microRNAs described herein can be incorporated into a nucleic acid of the invention (e.g., in a cleavable linker of an oligonucleotide of the invention).

In some embodiments, the nucleic acids or mRNA of the present invention includes at least one microRNA sequence in a region of the nucleic acid or mRNA which may interact with a RNA binding protein (e.g., the 3′-UTR or the 5′-UTR of an mRNA).

### Alternative Nucleotides, Nucleosides, Nucleobases, and Internucleoside Linkages

Herein, in a nucleotide, nucleoside or polynucleotide (such as the nucleic acids of the invention, e.g., an mRNA or an oligonucleotide), the terms “alteration” or, as appropriate, “alternative” refer to alteration with respect to A, G, U or C ribonucleotides. Generally, herein, these terms are not intended to refer to the ribonucleotide alterations in naturally occurring 5′-terminal mRNA cap moieties. In a polypeptide, the term “alteration” refers to an alteration as compared to the canonical set of 20 amino acids.

The alterations may be various distinct alterations. In some embodiments, where the nucleic acid is an mRNA, the coding region, the flanking regions and/or the terminal regions may contain one, two, or more (optionally different) nucleoside or nucleotide alterations. In some embodiments, an alternative polynucleotide introduced to a cell may exhibit reduced degradation in the cell, as compared to an unaltered polynucleotide.

The polynucleotides can include any useful alteration, such as to the sugar, the nucleobase, or the internucleoside linkage (e.g., to a linking phosphate/to a phosphodiester linkage/to the phosphodiester backbone). In certain embodiments, alterations (e.g., one or more alterations) are present in each of the sugar and the internucleoside linkage. Alterations according to the present invention may be alterations of ribonucleic acids (RNAs) to deoxyribonucleic acids (DNAs), e.g., the substitution of the 2′OH of the ribofuranosyl ring to 2′H, threose nucleic acids (TNAs), glycol nucleic acids (GNAs), peptide nucleic acids (PNAs), locked nucleic acids (LNAs) or hybrids thereof). Additional alterations are described herein.

In certain embodiments, it may desirable for a nucleic acid molecule introduced into the cell to be degraded intracellularly. For example, degradation of a nucleic acid molecule may be preferable if precise timing of protein production is desired. Thus, in some embodiments, the invention provides an alternative nucleic acid molecule containing a degradation domain, which is capable of being acted on in a directed manner within a cell.

The polynucleotides can optionally include other agents (e.g., RNAi-inducing agents, RNAi agents, siRNAs, shRNAs, miRNAs, antisense RNAs, ribozymes, catalytic DNA, tRNA, RNAs that induce triple helix formation, aptamers, vectors, etc.). In some embodiments, the polynucleotides may include one or more messenger RNAs (mRNAs) having one or more alternative nucleoside or nucleotides (i.e., mRNA molecules). In some embodiments, the polynucleotides may include one or more oligonucleotides having one or more alternative nucleoside or nucleotides. In some embodiments, a composition of the invention include an mRNA and/or one or more oligonucleotides having one or more alternative nucleoside or nucleotides.

Polynucleotides

According to Aduri et al (Aduri, R. et al., AMBER force field parameters for the naturally occurring modified nucleosides in RNA. Journal of Chemical Theory and Computation. 2006. 3(4):1464-75) there are 107 naturally occurring nucleosides, including 1-methyladenosine, 2-methylthio-N6-hydroxynorvalyl carbamoyladenosine, 2-methyladenosine, 2-O-ribosylphosphate adenosine, N6-methyl-N6-threonylcarbamoyladenosine, N6-acetyladenosine, N6-glycinylcarbamoyladenosine, N6-isopentenyladenosine, N6-methyladenosine, N6-threonylcarbamoyladenosine, N6,N6-dimethyladenosine, N6-(cis-hydroxyisopentenyl)adenosine, N6-hydroxynorvalylcarbamoyladenosine, 1,2-O-dimethyladenosine, N6,2-O-dimethyladenosine, 2-O-methyladenosine, N6,N6,O-2-trimethyladenosine, 2-methylthio-N6-(cis-hydroxyisopentenyl) adenosine, 2-methylthio-N6-methyladenosine, 2-methylthio-N6-isopentenyladenosine, 2-methylthio-N6-threonyl carbamoyladenosine, 2-thiocytidine, 3-methylcytidine, N4-acetylcytidine, 5-formylcytidine, N4-methylcytidine, 5-methylcytidine, 5-hydroxymethylcytidine, lysidine, N4-acetyl-2-O-methylcytidine, 5-formyl-2-O-methylcytidine, 5,2-O-dimethylcytidine, 2-O-methylcytidine, N4,2-O-dimethylcytidine, N4,N4,2-O-trimethylcytidine, 1-methylguanosine, N2,7-dimethylguanosine, N2-methylguanosine, 2-O-ribosylphosphate guanosine, 7-methylguanosine, under modified hydroxywybutosine, 7-aminomethyl-7-deazaguanosine, 7-cyano-7-deazaguanosine, N2,N2-dimethylguanosine, 4-demethylwyosine, epoxyqueuosine, hydroxywybutosine, isowyosine, N2,7,2-O-trimethylguanosine, N2,2-O-dimethylguanosine, 1,2-O-dimethylguanosine, 2-O-methylguanosine, N2,N2,2-O-trimethylguanosine, N2,N2,7-trimethylguanosine, peroxywybutosine, galactosyl-queuosine, mannosyl-queuosine, queuosine, archaeosine, wybutosine, methylwyosine, wyosine, 2-thiouridine, 3-(3-amino-3-carboxypropyl)uridine, 3-methyluridine, 4-thiouridine, 5-methyl-2-thiouridine, 5-methylaminomethyluridine, 5-carboxymethyluridine, 5-carboxymethylaminomethyluridine, 5-hydroxyuridine, 5-methyluridine, 5-taurinomethyluridine, 5-carbamoylmethyluridine, 5-(carboxyhydroxymethyl)uridine methyl ester, dihydrouridine, 5-methyldihydrouridine, 5-methylaminomethyl-2-thiouridine, 5-(carboxyhydroxymethyl)uridine, 5-(isopentenylaminomethyl)uridine, 5-(isopentenylaminomethyl)-2-thiouridine, 3,2-O-dimethyluridine, 5-carboxymethylaminomethyl-2-O-methyluridine, 5-carbamoylmethyl-2-O-methyluridine, 5-methoxycarbonylmethyl-2-O-methyluridine, 5-(isopentenylaminomethyl)-2-O-methyluridine, 5,2-O-dimethyluridine, 2-O-methyluridine, 2-thio-2-O-methyluridine, uridine 5-oxyacetic acid, 5-methoxycarbonylmethyluridine, uridine 5-oxyacetic acid methyl ester, 5-methoxyuridine, 5-aminomethyl-2-thiouridine, 5-carboxymethylaminomethyl-2-thiouridine, 5-methylaminomethyl-2-selenouridine, 5-methoxycarbonylmethyl-2-thiouridine, 5-taurinomethyl-2-thiouridine, pseudouridine, 1-methyl-3-(3-amino-3-carboxypropyl)pseudouridine, 1-methylpseudouridine, 3-methylpseudouridine, 2-O-methylpseudouridine, inosine, 1-methylinosine, 1,2-O-dimethylinosine and 2-O-methylinosine. Each of these may be components of nucleic acids of the present invention.

Alterations on the Sugar

The alternative nucleosides and nucleotides (e.g., building block molecules), which may be incorporated into a polynucleotide (e.g., RNA or mRNA, as described herein), can be altered on the sugar of the ribonucleic acid. For example, the 2′ hydroxyl group (OH) can be modified or replaced with a number of different substituents. Exemplary substitutions at the 2′-position include, but are not limited to, H, halo, optionally substituted C₁₋₆ alkyl; optionally substituted C₁₋₆ alkoxy; optionally substituted C₆₋₁₀ aryloxy; optionally substituted C₃₋₈ cycloalkyl; optionally substituted C₃₋₈ cycloalkoxy; optionally substituted C₆₋₁₀ aryloxy; optionally substituted C₆₋₁₀ aryl-C₁₋₆ alkoxy, optionally substituted C₁₋₁₂ (heterocyclyl)oxy; a sugar (e.g., ribose, pentose, or any described herein); a polyethyleneglycol (PEG), —O(CH₂CH₂O)ₙCH₂CH₂OR, where R is H or optionally substituted alkyl, and n is an integer from 0 to 20 (e.g., from 0 to 4, from 0 to 8, from 0 to 10, from 0 to 16, from 1 to 4, from 1 to 8, from 1 to 10, from 1 to 16, from 1 to 20, from 2 to 4, from 2 to 8, from 2 to 10, from 2 to 16, from 2 to 20, from 4 to 8, from 4 to 10, from 4 to 16, and from 4 to 20); “locked” nucleic acids (LNA) in which the 2′-hydroxyl is connected by a C₁₋₆ alkylene or C₁₋₆ heteroalkylene bridge to the 4′-carbon of the same ribose sugar, where exemplary bridges included methylene, propylene, ether, or amino bridges; aminoalkyl, as defined herein; aminoalkoxy, as defined herein; amino as defined herein; and amino acid, as defined herein

Generally, RNA includes the sugar group ribose, which is a 5-membered ring having an oxygen. Exemplary, non-limiting alternative nucleotides include replacement of the oxygen in ribose (e.g., with S, Se, or alkylene, such as methylene or ethylene); addition of a double bond (e.g., to replace ribose with cyclopentenyl or cyclohexenyl); ring contraction of ribose (e.g., to form a 4-membered ring of cyclobutane or oxetane); ring expansion of ribose (e.g., to form a 6- or 7-membered ring having an additional carbon or heteroatom, such as for anhydrohexitol, altritol, mannitol, cyclohexanyl, cyclohexenyl, and morpholino that also has a phosphoramidate backbone); multicyclic forms (e.g., tricyclo; and “unlocked” forms, such as glycol nucleic acid (GNA) (e.g., R-GNA or S-GNA, where ribose is replaced by glycol units attached to phosphodiester bonds), threose nucleic acid (TNA, where ribose is replace with α-L-threofuranosyl-(3′→2′)), and peptide nucleic acid (PNA, where 2-amino-ethyl-glycine linkages replace the ribose and phosphodiester backbone). The sugar group can also contain one or more carbons that possess the opposite stereochemical configuration than that of the corresponding carbon in ribose. Thus, a polynucleotide molecule can include nucleotides containing, e.g., arabinose, as the sugar.

Alterations on the Nucleobase

The present disclosure provides for alternative nucleosides and nucleotides. As described herein “nucleoside” is defined as a compound containing a sugar molecule (e.g., a pentose or ribose) or derivative thereof in combination with an organic base (e.g., a purine or pyrimidine) or a derivative thereof (also referred to herein as “nucleobase”). As described herein, “nucleotide” is defined as a nucleoside including a phosphate group.

Exemplary non-limiting alterations include an amino group, a thiol group, an alkyl group, a halo group, or any described herein. The alternative nucleotides may by synthesized by any useful method, as described herein (e.g., chemically, enzymatically, or recombinantly to include one or more alternative or alternative nucleosides).

In some embodiments, a nucleic acid of the invention (e.g., an mRNA or an oligonucleotide) includes one or more 2′-OMe nucleotides, 2′-methoxyethyl nucleotides (2′-MOE nucleotides), 2′-F nucleotide, 2′-NH2 nucleotide, 2′fluoroarabino nucleotides (FANA nucleotides), locked nucleic acid nucleotides (LNA nucleotides), or 4′-S nucleotides.

The alternative nucleotide base pairing encompasses not only the standard adenosine-thymine, adenosine-uracil, or guanosine-cytosine base pairs, but also base pairs formed between nucleotides and/or alternative nucleotides including non-standard or alternative bases, wherein the arrangement of hydrogen bond donors and hydrogen bond acceptors permits hydrogen bonding between a non-standard base and a standard base or between two complementary non-standard base structures. One example of such non-standard base pairing is the base pairing between the alternative nucleotide inosine and adenine, cytosine or uracil.

The alternative nucleosides and nucleotides can include an alternative nucleobase. Examples of nucleobases found in RNA include, but are not limited to, adenine, guanine, cytosine, and uracil. Examples of nucleobase found in DNA include, but are not limited to, adenine, guanine, cytosine, and thymine. These nucleobases can be altered or wholly replaced to provide polynucleotide molecules having enhanced properties, e.g., resistance to nucleases, stability, and these properties may manifest through disruption of the binding of a major groove binding partner.

In some embodiments, the alternative nucleobase is an alternative uracil. Exemplary nucleobases and nucleosides having an alternative uracil include pseudouridine (ψ), pyridin-4-one ribonucleoside, 5-aza-uridine, 6-aza-uridine, 2-thio-5-aza-uridine, 2-thio-uridine (s²U), 4-thio-uridine (s⁴U), 4-thio-pseudouridine, 2-thio-pseudouridine, 5-hydroxy-uridine (ho⁵U), 5-aminoallyl-uridine, 5-halo-uridine (e.g., 5-iodo-uridineor 5-bromo-uridine), 3-methyl-uridine (m³U), 5-methoxy-uridine (mo⁵U), uridine 5-oxyacetic acid (cmo⁵U), uridine 5-oxyacetic acid methyl ester (mcmo⁵U), 5-carboxymethyl-uridine (cm⁵U), 1-carboxymethyl-pseudouridine, 5-carboxyhydroxymethyl-uridine (chm⁵U), 5-carboxyhydroxymethyl-uridine methyl ester (mchm⁵U), 5-methoxycarbonylmethyl-uridine (mcm⁵U), 5-methoxycarbonylmethyl-2-thio-uridine (mcm⁵s²U), 5-aminomethyl-2-thio-uridine (nm⁵s²U), 5-methylaminomethyl-uridine (mnm⁵U), 5-methylaminomethyl-2-thio-uridine (mnm⁵s²U), 5-methylaminomethyl-2-seleno-uridine (mnm⁵se²U), 5-carbamoylmethyl-uridine (ncm⁵U), 5-carboxymethylaminomethyl-uridine (cmnm⁵U), 5-carboxymethylaminomethyl-2-thio-uridine (cmnm⁵s²U), 5-propynyl-uridine, 1-propynyl-pseudouridine, 5-taurinomethyl-uridine (τm⁵U), 1-taurinomethyl-pseudouridine, 5-taurinomethyl-2-thio-uridine(τm⁵s²U), 1-taurinomethyl-4-thio-pseudouridine, 5-methyl-uridine (m⁵U, i.e., having the nucleobase deoxythymine), 1-methyl-pseudouridine 5-methyl-2-thio-uridine (m⁵s²U), 1-methyl-4-thio-pseudouridine (m¹s⁴ψ), 4-thio-1-methyl-pseudouridine, 3-methyl-pseudouridine (m³ψ), 2-thio-1-methyl-pseudouridine, 1-methyl-1-deaza-pseudouridine, 2-thio-1-methyl-1-deaza-pseudouridine, dihydrouridine (D), dihydropseudouridine, 5,6-dihydrouridine, 5-methyl-dihydrouridine (m⁵D), 2-thio-dihydrouridine, 2-thio-dihydropseudouridine, 2-methoxy-uridine, 2-methoxy-4-thio-uridine, 4-methoxy-pseudouridine, 4-methoxy-2-thio-pseudouridine, N1-methyl-pseudouridine, 3-(3-amino-3-carboxypropyl)uridine (acp³U), 1-methyl-3-(3-amino-3-carboxypropyl)pseudouridine (acp³ ψ), 5-(isopentenylaminomethyl)uridine (inm⁵U), 5-(isopentenylaminomethyl)-2-thio-uridine (inm⁵s²U), α-thio-uridine, 2′-O-methyl-uridine (Um), 5,2′-O-dimethyl-uridine (m⁵Um), 2′-O-methyl-pseudouridine 2-thio-2′-O-methyl-uridine (s²Um), 5-methoxycarbonylmethyl-2′-O-methyl-uridine (mcm⁵Um), 5-carbamoyl methyl-2′-O-methyl-uridine (ncm⁵Um), 5-carboxymethylaminomethyl-2′-O-methyl-uridine (cmnm⁵Um), 3,2′-O-dimethyl-uridine (m³Um), and 5-(isopentenylaminomethyl)-2′-O-methyl-uridine (inm⁵Um), 1-thio-uridine, deoxythymidine, 2′-F-ara-uridine, 2′-F-uridine, 2′-OH-ara-uridine, 5-(2-carbomethoxyvinyl) uridine, and 5-[3-(1-E-propenylamino)uridine.

In some embodiments, the alternative nucleobase is an alternative cytosine. Exemplary nucleobases and nucleosides having an alternative cytosine include 5-aza-cytidine, 6-aza-cytidine, pseudoisocytidine, 3-methyl-cytidine (m³C), N4-acetyl-cytidine (ac⁴C), 5-formyl-cytidine (f⁵C), N4-methyl-cytidine (m⁴C), 5-methyl-cytidine (m⁵C), 5-halo-cytidine (e.g., 5-iodo-cytidine), 5-hydroxymethyl-cytidine (hm⁵C), 1-methyl-pseudoisocytidine, pyrrolo-cytidine, pyrrolo-pseudoisocytidine, 2-thio-cytidine (s²C), 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, 4-methoxy-1-methyl-pseudoisocytidine, lysidine (k₂C), α-thio-cytidine, 2′-O-methyl-cytidine (Cm), 5,2′-O-dimethyl-cytidine (m⁵Cm), N4-acetyl-2′-O-methyl-cytidine (ac⁴Cm), N4,2′-O-dimethyl-cytidine (m⁴Cm), 5-formyl-2′-O-methyl-cytidine (f⁵Cm), N4,N4,2′-O-trimethyl-cytidine (m⁴2 Cm), 1-thio-cytidine, 2′-F-ara-cytidine, 2′-F-cytidine, and 2′-OH-ara-cytidine.

In some embodiments, the alternative nucleobase is an alternative adenine. Exemplary nucleobases and nucleosides having an alternative adenine include 2-amino-purine, 2, 6-diaminopurine, 2-amino-6-halo-purine (e.g., 2-amino-6-chloro-purine), 6-halo-purine (e.g., 6-chloro-purine), 2-amino-6-methyl-purine, 8-azido-adenosine, 7-deaza-adenine, 7-deaza-8-aza-adenine, 7-deaza-2-amino-purine, 7-deaza-8-aza-2-amino-purine, 7-deaza-2,6-diaminopurine, 7-deaza-8-aza-2,6-diaminopurine, 1-methyl-adenosine (m¹A), 2-methyl-adenine (m²A), N6-methyl-adenosine (m⁶A), 2-methylthio-N6-methyl-adenosine (ms² m⁶A), N6-isopentenyl-adenosine (i⁶A), 2-methylthio-N6-isopentenyl-adenosine (ms²i⁶A), N6-(cis-hydroxyisopentenyl)adenosine (io⁶A), 2-methylthio-N6-(cis-hydroxyisopentenyl)adenosine (ms²io⁶A), N6-glycinylcarbamoyl-adenosine (g⁶A), N6-threonylcarbamoyl-adenosine (t⁶A), N6-methyl-N6-threonylcarbamoyl-adenosine (m⁶t⁶A), 2-methylthio-N6-threonylcarbamoyl-adenosine (ms²g⁶A), N6,N6-dimethyl-adenosine (m⁶₂A), N6-hydroxynorvalylcarbamoyl-adenosine (hn⁶A), 2-methylthio-N6-hydroxynorvalylcarbamoyl-adenosine (ms²hn⁶A), N6-acetyl-adenosine (ac⁶A), 7-methyl-adenine, 2-methylthio-adenine, 2-methoxy-adenine, α-thio-adenosine, 2′-O-methyl-adenosine (Am), N6,2′-O-dimethyl-adenosine (m⁶Am), N6,N6,2′-O-trimethyl-adenosine (m⁶₂Am), 1,2′-O-dimethyl-adenosine (m¹Am), 2′-O-ribosyladenosine (phosphate) (Ar(p)), 2-amino-N6-methyl-purine, 1-thio-adenosine, 8-azido-adenosine, 2′-F-ara-adenosine, 2′-F-adenosine, 2′-OH-ara-adenosine, and N6-(19-amino-pentaoxanonadecyl)-adenosine.

In some embodiments, the alternative nucleobase is an alternative guanine. Exemplary nucleobases and nucleosides having an alternative guanine include inosine (I), 1-methyl-inosine (m¹I), wyosine (imG), methylwyosine (mimG), 4-demethyl-wyosine (imG-14), isowyosine (imG2), wybutosine (yW), peroxywybutosine (o₂yW), hydroxywybutosine (OhyW), undermodified hydroxywybutosine (OhyW*), 7-deaza-guanosine, queuosine (Q), epoxyqueuosine (oQ), galactosyl-queuosine (galQ), mannosyl-queuosine (manQ), 7-cyano-7-deaza-guanosine (preQ₀), 7-aminomethyl-7-deaza-guanosine (preQ₁), archaeosine (G⁺), 7-deaza-8-aza-guanosine, 6-thio-guanosine, 6-thio-7-deaza-guanosine, 6-thio-7-deaza-8-aza-guanosine, 7-methyl-guanosine (m⁷G), 6-thio-7-methyl-guanosine, 7-methyl-inosine, 6-methoxy-guanosine, 1-methyl-guanosine (m¹G), N2-methyl-guanosine (m²G), N2,N2-dimethyl-guanosine (m²₂G), N2,7-dimethyl-guanosine (m^{2,7}G), N2, N2,7-dimethyl-guanosine (m^{2,2,7}G), 8-oxo-guanosine, 7-methyl-8-oxo-guanosine, 1-methyl-6-thio-guanosine, N2-methyl-6-thio-guanosine, N2,N2-dimethyl-6-thio-guanosine, α-thio-guanosine, 2′-O-methyl-guanosine (Gm), N2-methyl-2′-O-methyl-guanosine (m²Gm), N2,N2-dimethyl-2′-O-methyl-guanosine (m²₂Gm), 1-methyl-2′-O-methyl-guanosine (m¹Gm), N2,7-dimethyl-2′-O-methyl-guanosine (m^{2,7}Gm), 2′-O-methyl-inosine (Im), 1,2′-O-dimethyl-inosine (m¹Im), 2′-O-ribosylguanosine (phosphate) (Gr(p)), 1-thio-guanosine, 06-methyl-guanosine, 2′-F-ara-guanosine, and 2′-F-guanosine.

The nucleobase of the nucleotide can be independently selected from a purine, a pyrimidine, a purine or pyrimidine analog. For example, the nucleobase can each be independently selected from adenine, cytosine, guanine, uracil, or hypoxanthine. In some embodiments, the nucleobase can also include, for example, naturally-occurring and synthetic derivatives of a base, including pyrazolo[3,4-d]pyrimidines, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-propynyl uracil and cytosine, 6-azo uracil, cytosine and thymine, 5-uracil (pseudouracil), 4-thiouracil, 8-halo (e.g., 8-bromo), 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, deazaguanine, 7-deazaguanine, 3-deazaguanine, deazaadenine, 7-deazaadenine, 3-deazaadenine, pyrazolo[3,4-d]pyrimidine, imidazo[1,5-a]1,3,5 triazinones, 9-deazapurines, imidazo[4,5-d]pyrazines, thiazolo[4,5-d]pyrimidines, pyrazin-2-ones, 1,2,4-triazine, pyridazine; and 1,3,5 triazine. When the nucleotides are depicted using the shorthand A, G, C, T or U, each letter refers to the representative base and/or derivatives thereof, e.g., A includes adenine or adenine analogs, e.g., 7-deaza adenine).

In some embodiments, the polynucleotides of the invention contain 5-methoxy-uracil, uracil, 5-methyl-cytosine, and cytosine as the only uracils and cytosines. In some embodiments, the polynucleotides of the invention contain 5-methoxy-uracil, uracil, 5-trifluoromethyl-cytosine, and cytosine as the only uracils and cytosines. In some embodiments, the polynucleotides of the invention contain 5-methoxy-uracil, uracil, 5-hydroxymethyl-cytosine, and cytosine as the only uracils and cytosines. In some embodiments, the polynucleotides of the invention contain 5-methoxy-uracil, uracil, 5-bromo-cytosine, and cytosine as the only uracils and cytosines. In some embodiments, the polynucleotides of the invention contain 5-methoxy-uracil, uracil, 5-iodo-cytosine, and cytosine as the only uracils and cytosines. In some embodiments, the polynucleotides of the invention contain 5-methoxy-uracil, uracil, 5-methoxy-cytosine, and cytosine as the only uracils and cytosines. In some embodiments, the polynucleotides of the invention contain 5-methoxy-uracil, uracil, 5-ethyl-cytosine, and cytosine as the only uracils and cytosines. In some embodiments, the polynucleotides of the invention contain 5-methoxy-uracil, uracil, 5-phenyl-cytosine, and cytosine as the only uracils and cytosines. In some embodiments, the polynucleotides of the invention contain 5-methoxy-uracil, uracil, 5-ethnyl-cytosine, and cytosine as the only uracils and cytosines. In some embodiments, the polynucleotides of the invention contain 5-methoxy-uracil, uracil, N4-methyl-cytosine, and cytosine as the only uracils and cytosines. In some embodiments, the polynucleotides of the invention contain 5-methoxy-uracil, uracil, 5-fluoro-cytosine, and cytosine as the only uracils and cytosines. In some embodiments, the polynucleotides of the invention contain 5-methoxy-uracil, uracil, N4-acetyl-cytosine, and cytosine as the only uracils and cytosines. In some embodiments, the polynucleotides of the invention contain 5-methoxy-uracil, uracil, pseudoisocytosine, and cytosine as the only uracils and cytosines. In some embodiments, the polynucleotides of the invention contain 5-methoxy-uracil, uracil, 5-formyl-cytosine, and cytosine as the only uracils and cytosines. In some embodiments, the polynucleotides of the invention contain 5-methoxy-uracil, uracil, 5-aminoallyl-cytosine, and cytosine as the only uracils and cytosines. In some embodiments, the polynucleotides of the invention contain 5-methoxy-uracil, uracil, 5-carboxy-cytosine, and cytosine as the only uracils and cytosines.

In some embodiments, the polynucleotides of the invention contain 1-methyl-pseudouracil, uracil, 5-methyl-cytosine, and cytosine as the only uracils and cytosines. In some embodiments, the polynucleotides of the invention contain 1-methyl-pseudouracil, uracil, 5-trifluoromethyl-cytosine, and cytosine as the only uracils and cytosines. In some embodiments, the polynucleotides of the invention contain 1-methyl-pseudouracil, uracil, 5-hydroxymethyl-cytosine, and cytosine as the only uracils and cytosines. In some embodiments, the polynucleotides of the invention contain 1-methyl-pseudouracil, uracil, 5-bromo-cytosine, and cytosine as the only uracils and cytosines. In some embodiments, the polynucleotides of the invention contain 1-methyl-pseudouracil, uracil, 5-iodo-cytosine, and cytosine as the only uracils and cytosines. In some embodiments, the polynucleotides of the invention contain 1-methyl-pseudouracil, uracil, 5-methoxy-cytosine, and cytosine as the only uracils and cytosines. In some embodiments, the polynucleotides of the invention contain 1-methyl-pseudouracil, uracil, 5-ethyl-cytosine, and cytosine as the only uracils and cytosines. In some embodiments, the polynucleotides of the invention contain 1-methyl-pseudouracil, uracil, 5-phenyl-cytosine, and cytosine as the only uracils and cytosines. In some embodiments, the polynucleotides of the invention contain 1-methyl-pseudouracil, uracil, 5-ethnyl-cytosine, and cytosine as the only uracils and cytosines. In some embodiments, the polynucleotides of the invention contain 1-methyl-pseudouracil, uracil, N4-methyl-cytosine, and cytosine as the only uracils and cytosines. In some embodiments, the polynucleotides of the invention contain 1-methyl-pseudouracil, uracil, 5-fluoro-cytosine, and cytosine as the only uracils and cytosines. In some embodiments, the polynucleotides of the invention contain 1-methyl-pseudouracil, uracil, N4-acetyl-cytosine, and cytosine as the only uracils and cytosines. In some embodiments, the polynucleotides of the invention contain 1-methyl-pseudouracil, uracil, pseudoisocytosine, and cytosine as the only uracils and cytosines. In some embodiments, the polynucleotides of the invention contain 1-methyl-pseudouracil, uracil, 5-formyl-cytosine, and cytosine as the only uracils and cytosines. In some embodiments, the polynucleotides of the invention contain 1-methyl-pseudouracil, uracil, 5-aminoallyl-cytosine, and cytosine as the only uracils and cytosines. In some embodiments, the polynucleotides of the invention contain 1-methyl-pseudouracil, uracil, 5-carboxy-cytosine, and cytosine as the only uracils and cytosines.

In some embodiments, the polynucleotides of the invention contain 5-methoxy-uridine, uridine, 5-methyl-cytidine, and cytidine as the only uridines and cytidines. In some embodiments, the polynucleotides of the invention contain 5-methoxy-uridine, uridine, 5-trifluoromethyl-cytidine, and cytidine as the only uridines and cytidines. In some embodiments, the polynucleotides of the invention contain 5-methoxy-uridine, uridine, 5-hydroxymethyl-cytidine, and cytidine as the only uridines and cytidines. In some embodiments, the polynucleotides of the invention contain 5-methoxy-uridine, uridine, 5-bromo-cytidine, and cytidine as the only uridines and cytidines. In some embodiments, the polynucleotides of the invention contain 5-methoxy-uridine, uridine, 5-iodo-cytidine, and cytidine as the only uridines and cytidines. In some embodiments, the polynucleotides of the invention contain 5-methoxy-uridine, uridine, 5-methoxy-cytidine, and cytidine as the only uridines and cytidines. In some embodiments, the polynucleotides of the invention contain 5-methoxy-uridine, uridine, 5-ethyl-cytidine, and cytidine as the only uridines and cytidines. In some embodiments, the polynucleotides of the invention contain 5-methoxy-uridine, uridine, 5-phenyl-cytidine, and cytidine as the only uridines and cytidines. In some embodiments, the polynucleotides of the invention contain 5-methoxy-uridine, uridine, 5-ethnyl-cytidine, and cytidine as the only uridines and cytidines. In some embodiments, the polynucleotides of the invention contain 5-methoxy-uridine, uridine, N4-methyl-cytidine, and cytidine as the only uridines and cytidines. In some embodiments, the polynucleotides of the invention contain 5-methoxy-uridine, uridine, 5-fluoro-cytidine, and cytidine as the only uridines and cytidines. In some embodiments, the polynucleotides of the invention contain 5-methoxy-uridine, uridine, N4-acetyl-cytidine, and cytidine as the only uridines and cytidines. In some embodiments, the polynucleotides of the invention contain 5-methoxy-uridine, uridine, pseudoisocytidine, and cytidine as the only uridines and cytidines. In some embodiments, the polynucleotides of the invention contain 5-methoxy-uridine, uridine, 5-formyl-cytidine, and cytidine as the only uridines and cytidines. In some embodiments, the polynucleotides of the invention contain 5-methoxy-uridine, uridine, 5-aminoallyl-cytidine, and cytidine as the only uridines and cytidines. In some embodiments, the polynucleotides of the invention contain 5-methoxy-uridine, uridine, 5-carboxy-cytidine, and cytidine as the only uridines and cytidines.

In some embodiments, the polynucleotides of the invention contain 1-methyl-pseudouridine, uridine, 5-methyl-cytidine, and cytidine as the only uridines and cytidines. In some embodiments, the polynucleotides of the invention contain 1-methyl-pseudouridine, uridine, 5-trifluoromethyl-cytidine, and cytidine as the only uridines and cytidines. In some embodiments, the polynucleotides of the invention contain 1-methyl-pseudouridine, uridine, 5-hydroxymethyl-cytidine, and cytidine as the only uridines and cytidines. In some embodiments, the polynucleotides of the invention contain 1-methyl-pseudouridine, uridine, 5-bromo-cytidine, and cytidine as the only uridines and cytidines. In some embodiments, the polynucleotides of the invention contain 1-methyl-pseudouridine, uridine, 5-iodo-cytidine, and cytidine as the only uridines and cytidines. In some embodiments, the polynucleotides of the invention contain 1-methyl-pseudouridine, uridine, 5-methoxy-cytidine, and cytidine as the only uridines and cytidines. In some embodiments, the polynucleotides of the invention contain 1-methyl-pseudouridine, uridine, 5-ethyl-cytidine, and cytidine as the only uridines and cytidines. In some embodiments, the polynucleotides of the invention contain 1-methyl-pseudouridine, uridine, 5-phenyl-cytidine, and cytidine as the only uridines and cytidines. In some embodiments, the polynucleotides of the invention contain 1-methyl-pseudouridine, uridine, 5-ethnyl-cytidine, and cytidine as the only uridines and cytidines. In some embodiments, the polynucleotides of the invention contain 1-methyl-pseudouridine, uridine, N4-methyl-cytidine, and cytidine as the only uridines and cytidines. In some embodiments, the polynucleotides of the invention contain 1-methyl-pseudouridine, uridine, 5-fluoro-cytidine, and cytidine as the only uridines and cytidines. In some embodiments, the polynucleotides of the invention contain 1-methyl-pseudouridine, uridine, N4-acetyl-cytidine, and cytidine as the only uridines and cytidines. In some embodiments, the polynucleotides of the invention contain 1-methyl-pseudouridine, uridine, pseudoisocytidine, and cytidine as the only uridines and cytidines. In some embodiments, the polynucleotides of the invention contain 1-methyl-pseudouridine, uridine, 5-formyl-cytidine, and cytidine as the only uridines and cytidines. In some embodiments, the polynucleotides of the invention contain 1-methyl-pseudouridine, uridine, 5-aminoallyl-cytidine, and cytidine as the only uridines and cytidines. In some embodiments, the polynucleotides of the invention contain 1-methyl-pseudouridine, uridine, 5-carboxy-cytidine, and cytidine as the only uridines and cytidines.

In some embodiments, the polynucleotides of the invention contain the uracil of one of the nucleosides of Table 1 and uracil as the only uracils. In other embodiments, the polynucleotides of the invention contain a uridine of Table 1 and uridine as the only uridines.

TABLE 1
Exemplary uracil containing nucleosides
Nucleoside Name

5-methoxy-uridine
1-Methyl-pseudo-uridine
pseudouridine
5-methyl-uridine
5-bromo-uridine
2-thio-uridine
4-thiouridine
2′-O-methyluridine
5-methyl-2-thiouridine
5,2′-O-dimethyluridine
5-aminomethyl-2-thiouridine
5-(1-Propynyl)ara-uridine
2′-O-Methyl-5-(1-propynyl)uridine
5-Vinylarauridine
(Z)-5-(2-Bromo-vinyl)ara-uridine
(E)-5-(2-Bromo-vinyl)ara-uridine
(Z)-5-(2-Bromo-vinyl)uridine
(E)-5-(2-Bromo-vinyl)uridine
5-Cyanouridine
5-Formyluridine
5-Dimethylaminouridine
5-Trideuteromethyl-6-deuterouridine
5-(2-Furanyl)uridine
5-Phenylethynyluridine
4′-Carbocyclic uridine
4′-Ethynyluridine
4′-Azidouridine
2′-Deoxy-2′,2′-difluorouridine
2′-Deoxy-2′-b-fluorouridine
2′-Deoxy-2′-b-chlorouridine
2′-Deoxy-2′-b-bromouridine
2′-Deoxy-2′-b-iodouridine
5′-Homo-uridine
2′-Deoxy-2′-a-mercaptouridine
2′-Deoxy-2′-a-thiomethoxyuridine
2′-Deoxy-2′-a-azidouridine
2′-Deoxy-2′-a-aminouridine
2′-Deoxy-2′-b-mercaptouridine
2′-Deoxy-2′-b-thiomethoxyuridine
2′-Deoxy-2′-b-azidouridine
2′-Deoxy-2′-b-aminouridine
2′-b-Trifluoromethyluridine
2′-a-Trifluoromethyluridine
2′-b-Ethynyluridine
2′-a-Ethynyluridine
1-ethyl-pseudo-uridine
1-propyl-pseudo-uridine
1-iso-propyl-pseudo-uridine
1-(2,2,2-trifluoroethyl)-pseudo-uridine
1-cyclopropyl-pseudo-uridine
1-cyclopropylmethyl-pseudo-uridine
1-phenyl-pseudo-uridine
1-benzyl-pseudo-uridine
1-aminomethyl-pseudo-uridine
pseudo-uridine-1-2-ethanoic acid
1-(3-amino-3-carboxypropyl)pseudo-uridine
1-methyl-3-(3-amino-3-carboxypropyl)pseudo-uridine
6-methyl-pseudo-uridine
6-trifluoromethyl-pseudo-uridine
6-methoxy-pseudo-uridine
6-phenyl-pseudo-uridine
6-iodo-pseudo-uridine
6-bromo-pseudo-uridine
6-chloro-pseudo-uridine
6-fluoro-pseudo-uridine
4-Thio-pseudo-uridine
2-Thio-pseudo-uridine
Alpha-thio-pseudo-uridine
1-Me-alpha-thio-pseudo-uridine
1-butyl-pseudo-uridine
1-tert-butyl-pseudo-uridine
1-pentyl-pseudo-uridine
1-hexyl-pseudo-uridine
1-trifluoromethyl-pseudo-uridine
1-cyclobutyl-pseudo-uridine
1-cyclopentyl-pseudo-uridine
1-cyclohexyl-pseudo-uridine
1-cycloheptyl-pseudo-uridine
1-cyclooctyl-pseudo-uridine
1-cyclobutylmethyl-pseudo-uridine
1-cyclopentylmethyl-pseudo-uridine
1-cyclohexylmethyl-pseudo-uridine
1-cycloheptylmethyl-pseudo-uridine
1-cyclooctylmethyl-pseudo-uridine
1-p-tolyl-pseudo-uridine
1-(2,4,6-trimethyl-phenyl)pseudo-uridine
1-(4-methoxy-phenyl)pseudo-uridine
1-(4-amino-phenyl)pseudo-uridine
1(4-nitro-phenyl)pseudo-uridine
pseudo-uridine-N1-p-benzoic acid
1-(4-methyl-benzyl)pseudo-uridine
1-(2,4,6-trimethyl-benzyl)pseudo-uridine
1-(4-methoxy-benzyl)pseudo-uridine
1-(4-amino-benzyl)pseudo-uridine
1-(4-nitro-benzyl)pseudo-uridine
pseudo-uridine-N1-methyl-p-benzoic acid
1-(2-amino-ethyl)pseudo-uridine
1-(3-amino-propyl)pseudo-uridine
1-(4-amino-butyl)pseudo-uridine
1-(5-amino-pentyl)pseudo-uridine
1-(6-amino-hexyl)pseudo-uridine
pseudo-uridine-N1-3-propionic acid
pseudo-uridine-N1-4-butanoic acid
pseudo-uridine-N1-5-pentanoic acid
pseudo-uridine-N1-6-hexanoic acid
pseudo-uridine-N1-7-heptanoic acid
1-(2-amino-2-carboxyethyl)pseudo-uridine
1-(4-amino-4-carboxybutyl)pseudo-uridine
3-alkyl-pseudo-uridine
6-ethyl-pseudo-uridine
6-propyl-pseudo-uridine
6-iso-propyl-pseudo-uridine
6-butyl-pseudo-uridine
6-tert-butyl-pseudo-uridine
6-(2,2,2-trifluoroethyl)-pseudo-uridine
6-ethoxy-pseudo-uridine
6-trifluoromethoxy-pseudo-uridine
6-phenyl-pseudo-uridine
6-(substituted-phenyl)-pseudo-uridine
6-cyano-pseudo-uridine
6-azido-pseudo-uridine
6-amino-pseudo-uridine
6-ethylcarboxylate-pseudo-uridine
6-hydroxy-pseudo-uridine
6-methylamino-pseudo-uridine
6-dimethylamino-pseudo-uridine
6-hydroxyamino-pseudo-uridine
6-formyl-pseudo-uridine
6-(4-morpholino)-pseudo-uridine
6-(4-thiomorpholino)-pseudo-uridine
1-me-4-thio-pseudo-uridine
1-me-2-thio-pseudo-uridine
1,6-dimethyl-pseudo-uridine
1-methyl-6-trifluoromethyl-pseudo-uridine
1-methyl-6-ethyl-pseudo-uridine
1-methyl-6-propyl-pseudo-uridine
1-methyl-6-iso-propyl-pseudo-uridine
1-methyl-6-butyl-pseudo-uridine
1-methyl-6-tert-butyl-pseudo-uridine
1-methyl-6-(2,2,2-trifluoroethyl)pseudo-uridine
1-methyl-6-iodo-pseudo-uridine
1-methyl-6-bromo-pseudo-uridine
1-methyl-6-chloro-pseudo-uridine
1-methyl-6-fluoro-pseudo-uridine
1-methyl-6-methoxy-pseudo-uridine
1-methyl-6-ethoxy-pseudo-uridine
1-methyl-6-trifluoromethoxy-pseudo-uridine
1-methyl-6-phenyl-pseudo-uridine
1-methyl-6-(substituted phenyl)pseudo-uridine
1-methyl-6-cyano-pseudo-uridine
1-methyl-6-azido-pseudo-uridine
1-methyl-6-amino-pseudo-uridine
1-methyl-6-ethylcarboxylate-pseudo-uridine
1-methyl-6-hydroxy-pseudo-uridine
1-methyl-6-methylamino-pseudo-uridine
1-methyl-6-dimethylamino-pseudo-uridine
1-methyl-6-hydroxyamino-pseudo-uridine
1-methyl-6-formyl-pseudo-uridine
1-methyl-6-(4-morpholino)-pseudo-uridine
1-methyl-6-(4-thiomorpholino)-pseudo-uridine
1-alkyl-6-vinyl-pseudo-uridine
1-alkyl-6-allyl-pseudo-uridine
1-alkyl-6-homoallyl-pseudo-uridine
1-alkyl-6-ethynyl-pseudo-uridine
1-alkyl-6-(2-propynyl)-pseudo-uridine
1-alkyl-6-(1-propynyl)-pseudo-uridine
1-Hydroxymethylpseudouridine
1-(2-Hydroxyethyl)pseudouridine
1-Methoxymethylpseudouridine
1-(2-Methoxyethyl)pseudouridine
1-(2,2-Diethoxyethyl)pseudouridine
(±)1-(2-Hydroxypropyl)pseudouridine
(2R)-1-(2-Hydroxypropyl)pseudouridine
(2S)-1-(2-Hydroxypropyl)pseudouridine
1-Cyanomethylpseudouridine
1-Morpholinomethylpseudouridine
1-Thiomorpholinomethylpseudouridine
1-Benzyloxymethylpseudouridine
1-(2,2,3,3,3-Pentafluoropropyl)pseudouridine
1-Thiomethoxymethylpseudouridine
1-Methanesulfonylmethylpseudouridine
1-Vinylpseudouridine
1-Allylpseudouridine
1-Homoallylpseudouridine
1-Propargylpseudouridine
1-(4-Fluorobenzyl)pseudouridine
1-(4-Chlorobenzyl)pseudouridine
1-(4-Bromobenzyl)pseudouridine
1-(4-Iodobenzyl)pseudouridine
1-(4-Methylbenzyl)pseudouridine
1-(4-Trifluoromethylbenzyl)pseudouridine
1-(4-Methoxybenzyl)pseudouridine
1-(4-Trifluoromethoxybenzyl)pseudouridine
1-(4-Thiomethoxybenzyl)pseudouridine
1-(4-Methanesulfonylbenzyl)pseudouridine
Pseudouridine 1-(4-methylbenzoic acid)
Pseudouridine 1-(4-methylbenzenesulfonic acid)
1-(2,4,6-Trimethylbenzyl)pseudouridine
1-(4-Nitrobenzyl)pseudouridine
1-(4-Azidobenzyl)pseudouridine
1-(3,4-Dimethoxybenzyl)pseudouridine
1-(3,4-Bis-trifluoromethoxybenzyl)pseudouridine
1-Acetylpseudouridine
1-Trifluoroacetylpseudouridine
1-Benzoylpseudouridine
1-Pivaloylpseudouridine
1-(3-Cyclopropyl-prop-2-ynyl)pseudouridine
Pseudouridine 1-methylphosphonic acid diethyl ester
Pseudouridine 1-methylphosphonic acid
Pseudouridine 1-[3-(2-ethoxy)]propionic acid
Pseudouridine 1-[3-{2-(2-ethoxy)-ethoxy}] propionic acid
Pseudouridine TP 1-[3-{2-(2-[2-ethoxy]-ethoxy)-ethoxy}]
propionic acid
Pseudouridine 1-[3-{2-(2-[2-(2-ethoxy)-ethoxy]-ethoxy)-
ethoxy}]propionic acid
Pseudouridine 1-[3-{2-(2-[2-{2(2-ethoxy)-ethoxy}-ethoxy]-
ethoxy)-ethoxy}]propionic acid
1-{3-[2-(2-Aminoethoxy)-ethoxy]-propionyl} pseudouridine
1-[3-(2-{2-[2-(2-Aminoethoxy)-ethoxy]-ethoxy}-ethoxy)-
propionyl]pseudouridine
1-Biotinylpseudouridine
1-Biotinyl-PEG2-pseudouridine
5-Oxyacetic acid-methyl ester-uridine
3-Methyl-pseudo-uridine
5-trifluoromethyl-uridine
5-methyl-amino-methyl-uridine
5-carboxy-methyl-amino-methyl-uridine
5-carboxymethylaminomethyl-2′-OMe-uridine
5-carboxymethylaminomethyl-2-thio-uridine
5-methylaminomethyl-2-thio-uridine
5-methoxy-carbonyl-methyl-uridine
5-methoxy-carbonyl-methyl-2′-OMe-uridine
5-oxyacetic acid-uridine
3-(3-amino-3-carboxypropyl)-uridine
5-(carboxyhydroxymethyl)uridine methyl ester
5-(carboxyhydroxymethyl)uridine
2′-OMe-pseudo-uridine
2′-Azido-2′-deoxy-uridine
2′-Amino-2′-deoxy-uridine
2′-F-5-Methyl-2′-deoxy-uridine
5-iodo-2′-fluoro-deoxyuridine
2′-bromo-deoxyuridine
2,2′-anhydro-uridine
2′-Azido-deoxyuridine
5-Methoxycarbonylmethyl-2-thiouridine
5-Methylaminomethyl-2-thiouridine
5-Carbamoylmethyluridine
5-Carbamoylmethyl-2′-O-methyluridine
1-Methyl-3-(3-amino-3-carboxypropyl) pseudouridine
5-Methylaminomethyl-2-selenouridine
5-Carboxymethyluridine
5-Methyldihydrouridine
5-Taurinomethyluridine
5-Taurinomethyl-2-thiouridine
5-(iso-Pentenylaminomethyl)uridine
5-(iso-Pentenylaminomethyl)-2-thiouridine
5-(iso-Pentenylaminomethyl)-2′-O-methyluridine
2′-O-Methylpseudouridine
2-Thio-2′-O-methyluridine
3,2′-O-Dimethyluridine
5-Methoxy-carbonylmethyl-uridine
5-hydroxy-uridine
5-Isopentenyl-aminomethyl-uridine

In some embodiments, the polynucleotides of the invention contain the cytosine of one of the nucleosides of Table 2 and cytosine as the only cytosines. In other embodiments, the polynucleotides of the invention contain a cytidine of Table 2 and cytidine as the only cytidines.

TABLE 2
Exemplary cytosine containing nucleosides
Nucleoside Name

α-thio-cytidine
pseudoisocytidine
pyrrolo-cytidine
5-methyl-cytidine
N4-acetyl-cytidine
5-Bromo-cytidine
5-Trifluoromethyl-cytidine
5-Hydroxymethyl-cytidine
5-Iodo-cytidine
5-Ethyl-cytidine
5-Methoxy-cytidine
5-Ethynyl-cytidine
5-Fluoro-cytidine
5-Phenyl-cytidine
N4-Bz-cytidine
N4-Methyl-cytidine
5-Pseudo-iso-cytidine
5-Formyl-cytidine
5-Aminoallyl-cytidine
2′-O-methylcytidine
2′-O-Methyl-5-(1-propynyl)cytidine
5-(1-Propynyl)ara-cytidine
5-Ethynylara-cytidine
5-Ethynylcytidine
5-Cyanocytidine
5-(2-Chloro-phenyl)-2-thiocytidine
5-(4-Amino-phenyl)-2-thiocytidine
N4,2′-O-Dimethylcytidine
3′-Ethynylcytidine
4′-Carbocyclic cytidine
4′-Ethynylcytidine
4′-Azidocytidine
2′-Deoxy-2′,2′-difluorocytidine
2′-Deoxy-2′-b-fluorocytidine
2′-Deoxy-2′-b-chlorocytidine
2′-Deoxy-2′-b-bromocytidine
2′-Deoxy-2′-b-iodocytidine
5′-Homo-cytidine
2′-Deoxy-2′-a-mercaptocytidine
2′-Deoxy-2′-a-thiomethoxycytidine
2′-Deoxy-2′-a-azidocytidine
2′-Deoxy-2′-a-aminocytidine
2′-Deoxy-2′-b-mercaptocytidine
2′-Deoxy-2′-b-thiomethoxycytidine
2′-Deoxy-2′-b-azidocytidine
2′-Deoxy-2′-b-aminocytidine
2′-b-Trifluoromethylcytidine
2′-a-Trifluoromethylcytidine
2′-b-Ethynylcytidine
2′-a-Ethynylcytidine
(E)-5-(2-Bromo-vinyl)cytidine
2′-Azido-2′-deoxy-cytidine
2′-Amino-2′-deoxy-cytidine
5-aminoallyl-cytidine
2,2′-anhydro-cytidine
N4-amino-cytidine
2′-O-Methyl-N4-acetyl-cytidine
2′-fluoro-N4-acetyl-cytidine
2′-fluor-N4-Bz-cytidine
2′-O-methyl-N4-Bz-cytidine
N4,2′-O-Dimethylcytidine
5-Formyl-2′-O-methylcytidine

Alterations on the Internucleoside Linkage

The alternative nucleotides, which may be incorporated into a polynucleotide molecule, can be altered on the internucleoside linkage (e.g., phosphate backbone). Herein, in the context of the polynucleotide backbone, the phrases “phosphate” and “phosphodiester” are used interchangeably. Backbone phosphate groups can be altered by replacing one or more of the oxygen atoms with a different substituent.

The alternative nucleosides and nucleotides can include the wholesale replacement of an unaltered phosphate moiety with another internucleoside linkage as described herein. Examples of alternative phosphate groups include, but are not limited to, phosphorothioate, phosphoroselenates, boranophosphates, boranophosphate esters, hydrogen phosphonates, phosphoramidates, phosphorodiamidates, alkyl or aryl phosphonates, and phosphotriesters. Phosphorodithioates have both non-linking oxygens replaced by sulfur. The phosphate linker can also be altered by the replacement of a linking oxygen with nitrogen (bridged phosphoramidates), sulfur (bridged phosphorothioates), and carbon (bridged methylene-phosphonates).

The alternative nucleosides and nucleotides can include the replacement of one or more of the non-bridging oxygens with a borane moiety (BH₃), sulfur (thio), methyl, ethyl and/or methoxy. As a non-limiting example, two non-bridging oxygens at the same position (e.g., the alpha (a), beta (β) or gamma (γ) position) can be replaced with a sulfur (thio) and a methoxy.

The replacement of one or more of the oxygen atoms at the a position of the phosphate moiety (e.g., α-thio phosphate) is provided to confer stability (such as against exonucleases and endonucleases) to RNA and DNA through the unnatural phosphorothioate backbone linkages. Phosphorothioate DNA and RNA have increased nuclease resistance and subsequently a longer half-life in a cellular environment. While not wishing to be bound by theory, phosphorothioate linked polynucleotide molecules are expected to also reduce the innate immune response through weaker binding/activation of cellular innate immune molecules.

In specific embodiments, an alternative nucleoside includes an alpha-thio-nucleoside (e.g., 5′-O-(1-thiophosphate)-adenosine, 5′-O-(1-thiophosphate)-cytidine (α-thio-cytidine), 5′-O-(1-thiophosphate)-guanosine, 5′-O-(1-thiophosphate)-uridine, or 5′-O-(1-thiophosphate)-pseudouridine).

Other internucleoside linkages that may be employed according to the present invention, including internucleoside linkages which do not contain a phosphorous atom, are described herein below.

Combinations of Alternative Sugars, Nucleobases, and Internucleoside Linkages

The polynucleotides of the invention can include a combination of alterations to the sugar, the nucleobase, and/or the internucleoside linkage. These combinations can include any one or more alterations described herein.

### Synthesis of Polynucleotides

The polynucleotide molecules for use in accordance with the invention may be prepared according to any useful technique, as described herein. The alternative nucleosides and nucleotides used in the synthesis of polynucleotide molecules disclosed herein can be prepared from readily available starting materials using the following general methods and procedures. Where typical or preferred process conditions (e.g., reaction temperatures, times, mole ratios of reactants, solvents, pressures, etc.) are provided, a skilled artisan would be able to optimize and develop additional process conditions. Optimum reaction conditions may vary with the particular reactants or solvent used, but such conditions can be determined by one skilled in the art by routine optimization procedures.

The processes described herein can be monitored according to any suitable method known in the art. For example, product formation can be monitored by spectroscopic means, such as nuclear magnetic resonance spectroscopy (e.g., ¹H or ¹³C) infrared spectroscopy, spectrophotometry (e.g., UV-visible), or mass spectrometry, or by chromatography such as high performance liquid chromatography (HPLC) or thin layer chromatography.

Preparation of polynucleotide molecules of the present invention can involve the protection and deprotection of various chemical groups. The need for protection and deprotection, and the selection of appropriate protecting groups can be readily determined by one skilled in the art. The chemistry of protecting groups can be found, for example, in Greene, et al., Protective Groups in Organic Synthesis, 2d. Ed., Wiley & Sons, 1991, which is incorporated herein by reference in its entirety.

The reactions of the processes described herein can be carried out in suitable solvents, which can be readily selected by one of skill in the art of organic synthesis. Suitable solvents can be substantially nonreactive with the starting materials (reactants), the intermediates, or products at the temperatures at which the reactions are carried out, i.e., temperatures which can range from the solvent's freezing temperature to the solvent's boiling temperature. A given reaction can be carried out in one solvent or a mixture of more than one solvent. Depending on the particular reaction step, suitable solvents for a particular reaction step can be selected.

Resolution of racemic mixtures of alternative polynucleotides or nucleic acids (e.g., polynucleotides or mRNA molecules) can be carried out by any of numerous methods known in the art. An example method includes fractional recrystallization using a “chiral resolving acid” which is an optically active, salt-forming organic acid. Suitable resolving agents for fractional recrystallization methods are, for example, optically active acids, such as the D and L forms of tartaric acid, diacetyltartaric acid, dibenzoyltartaric acid, mandelic acid, malic acid, lactic acid or the various optically active camphorsulfonic acids. Resolution of racemic mixtures can also be carried out by elution on a column packed with an optically active resolving agent (e.g., dinitrobenzoylphenylglycine). Suitable elution solvent composition can be determined by one skilled in the art.

Alternative nucleosides and nucleotides (e.g., building block molecules) can be prepared according to the synthetic methods described in Ogata et al., J. Org. Chem. 74:2585-2588 (2009); Purmal et al., Nucl. Acids Res. 22(1): 72-78, (1994); Fukuhara et al., Biochemistry, 1(4): 563-568 (1962); and Xu et al., Tetrahedron, 48(9): 1729-1740 (1992), each of which are incorporated by reference in their entirety.

If the polynucleotide includes one or more alternative nucleosides or nucleotides, the polynucleotides of the invention may or may not be uniformly altered along the entire length of the molecule. For example, one or more or all types of nucleotide (e.g., purine or pyrimidine, or any one or more or all of A, G, U, C) may or may not be uniformly altered in a polynucleotide of the invention, or in a given predetermined sequence region thereof. In some embodiments, all nucleotides X in a polynucleotide of the invention (or in a given sequence region thereof) are altered, wherein X may any one of nucleotides A, G, U, C, or any one of the combinations A+G, A+U, A+C, G+U, G+C, U+C, A+G+U, A+G+C, G+U+C or A+G+C.

Different sugar alterations, nucleotide alterations, and/or internucleoside linkages (e.g., backbone structures) may exist at various positions in the polynucleotide. One of ordinary skill in the art will appreciate that the nucleotide analogs or other alteration(s) may be located at any position(s) of a polynucleotide such that the function of the polynucleotide is not substantially decreased. An alteration may also be a 5′ or 3′ terminal alteration. The polynucleotide may contain from about 1% to about 100% alternative nucleosides, nucleotides, or internucleoside linkages (either in relation to overall nucleotide content, or in relation to one or more types of nucleotide, i.e. any one or more of A, G, U or C) or any intervening percentage (e.g., from 1% to 20%, from 1% to 25%, from 1% to 50%, from 1% to 60%, from 1% to 70%, from 1% to 80%, from 1% to 90%, from 1% to 95%, from 10% to 20%, from 10% to 25%, from 10% to 50%, from 10% to 60%, from 10% to 70%, from 10% to 80%, from 10% to 90%, from 10% to 95%, from 10% to 100%, from 20% to 25%, from 20% to 50%, from 20% to 60%, from 20% to 70%, from 20% to 80%, from 20% to 90%, from 20% to 95%, from 20% to 100%, from 50% to 60%, from 50% to 70%, from 50% to 80%, from 50% to 90%, from 50% to 95%, from 50% to 100%, from 70% to 80%, from 70% to 90%, from 70% to 95%, from 70% to 100%, from 80% to 90%, from 80% to 95%, from 80% to 100%, from 90% to 95%, from 90% to 100%, and from 95% to 100. In some embodiments, the remaining percentage is accounted for by the presence of A, G, U, or C.

When referring to percentage incorporation by an alternative nucleoside, nucleotide, or internucleoside linkage, in some embodiments the remaining percentage necessary to total 100% is accounted for by the corresponding natural nucleoside, nucleotide, or internucleoside linkage. In other embodiments, the remaining percentage necessary to total 100% is accounted for by a second alternative nucleoside, nucleotide, or internucleoside linkage.

### Messenger RNA

The present invention features composition including one or more mRNAs, where each mRNA encodes a polypeptide, Each mRNA includes (i) a 5′-cap structure; (ii) a 5′-UTR; (iii) an open reading frame encoding the polypeptide; (iv) a 3′-untranslated region (3′-UTR); and (v) a poly-A region.

In some embodiments, the mRNA includes from about 30 to about 3,000 nucleotides (e.g., from 30 to 50, from 30 to 100, from 30 to 250, from 30 to 500, from 30 to 750, from 30 to 1,000, from 30 to 1,500, from 30 to 2,000, from 30 to 2,500, from 50 to 100, from 50 to 250, from 50 to 500, from 50 to 750, from 50 to 1,000, from 50 to 1,500, from 50 to 2,000, from 50 to 2,500, from 50 to 3,000, from 100 to 500, from 100 to 750, from 100 to 1,000, from 100 to 1,500, from 100 to 2,000, from 100 to 2,500, from 100 to 3,000, from 500 to 750, from 500 to 1,000, from 500 to 1,500, from 500 to 2,000, from 500 to 2,500, from 500 to 3,000, from 1,000 to 1,500, from 1,000 to 2,000, from 1,000 to 2,500, from 1,000 to 3,000, from 1,500 to 2,000, from 1,500 to 2,500, from 1,500 to 3,000, from 2,000 to 3,000, from 2,000 to 2,500, and from 2,500 to 3,000).

mRNA: 5′-Cap

The 5′-cap structure of an mRNA is involved in nuclear export, increasing mRNA stability and binds the mRNA Cap Binding Protein (CBP), which is responsible for mRNA stability in the cell and translation competency through the association of CBP with poly(A) binding protein to form the mature cyclic mRNA species. The cap further assists the removal of 5′ proximal introns removal during mRNA splicing.

Endogenous mRNA molecules may be 5′-end capped generating a 5′-ppp-5′-triphosphate linkage between a terminal guanosine cap residue and the 5′-terminal transcribed sense nucleotide of the mRNA. This 5′-guanylate cap may then be methylated to generate an N7-methyl-guanylate residue. The ribose sugars of the terminal and/or anteterminal transcribed nucleotides of the 5′ end of the mRNA may optionally also be 2′-O-methylated. 5′-decapping through hydrolysis and cleavage of the guanylate cap structure may target a nucleic acid molecule, such as an mRNA molecule, for degradation.

Alterations to the nucleic acids of the present invention may generate a non-hydrolyzable cap structure preventing decapping and thus increasing mRNA half-life. Because cap structure hydrolysis requires cleavage of 5′-ppp-5′ phosphorodiester linkages, alternative nucleotides may be used during the capping reaction. For example, a Vaccinia Capping Enzyme from New England Biolabs (Ipswich, Mass.) may be used with α-thio-guanosine nucleotides according to the manufacturer's instructions to create a phosphorothioate linkage in the 5′-ppp-5′ cap. Additional alternative guanosine nucleotides may be used such as α-methyl-phosphonate and seleno-phosphate nucleotides.

Additional alterations include, but are not limited to, 2′-O-methylation of the ribose sugars of 5′-terminal and/or 5′-anteterminal nucleotides of the mRNA (as mentioned above) on the 2′-hydroxyl group of the sugar ring. Multiple distinct 5′-cap structures can be used to generate the 5′-cap of a nucleic acid molecule, such as an mRNA molecule.

5′-cap structures include those described in International Patent Publication Nos. WO2008/127688, WO2008/016473, and WO2011/015347, each of which is incorporated herein by reference in its entirety.

Cap analogs, which herein are also referred to as synthetic cap analogs, chemical caps, chemical cap analogs, or structural or functional cap analogs, differ from natural (i.e. endogenous, wild-type or physiological) 5′-caps in their chemical structure, while retaining cap function. Cap analogs may be chemically (i.e. non-enzymatically) or enzymatically synthesized and/linked to a nucleic acid molecule.

For example, the Anti-Reverse Cap Analog (ARCA) cap contains two guanosines linked by a 5′-5′-triphosphate group, wherein one guanosine contains an N7 methyl group as well as a 3′-O-methyl group (i.e., N7,3′-O-dimethyl-guanosine-5′-triphosphate-5′-guanosine (m⁷G-3′mppp-G; which may equivalently be designated 3′ O-Me-m7G(5′)ppp(5′)G). The 3′-O atom of the other, unaltered, guanosine becomes linked to the 5′-terminal nucleotide of the capped nucleic acid molecule (e.g. an mRNA or mmRNA). The N7- and 3′-O-methlyated guanosine provides the terminal moiety of the capped nucleic acid molecule (e.g. mRNA or mmRNA).

Another exemplary cap is mCAP, which is similar to ARCA but has a 2′-O-methyl group on guanosine (i.e., N7,2′-O-dimethyl-guanosine-5′-triphosphate-5′-guanosine, m⁷Gm-ppp-G).

In some embodiments, the cap is a dinucleotide cap analog. As a non-limiting example, the dinucleotide cap analog may be modified at different phosphate positions with a boranophosphate group or a phophoroselenoate group such as the dinucleotide cap analogs described in U.S. Pat. No. 8,519,110, the contents of which are herein incorporated by reference in its entirety.

In some embodiments, the cap analog is a N7-(4-chlorophenoxyethyl) substituted dicnucleotide form of a cap analog known in the art and/or described herein. Non-limiting examples of a N7-(4-chlorophenoxyethyl) substituted dinucleotide form of a cap analog include a N7-(4-chlorophenoxyethyl)-G(5′)ppp(5′)G and a N7-(4-chlorophenoxyethyl)-m^{3-O}G(5′)ppp(5′)G cap analog (See e.g., the various cap analogs and the methods of synthesizing cap analogs described in Kore et al. Bioorganic & Medicinal Chemistry 2013 21:4570-4574; the contents of which are herein incorporated by reference in its entirety). In some embodiments, a cap analog of the present invention is a 4-chloro/bromophenoxyethyl analog.

While cap analogs allow for the concomitant capping of a nucleic acid molecule in an in vitro transcription reaction, up to 20% of transcripts remain uncapped. This, as well as the structural differences of a cap analog from endogenous 5′-cap structures of nucleic acids produced by the endogenous, cellular transcription machinery, may lead to reduced translational competency and reduced cellular stability.

Nucleic acids of the invention (e.g., mRNAs of the invention) may also be capped post-transcriptionally, using enzymes. 5′ cap structures produced by enzymatic capping may enhance binding of cap binding proteins, increase half-life, reduce susceptibility to 5′ endonucleases and/or reduce 5′ decapping, as compared to synthetic 5′-cap structures known in the art (or to a wild-type, natural or physiological 5′-cap structure). For example, recombinant Vaccinia Virus Capping Enzyme and recombinant 2′-O-methyltransferase enzyme can create a canonical 5′-5′-triphosphate linkage between the 5′-terminal nucleotide of an mRNA and a guanosine cap nucleotide wherein the cap guanosine contains an N7 methylation and the 5′-terminal nucleotide of the mRNA contains a 2′-O-methyl. Such a structure is termed the Cap1 structure. This cap results in a higher translational-competency and cellular stability and a reduced activation of cellular pro-inflammatory cytokines, as compared, e.g., to other 5′cap analog structures known in the art. Cap structures include 7mG(5′)ppp(5′)N, pN2p (cap 0), 7mG(5′)ppp(5′)NImpNp (cap 1), 7mG(5′)-ppp(5′)NImpN2mp (cap 2) and m(7)Gpppm(3)(6,6,2′)Apm(2′)Apm(2′)Cpm(2)(3,2′)Up (cap 4).

According to the present invention, 5′ terminal caps may include endogenous caps or cap analogs. According to the present invention, a 5′ terminal cap may include a guanosine analog. Useful guanosine analogs include inosine, N1-methyl-guanosine, 2′-fluoro-guanosine, 7-deaza-guanosine, 8-oxo-guanosine, 2-amino-guanosine, LNA-guanosine, and 2-azido-guanosine.

In some embodiments, the nucleic acids described herein may contain a modified 5′-cap. A modification on the 5′-cap may increase the stability of mRNA, increase the half-life of the mRNA, and could increase the mRNA translational efficiency. The modified 5′-cap may include, but is not limited to, one or more of the following modifications: modification at the 2′ and/or 3′ position of a capped guanosine triphosphate (GTP), a replacement of the sugar ring oxygen (that produced the carbocyclic ring) with a methylene moiety (CH₂), a modification at the triphosphate bridge moiety of the cap structure, or a modification at the nucleobase (G) moiety.

mRNA: Coding Region

Provided are nucleic acids that encode polypeptides. Polypeptides encoded by mRNA of the invention may correspond to known proteins. Polypeptides of the invention have a certain identity with a reference polypeptide sequence (e.g., a known protein, such a protein associated with a disease or condition). The term “identity” refers to a relationship between the sequences of two or more peptides, as determined by comparing the sequences. Identity described the degree of sequence relatedness between peptides, as determined by the number of matches between strings of two or more amino acid residues. Identity of related peptides can be readily calculated by known methods. Such methods include, but are not limited to, those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987; Sequence Analysis Primer, Gribskov, M. and Devereux, J., eds., M. Stockton Press, New York, 1991; and Carillo et al., SIAM J. Applied Math. 48, 1073 (1988).

In some embodiments, the polypeptide variant has the same or a similar activity as a reference polypeptide. Alternatively, the polypeptide encoded by the mRNA is a variant of a reference polypeptide. The variant polypeptide may have altered activity (e.g., increased or decreased biological activity) relative to a reference polypeptide. Generally, variants of a particular polynucleotide or polypeptide of the present disclosure will have at least about 40%, 45%, 50%, 55%, 60%, 65%, 70%, 75%, 80%, 85%, 90%, 91%, 92%, 93%, 94%, 95%, 96%, 97%, 98%, 99% or more sequence identity to that particular reference polynucleotide or polypeptide as determined by sequence alignment programs and parameters described herein and known to those skilled in the art.

As recognized by those skilled in the art, protein fragments, functional protein domains, and homologous proteins are also considered to be within the scope of this present disclosure. For example, provided herein is any protein fragment of a reference protein (meaning a polypeptide sequence at least one amino acid residue shorter than a reference polypeptide sequence but otherwise identical) 10, 20, 30, 40, 50, 60, 70, 80, 90, 100 or greater than 100 amino acids in length In another example, any protein that includes a stretch of about 20, about 30, about 40, about 50, or about 100 amino acids which are about 40%, about 50%, about 60%, about 70%, about 80%, about 90%, about 95%, or about 100% identical to any of the sequences described herein can be utilized in accordance with the present disclosure. In certain embodiments, a protein sequence to be utilized in accordance with the present disclosure includes 2, 3, 4, 5, 6, 7, 8, 9, 10, or more mutations as shown in any of the sequences provided or referenced herein.

Erythropoietin (EPO) and granulocyte colony-stimulating factor (GCSF) are exemplary polypeptides of interest.

mRNA: Poly-A Tail

During RNA processing, a long chain of adenosine nucleotides (poly-A tail) is normally added to a messenger RNA (mRNA) molecules to increase the stability of the molecule. Immediately after transcription, the 3′ end of the transcript is cleaved to free a 3′ hydroxyl. Then poly-A polymerase adds a chain of adenosine nucleotides to the RNA. The process, called polyadenylation, adds a poly-A tail that is between 100 and 250 residues long.

Methods for the stabilization of RNA by incorporation of chain-terminating nucleosides at the 3′-terminus include those described in International Patent Publication No. WO2013/103659, incorporated herein in its entirety.

Poly(A) tail deadenylation by 3′ exonucleases is a key step in cellular mRNA degradation in eukaryotes. By blocking 3′ exonucleases, the functional half-life of mRNA can be increased, resulting in increase protein expression. Chemical and enzymatic ligation strategies to modify the 3′ end of mRNA with reverse chirality adenosine (LA10) and/or inverted deoxythymidine (IdT) are known to those of skill in the art, and have been demonstrated to extend mRNA half-life in cellular and in vivo studies. In some embodiments, the poly(A)tail of the mRNA includes a 3′ LA10 or IdT modification. For example, as described in International Patent Publication No. WO2017/049275, the tail modifications of which are incorporated by reference in their entirety.

Additional strategies have been explored to further stabilize mRNA, including: chemical modification of the 3′ nucleotide (e.g., conjugation of a morpholino to the 3′ end of the poly(A)tail); incorporation of stabilizing sequences after the poly(A) tail (e.g., a co-polymer, a stem-loop, or a triple helix); and/or annealing of structured oligos to the 3′ end of an mRNA, as described, for example, in International Patent Publication No. WO2017/049286, the stabilized linkages of which are incorporated by reference in their entirety.

Annealing an oligonucleotide (e.g., an oligonucleotide conjugate) with a complex secondary structure (e.g., a triple-helix structure or a stem-loop structure) at the 3′end may provide nuclease resistance and increase half-life of mRNA.

Unique poly-A tail lengths may provide certain advantages to the RNAs of the present invention. Generally, the length of a poly-A tail of the present invention is greater than 30 nucleotides in length. In some embodiments, the poly-A tail is greater than 35 nucleotides in length. In some embodiments, the length is at least 40 nucleotides. n another embodiment, the length is at least 45 nucleotides. In some embodiments, the length is at least 55 nucleotides. In some embodiments, the length is at least 60 nucleotides. In another embodiment, the length is at least 60 nucleotides. In some embodiments, the length is at least 80 nucleotides. In some embodiments, the length is at least 90 nucleotides. In some embodiments, the length is at least 100 nucleotides. In some embodiments, the length is at least 120 nucleotides. In some embodiments, the length is at least 140 nucleotides. In some embodiments, the length is at least 160 nucleotides. In some embodiments, the length is at least 180 nucleotides. In some embodiments, the length is at least 200 nucleotides. In some embodiments, the length is at least 250 nucleotides. In some embodiments, the length is at least 300 nucleotides. In some embodiments, the length is at least 350 nucleotides. In some embodiments, the length is at least 400 nucleotides. In some embodiments, the length is at least 450 nucleotides. In some embodiments, the length is at least 500 nucleotides. In some embodiments, the length is at least 600 nucleotides. In some embodiments, the length is at least 700 nucleotides. In some embodiments, the length is at least 800 nucleotides. In some embodiments, the length is at least 900 nucleotides. In some embodiments, the length is at least 1000 nucleotides. In some embodiments, the length is at least 1100 nucleotides. In some embodiments, the length is at least 1200 nucleotides. In some embodiments, the length is at least 1300 nucleotides. In some embodiments, the length is at least 1400 nucleotides. In some embodiments, the length is at least 1500 nucleotides. In some embodiments, the length is at least 1600 nucleotides. In some embodiments, the length is at least 1700 nucleotides. In some embodiments, the length is at least 1800 nucleotides. In some embodiments, the length is at least 1900 nucleotides. In some embodiments, the length is at least 2000 nucleotides. In some embodiments, the length is at least 2500 nucleotides. In some embodiments, the length is at least 3000 nucleotides.

In some embodiments, the poly-A tail may be 80 nucleotides, 120 nucleotides, 160 nucleotides in length. In some embodiments, the poly-A tail may be 20, 40, 80, 100, 120, 140 or 160 nucleotides in length.

In some embodiments, the poly-A tail is designed relative to the length of the mRNA. This design may be based on the length of the coding region of the mRNA, the length of a particular feature or region of the mRNA, or based on the length of the ultimate product expressed from the RNA. When relative to any additional feature of the RNA (e.g., other than the mRNA portion which includes the poly-A tail) the poly-A tail may be 10, 20, 30, 40, 50, 60, 70, 80, 90 or 100% greater in length than the additional feature. The poly-A tail may also be designed as a fraction of the mRNA to which it belongs. In this context, the poly-A tail may be 10, 20, 30, 40, 50, 60, 70, 80, or 90% or more of the total length of the construct or the total length of the construct minus the poly-A tail.

In some embodiments, engineered binding sites and/or the conjugation of nucleic acids or mRNA for Poly-A binding protein may be used to enhance expression. The engineered binding sites may be sensor sequences which can operate as binding sites for ligands of the local microenvironment of the nucleic acids and/or mRNA. As a non-limiting example, the nucleic acids and/or mRNA may include at least one engineered binding site to alter the binding affinity of Poly-A binding protein (PABP) and analogs thereof. The incorporation of at least one engineered binding site may increase the binding affinity of the PABP and analogs thereof.

Additionally, multiple distinct nucleic acids or mRNA may be linked together to the PABP (Poly-A binding protein) through the 3′-end using nucleotides at the 3′-terminus of the poly-A tail. Transfection experiments can be conducted in relevant cell lines at and protein production can be assayed by ELISA at 12 hr, 24 hr, 48 hr, 72 hr and day 7 post-transfection. As a non-limiting example, the transfection experiments may be used to evaluate the effect on PABP or analogs thereof binding affinity as a result of the addition of at least one engineered binding site.

In some embodiments, a polyA tail may be used to modulate translation initiation. While not wishing to be bound by theory, the polyA tail recruits PABP which in turn can interact with translation initiation complex and thus may be essential for protein synthesis.

In some embodiments, a polyA tail may also be used in the present invention to protect against 3′-5′ exonuclease digestion.

In some embodiments, the nucleic acids or mRNA of the present invention are designed to include a polyA-G Quartet. The G-quartet is a cyclic hydrogen bonded array of four guanosine nucleotides that can be formed by G-rich sequences in both DNA and RNA. In this embodiment, the G-quartet is incorporated at the end of the poly-A tail. The resultant nucleic acid or mRNA may be assayed for stability, protein production and other parameters including half-life at various time points. It has been discovered that the polyA-G quartet results in protein production equivalent to at least 75% of that seen using a poly-A tail of 120 nucleotides alone.

In some embodiments, the nucleic acids or mRNA of the present invention may include a polyA tail and may be stabilized by the addition of a chain terminating nucleoside. The nucleic acids and/or mRNA with a polyA tail may further include a 5′cap structure.

In some embodiments, the nucleic acids or mRNA of the present invention may include a polyA-G Quartet. The nucleic acids and/or mRNA with a polyA-G Quartet may further include a 5′cap structure.

In some embodiments, the chain terminating nucleoside which may be used to stabilize the nucleic acid or mRNA including a polyA tail or polyA-G Quartet may be, but is not limited to, those described in International Patent Publication No. WO2013103659, incorporated herein by reference in its entirety. In some embodiments, the chain terminating nucleosides which may be used with the present invention includes, but is not limited to, 3′-deoxyadenosine (cordycepin), 3′-deoxyuridine, 3′-deoxycytosine, 3′-deoxyguanosine, 3′-deoxythymine, 2′,3′-dideoxynucleosides, such as 2′,3′-dideoxyadenosine, 2′,3′-dideoxyuridine, 2′,3′-dideoxycytosine, 2′,3′-dideoxyguanosine, 2′,3′-dideoxythymine, a 2′-deoxynucleoside, or a —O— methylnucleoside.

In some embodiments, the mRNA which includes a polyA tail or a polyA-G Quartet may be stabilized by an alteration to the 3′region of the nucleic acid that can prevent and/or inhibit the addition of oligio(U) (see e.g., International Patent Publication No. WO2013/103659, incorporated herein by reference in its entirety).

In yet another embodiment, the mRNA, which includes a polyA tail or a polyA-G Quartet may be stabilized by the addition of an oligonucleotide that terminates in a 3′-deoxynucleoside, 2′,3′-dideoxynucleoside 3′-O-methylnucleosides, 3′-O-ethylnucleosides, 3′-arabinosides, and other alternative nucleosides known in the art and/or described herein.

mRNA: Stem-Loops

In some embodiments, the nucleic acids of the present invention (e.g., the mRNA of the present invention) may include a stem-loop such as, but not limited to, a histone stem-loop. The stem-loop may be a nucleotide sequence that is about 25 or about 26 nucleotides in length such as, but not limited to, SEQ ID NOs: 7-17 as described in International Patent Publication No. WO2013/103659, incorporated herein by reference in its entirety. The histone stem-loop may be located 3′ relative to the coding region (e.g., at the 3′ terminus of the coding region). As a non-limiting example, the stem-loop may be located at the 3′ end of a nucleic acid described herein.

In some embodiments, the stem-loop may be located in the second terminal region. As a non-limiting example, the stem-loop may be located within an untranslated region (e.g., 3′-UTR) in the second terminal region.

In some embodiments, the nucleic acid such as, but not limited to mRNA, which includes the histone stem-loop may be stabilized by the addition of at least one chain terminating nucleoside. Not wishing to be bound by theory, the addition of at least one chain terminating nucleoside may slow the degradation of a nucleic acid and thus can increase the half-life of the nucleic acid.

In some embodiments, the chain terminating nucleoside may be, but is not limited to, those described in International Patent Publication No. WO2013/103659, incorporated herein by reference in its entirety. In some embodiments, the chain terminating nucleosides which may be used with the present invention includes, but is not limited to, 3′-deoxyadenosine (cordycepin), 3′-deoxyuridine, 3′-deoxycytosine, 3′-deoxyguanosine, 3′-deoxythymine, 2′,3′-dideoxynucleosides, such as 2′,3′-dideoxyadenosine, 2′,3′-dideoxyuridine, 2′,3′-dideoxycytosine, 2′,3′-dideoxyguanosine, 2′,3′-dideoxythymine, a 2′-deoxynucleoside, or a —O— methylnucleoside.

In some embodiments, the nucleic acid such as, but not limited to mRNA, which includes the histone stem-loop may be stabilized by an alteration to the 3′region of the nucleic acid that can prevent and/or inhibit the addition of oligio(U) (see e.g., International Patent Publication No. WO2013/103659, incorporated herein by reference in its entirety).

In yet another embodiment, the nucleic acid such as, but not limited to mRNA, which includes the histone stem-loop may be stabilized by the addition of an oligonucleotide that terminates in a 3′-deoxynucleoside, 2′,3′-dideoxynucleoside 3′-O-methylnucleosides, 3′-O-ethylnucleosides, 3′-arabinosides, and other alternative nucleosides known in the art and/or described herein.

In some embodiments, the nucleic acids of the present invention may include a histone stem-loop, a polyA tail sequence and/or a 5′cap structure. The histone stem-loop may be before and/or after the polyA tail sequence. The nucleic acids including the histone stem-loop and a polyA tail sequence may include a chain terminating nucleoside described herein.

In some embodiments, the nucleic acids of the present invention may include a histone stem-loop and a 5′cap structure. The 5′-cap structure may include, but is not limited to, those described herein and/or known in the art.

In some embodiments, the conserved stem-loop region may include a miR sequence described herein. As a non-limiting example, the stem-loop region may include the seed sequence of a miR sequence described herein. In another non-limiting example, the stem-loop region may include a miR-122 seed sequence.

In some embodiments, the conserved stem-loop region may include a miR sequence described herein and may also include a TEE sequence.

In some embodiments, the incorporation of a miR sequence and/or a TEE sequence changes the shape of the stem-loop region which may increase and/or decrease translation. (see e.g., Kedde et al. A Pumilio-induced RNA structure switch in p27-3′-UTR controls miR-221 and miR-22 accessibility. Nature Cell Biology. 2010, herein incorporated by reference in its entirety).

In some embodiments, the nucleic acids described herein may include at least one histone stem-loop and a polyA sequence or polyadenylation signal. Non-limiting examples of nucleic acid sequences encoding for at least one histone stem-loop and a polyA sequence or a polyadenylation signal are described in International Patent Publication Nos. WO2013/120497, WO2013/120629, WO2013/120500, WO2013/120627, WO2013/120498, WO2013/120626, WO2013/120499 and WO2013/120628, the contents of each of which are incorporated herein by reference in their entirety. In some embodiments, the nucleic acid encoding for a histone stem-loop and a polyA sequence or a polyadenylation signal may code for a pathogen antigen or fragment thereof such as the nucleic acid sequences described in International Patent Publication Nos. WO2013/120499 and WO2013/120628, the contents of both of which are incorporated herein by reference in their entirety. In some embodiments, the nucleic acid encoding for a histone stem-loop and a polyA sequence or a polyadenylation signal may code for a therapeutic protein such as the nucleic acid sequences described in International Patent Publication Nos. WO2013/120497 and WO2013/120629, the contents of both of which are incorporated herein by reference in their entirety. In some embodiments, the nucleic acid encoding for a histone stem-loop and a polyA sequence or a polyadenylation signal may code for a tumor antigen or fragment thereof such as the nucleic acid sequences described in International Patent Publication Nos. WO2013/120500 and WO2013/120627, the contents of both of which are incorporated herein by reference in their entirety. In some embodiments, the nucleic acid encoding for a histone stem-loop and a polyA sequence or a polyadenylation signal may code for an allergenic antigen or an autoimmune self-antigen such as the nucleic acid sequences described in International Patent Publication Nos. WO2013/120498 and WO2013/120626, the contents of both of which are incorporated herein by reference in their entirety.

mRNA: Triple Helices

In some embodiments, nucleic acids of the present invention (e.g., the mRNA of the present invention) may include a triple helix on the 3′ end of the nucleic acid. The 3′ end of the nucleic acids of the present invention may include a triple helix alone or in combination with a Poly-A tail.

In some embodiments, the nucleic acid of the present invention may include at least a first and a second U-rich region, a conserved stem-loop region between the first and second region and an A-rich region. The first and second U-rich region and the A-rich region may associate to form a triple helix on the 3′ end of the nucleic acid. This triple helix may stabilize the nucleic acid, enhance the translational efficiency of the nucleic acid and/or protect the 3′ end from degradation. Triple helices include, but are not limited to, the triple helix sequence of metastasis-associated lung adenocarcinoma transcript 1 (MALAT1), MEN-β and polyadenylated nuclear (PAN) RNA (See Wilusz et al., Genes & Development 2012 26:2392-2407; herein incorporated by reference in its entirety).

In some embodiments, the triple helix may be formed from the cleavage of a MALAT1 sequence prior to the cloverleaf structure. While not meaning to be bound by theory, MALAT1 is a long non-coding RNA which, when cleaved, forms a triple helix and a tRNA-like cloverleaf structure. The MALAT1 transcript then localizes to nuclear speckles and the tRNA-like cloverleaf localizes to the cytoplasm (Wilusz et al. Cell 2008 135(5): 919-932; incorporated herein by reference in its entirety).

As a non-limiting example, the terminal end of the nucleic acid of the present invention including the MALAT1 sequence can then form a triple helix structure, after RNaseP cleavage from the cloverleaf structure, which stabilizes the nucleic acid (Peart et al. Non-mRNA 3′ end formation: how the other half lives; WIREs RNA 2013; incorporated herein by reference in its entirety).

In some embodiments, the nucleic acids or mRNA described herein include a MALAT1 sequence. In some embodiments, the nucleic acids or mRNA may be polyadenylated. In yet another embodiment, the nucleic acids or mRNA is not polyadenylated but has an increased resistance to degradation compared to unaltered nucleic acids or mRNA.

In some embodiments, the nucleic acids of the present invention may include a MALAT1 sequence in the second flanking region (e.g., the 3′-UTR). As a non-limiting example, the MALAT1 sequence may be human or mouse.

In some embodiments, the cloverleaf structure of the MALAT1 sequence may also undergo processing by RNaseZ and CCA adding enzyme to form a tRNA-like structure called mascRNA (MALAT1-associated small cytoplasmic RNA). As a non-limiting example, the mascRNA may encode a protein or a fragment thereof and/or may include a microRNA sequence. The mascRNA may include at least one chemical alteration described herein.

mRNA: Translation Enhancer Elements (TEEs)

The term “translational enhancer element” or “translation enhancer element” (herein collectively referred to as “TEE”) refers to sequences that increase the amount of polypeptide or protein produced from an mRNA. TEEs are conserved elements in the UTR which can promote translational activity of a nucleic acid such as, but not limited to, cap-dependent or cap-independent translation. The conservation of these sequences has been previously shown by Panek et al (Nucleic Acids Research, 2013, 1-10; incorporated herein by reference in its entirety) across 14 species including humans.

In some embodiments, the 5′-UTR of the mRNA includes at least one TEE. The TEE may be located between the transcription promoter and the start codon. The mRNA with at least one TEE in the 5′-UTR may include a cap at the 5′-UTR. Further, at least one TEE may be located in the 5′-UTR of mRNA undergoing cap-dependent or cap-independent translation.

The TEEs known may be in the 5′-leader of the Gtx homeodomain protein (Chappell et al., Proc. Natl. Acad. Sci. USA 101:9590-9594, 2004, incorporated herein by reference in their entirety).

In another non-limiting example, TEEs are disclosed as SEQ ID NOs: 1-35 in US Patent Publication No. US20090226470, SEQ ID NOs: 1-35 in US Patent Publication No. US20130177581, SEQ ID NOs: 1-35 in International Patent Publication No. WO2009075886, SEQ ID NOs: 1-5, and 7-645 in International Patent Publication No. WO2012009644, SEQ ID NO: 1 in International Patent Publication No. WO1999024595, SEQ ID NO: 1 in U.S. Pat. No. 6,310,197, and SEQ ID NO: 1 in U.S. Pat. No. 6,849,405, each of which is incorporated herein by reference in its entirety.

The TEE may be an internal ribosome entry site (IRES), HCV-IRES or an IRES element such as, but not limited to, those described in U.S. Pat. No. 7,468,275, US Patent Publication Nos. US20070048776 and US20110124100 and International Patent Publication Nos. WO2007025008 and WO2001055369, each of which is incorporated herein by reference in its entirety. The IRES elements may include, but are not limited to, the Gtx sequences (e.g., Gtx9-nt, Gtx8-nt, Gtx7-nt) described by Chappell et al. (Proc. Natl. Acad. Sci. USA 101:9590-9594, 2004) and Zhou et al. (PNAS 102:6273-6278, 2005) and in US Patent Publication Nos. US20070048776 and US20110124100 and International Patent Publication No. WO2007025008, each of which is incorporated herein by reference in its entirety.

Additional exemplary TEEs are disclosed in U.S. Pat. Nos. 6,310,197, 6,849,405, 7,456,273, 7,183,395; US Patent Publication Nos. US20090226470, US20070048776, US20110124100, US20090093049, US20130177581; International Patent Publication Nos. WO2009075886, WO2007025008, WO2012009644, WO2001055371 WO1999024595; and European Patent Publications Nos. EP2610341A1 and EP2610340A1; each of which is incorporated herein by reference in its entirety.

In some embodiments, the polynucleotides, primary constructs, alternative nucleic acids and/or mRNA may include at least one TEE that is described in International Patent Publication Nos. WO1999024595, WO2012009644, WO2009075886, WO2007025008, WO1999024595, European Patent Publication Nos. EP2610341A1 and EP2610340A1, U.S. Pat. Nos. 6,310,197, 6,849,405, 7,456,273, 7,183,395, US Patent Publication No. US20090226470, US20110124100, US20070048776, US20090093049, and US20130177581 each of which is incorporated herein by reference in its entirety. The TEE may be located in the 5′-UTR of the mRNA.

In some embodiments, the polynucleotides, primary constructs, alternative nucleic acids and/or mmRNA may include at least one TEE that has at least 50%, at least 55%, at least 60%, at least 65%, at least 70%, at least 75%, at least 80%, at least 85%, at least 90%, at least 95% or at least 99% identity with the TEEs described in US Patent Publication Nos. US20090226470, US20070048776, US20130177581 and US20110124100, International Patent Publication Nos. WO1999024595, WO2012009644, WO2009075886 and WO2007025008, European Patent Publication No. EP2610341A1 and EP2610340A1, and U.S. Pat. Nos. 6,310,197, 6,849,405, 7,456,273, 7,183,395, each of which is incorporated herein by reference in its entirety.

Multiple copies of a specific TEE can be present in mRNA. The TEEs in the translational enhancer polynucleotides can be organized in one or more sequence segments. A sequence segment can harbor one or more of the specific TEEs exemplified herein, with each TEE being present in one or more copies. When multiple sequence segments are present in a translational enhancer polynucleotide, they can be homogenous or heterogeneous. Thus, the multiple sequence segments in a translational enhancer polynucleotide can harbor identical or different types of the specific TEEs exemplified herein, identical or different number of copies of each of the specific TEEs, and/or identical or different organization of the TEEs within each sequence segment.

In some embodiments, the 5′-UTR of the mRNA may include at least 1, at least 2, at least 3, at least 4, at least 5, at least 6, at least 7, at least 8, at least 9, at least 10, at least 11, at least 12, at least 13, at least 14, at least 15, at least 16, at least 17, at least 18 at least 19, at least 20, at least 21, at least 22, at least 23, at least 24, at least 25, at least 30, at least 35, at least 40, at least 45, at least 50, at least 55 or more than 60 TEE sequences. The TEE sequences in the 5′-UTR of mRNA of the present invention may be the same or different TEE sequences. The TEE sequences may be in a pattern such as ABABAB or AABBAABBAABB or ABCABCABC or variants thereof repeated once, twice, or more than three times. In these patterns, each letter, A, B, or C represent a different TEE sequence at the nucleotide level.

In some embodiments, the 5′-UTR may include a spacer to separate two TEE sequences. As a non-limiting example, the spacer may be a 15 nucleotide spacer and/or other spacers known in the art. As another non-limiting example, the 5′-UTR may include a TEE sequence-spacer module repeated at least once, at least twice, at least 3 times, at least 4 times, at least 5 times, at least 6 times, at least 7 times, at least 8 times and at least 9 times or more than 9 times in the 5′-UTR.

In some embodiments, the spacer separating two TEE sequences may include other sequences known in the art which may regulate the translation of the mRNA of the present invention such as, but not limited to, miR sequences described herein (e.g., miR binding sites and miR seeds). As a non-limiting example, each spacer used to separate two TEE sequences may include a different miR sequence or component of a miR sequence (e.g., miR seed sequence).

In some embodiments, the TEE in the 5′-UTR of the mRNA of the present invention may include at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99% or more than 99% of the TEE sequences disclosed in US Patent Publication Nos. US20090226470, US20070048776, US20130177581 and US20110124100, International Patent Publication Nos. WO1999024595, WO2012009644, WO2009075886 and WO2007025008, European Patent Publication Nos. EP2610341A1 and EP2610340A1, and U.S. Pat. Nos. 6,310,197, 6,849,405, 7,456,273, and 7,183,395 each of which is incorporated herein by reference in its entirety. In some embodiments, the TEE in the 5′-UTR of the mRNA of the present invention may include a 5-30 nucleotide fragment, a 5-25 nucleotide fragment, a 5-20 nucleotide fragment, a 5-15 nucleotide fragment, a 5-10 nucleotide fragment of the TEE sequences disclosed in US Patent Publication Nos. US20090226470, US20070048776, US20130177581, and US20110124100, International Patent Publication No. WO1999024595, WO2012009644, WO2009075886, and WO2007025008, European Patent Publication No. EP2610341A1 and EP2610340A1, and U.S. Pat. Nos. 6,310,197, 6,849,405, 7,456,273, and 7,183,395; each of which is incorporated herein by reference in its entirety.

In some embodiments, the TEE in the 5′-UTR of the mRNA of the present invention may include at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99% or more than 99% of the TEE sequences disclosed in Chappell et al. (Proc. Natl. Acad. Sci. USA 101:9590-9594, 2004) and Zhou et al. (PNAS 102:6273-6278, 2005), in Supplemental Table 1 and in Supplemental Table 2 disclosed by Wellensiek et al (Genome-wide profiling of human cap-independent translation-enhancing elements, Nature Methods, 2013; DOI:10.1038/NMETH.2522); each of which is herein incorporated by reference in its entirety. In some embodiments, the TEE in the 5′-UTR of the polynucleotides, primary constructs, alternative nucleic acids and/or mmRNA of the present invention may include a 5-30 nucleotide fragment, a 5-25 nucleotide fragment, a 5-20 nucleotide fragment, a 5-15 nucleotide fragment, a 5-10 nucleotide fragment of the TEE sequences disclosed in Chappell et al. (Proc. Natl. Acad. Sci. USA 101:9590-9594, 2004) and Zhou et al. (PNAS 102:6273-6278, 2005), in Supplemental Table 1 and in Supplemental Table 2 disclosed by Wellensiek et al (Genome-wide profiling of human cap-independent translation-enhancing elements, Nature Methods, 2013; DOI:10.1038/NMETH.2522); each of which is incorporated herein by reference in its entirety.

In some embodiments, the TEE used in the 5′-UTR of the mRNA of the present invention is an IRES sequence such as, but not limited to, those described in U.S. Pat. No. 7,468,275 and International Patent Publication No. WO2001055369, each of which is incorporated herein by reference in its entirety.

In some embodiments, the TEEs used in the 5′-UTR of the mRNA of the present invention may be identified by the methods described in US Patent Publication Nos. US20070048776 and US20110124100 and International Patent Publication Nos. WO2007025008 and WO2012009644, each of which is incorporated herein by reference in its entirety.

In some embodiments, the TEEs used in the 5′-UTR of the mRNA of the present invention may be a transcription regulatory element described in U.S. Pat. Nos. 7,456,273 and 7,183,395, US Patent Publication No. US20090093049, and International Publication No. WO2001055371, each of which is incorporated herein by reference in its entirety. The transcription regulatory elements may be identified by methods known in the art, such as, but not limited to, the methods described in U.S. Pat. Nos. 7,456,273 and 7,183,395, US Patent Publication No. US20090093049, and International Publication No. WO2001055371, each of which is incorporated herein by reference in its entirety.

In yet another embodiment, the TEE used in the 5′-UTR of the mRNA of the present invention is an oligonucleotide or portion thereof as described in U.S. Pat. Nos. 7,456,273 and 7,183,395, US Patent Publication No. US20090093049, and International Publication No. WO2001055371, each of which is incorporated herein by reference in its entirety.

The 5′-UTR including at least one TEE described herein may be incorporated in a monocistronic sequence such as, but not limited to, a vector system or a nucleic acid vector. As a non-limiting example, the vector systems and nucleic acid vectors may include those described in U.S. Pat. Nos. 7,456,273 and 7,183,395, US Patent Publication Nos. US20070048776, US20090093049, and US20110124100 and International Patent Publication Nos. WO2007025008 and WO2001055371, each of which is incorporated herein by reference in its entirety.

In some embodiments, the TEEs described herein may be located in the 5′-UTR and/or the 3′-UTR of the mRNA. The TEEs located in the 3′-UTR may be the same and/or different than the TEEs located in and/or described for incorporation in the 5′-UTR.

In some embodiments, the 3′-UTR of the mRNA may include at least 1, at least 2, at least 3, at least 4, at least 5, at least 6, at least 7, at least 8, at least 9, at least 10, at least 11, at least 12, at least 13, at least 14, at least 15, at least 16, at least 17, at least 18 at least 19, at least 20, at least 21, at least 22, at least 23, at least 24, at least 25, at least 30, at least 35, at least 40, at least 45, at least 50, at least 55 or more than 60 TEE sequences. The TEE sequences in the 3′-UTR of the polynucleotides, primary constructs, alternative nucleic acids and/or mmRNA of the present invention may be the same or different TEE sequences. The TEE sequences may be in a pattern such as ABABAB or AABBAABBAABB or ABCABCABC or variants thereof repeated once, twice, or more than three times. In these patterns, each letter, A, B, or C represent a different TEE sequence at the nucleotide level.

In some embodiments, the 3′-UTR may include a spacer to separate two TEE sequences. As a non-limiting example, the spacer may be a 15-nucleotide spacer and/or other spacers known in the art. As another non-limiting example, the 3′-UTR may include a TEE sequence-spacer module repeated at least once, at least twice, at least 3 times, at least 4 times, at least 5 times, at least 6 times, at least 7 times, at least 8 times and at least 9 times or more than 9 times in the 3′-UTR.

In some embodiments, the spacer separating two TEE sequences may include other sequences known in the art which may regulate the translation of the mRNA of the present invention such as, but not limited to, miR sequences described herein (e.g., miR binding sites and miR seeds). As a non-limiting example, each spacer used to separate two TEE sequences may include a different miR sequence or component of a miR sequence (e.g., miR seed sequence).

In some embodiments, the incorporation of a miR sequence and/or a TEE sequence changes the shape of the stem-loop region which may increase and/or decrease translation. (see e.g., Kedde et al. A Pumilio-induced RNA structure switch in p27-3′-UTR controls miR-221 and miR-22 accessibility. Nature Cell Biology. 2010, herein incorporated by reference in its entirety).

mRNA: Heterologous 5′-UTRs

5′-UTRs of an mRNA of the invention may be homologous or heterologous to the coding region found in the mRNA. Multiple 5′ UTRs may be included in mRNA and may be the same or of different sequences. Any portion of the mRNA, including none, may be codon optimized and any may independently contain one or more different structural or chemical alterations, before and/or after codon optimization.

Shown in Lengthy Table 21 in International Patent Publication No. WO 2014/081507, and in Lengthy Table 21 and in Table 22 in International Patent Publication No. WO 2014/081507, the contents of each of which are incorporated herein by reference in their entirety, is a listing of the start and stop site of mRNAs. In Table 21 each 5′-UTR (5′-UTR-005 to 5′-UTR 68511) is identified by its start and stop site relative to its native or wild type (homologous) transcript (ENST; the identifier used in the ENSEMBL database).

To alter one or more properties of the mRNA of the invention, 5′-UTRs which are heterologous to the coding region of the mRNA are engineered into the mRNA. The mRNA (e.g., an mRNA in a composition described herein) is administered to cells, tissue or organisms and outcomes such as protein level, localization and/or half-life are measured to evaluate the beneficial effects the heterologous 5′-UTR may have on mRNA. Variants of the 5′ UTRs may be utilized wherein one or more nucleotides are added or removed to the termini, including A, T, C or G. 5′-UTRs may also be codon-optimized or altered in any manner described herein.

mRNA: RNA Motifs for RNA Binding Proteins

RNA binding proteins (RBPs) can regulate numerous aspects of co- and post-transcription gene expression such as, but not limited to, RNA splicing, localization, translation, turnover, polyadenylation, capping, alteration, export and localization. RNA-binding domains (RBDs), such as, but not limited to, RNA recognition motif (RR) and hnRNP K-homology (KH) domains, typically regulate the sequence association between RBPs and their RNA targets (Ray et al. Nature 2013. 499:172-177; incorporated herein by reference in its entirety). In some embodiments, the canonical RBDs can bind short RNA sequences. In some embodiments, the canonical RBDs can recognize structure RNAs.

In some embodiments, to increase the stability of the mRNA of interest, an mRNA encoding HuR is co-transfected or co-injected along with the mRNA of interest into the cells or into the tissue. These proteins can also be tethered to the mRNA of interest in vitro and then administered to the cells together. Poly A tail binding protein, PABP interacts with eukaryotic translation initiation factor eIF4G to stimulate translational initiation. Co-administration of mRNAs encoding these RBPs along with the mRNA drug and/or tethering these proteins to the mRNA drug in vitro and administering the protein-bound mRNA into the cells can increase the translational efficiency of the mRNA. The same concept can be extended to co-administration of mRNA along with mRNAs encoding various translation factors and facilitators as well as with the proteins themselves to influence RNA stability and/or translational efficiency.

In some embodiments, the nucleic acids and/or mRNA may include at least one RNA-binding motif such as, but not limited to a RNA-binding domain (RBD).

In some embodiments, the RBD may be any of the RBDs, fragments or variants thereof descried by Ray et al. (Nature 2013. 499:172-177; incorporated herein by reference in its entirety).

In some embodiments, the nucleic acids or mRNA of the present invention may include a sequence for at least one RNA-binding domain (RBDs). When the nucleic acids or mRNA of the present invention include more than one RBD, the RBDs do not need to be from the same species or even the same structural class.

In some embodiments, at least one flanking region (e.g., the 5′-UTR and/or the 3′-UTR) may include at least one RBD. In some embodiments, the first flanking region and the second flanking region may both include at least one RBD. The RBD may be the same or each of the RBDs may have at least 60% sequence identity to the other RBD. As a non-limiting example, at least on RBD may be located before, after and/or within the 3′-UTR of the nucleic acid or mRNA of the present invention. As another non-limiting example, at least one RBD may be located before or within the first 300 nucleosides of the 3′-UTR.

In some embodiments, the nucleic acids and/or mRNA of the present invention may include at least one RBD in the first region of linked nucleosides. The RBD may be located before, after or within a coding region (e.g., the ORF).

In yet another embodiment, the first region of linked nucleosides and/or at least one flanking region may include at least on RBD. As a non-limiting example, the first region of linked nucleosides may include a RBD related to splicing factors and at least one flanking region may include a RBD for stability and/or translation factors.

In some embodiments, the nucleic acids and/or mRNA of the present invention may include at least one RBD located in a coding and/or non-coding region of the nucleic acids and/or mRNA.

In some embodiments, at least one RBD may be incorporated into at least one flanking region to increase the stability of the nucleic acid and/or mRNA of the present invention.

In some embodiments, a microRNA sequence in a RNA binding protein motif may be used to decrease the accessibility of the site of translation initiation such as, but not limited to a start codon. The nucleic acids or mRNA of the present invention may include a microRNA sequence, instead of the LNA or EJC sequence described by Matsuda et al, near the site of translation initiation in order to decrease the accessibility to the site of translation initiation. The site of translation initiation may be prior to, after or within the microRNA sequence. As a non-limiting example, the site of translation initiation may be located within a microRNA sequence such as a seed sequence or binding site. As another non-limiting example, the site of translation initiation may be located within a miR-122 sequence such as the seed sequence or the mir-122 binding site.

In some embodiments, an antisense locked nucleic acid (LNA) oligonucleotides and exon-junction complexes (EJCs) may be used in the RNA binding protein motif. The LNA and EJCs may be used around a start codon (−4 to +37 where the A of the AUG codons is +1) in order to decrease the accessibility to the first start codon (AUG).

### Codon Optimization

The polynucleotides of the invention, their regions or parts or subregions may be codon optimized. Codon optimization methods are known in the art and may be useful in efforts to achieve one or more of several goals. These goals include to match codon frequencies in target and host organisms to ensure proper folding, bias GC content to increase mRNA stability or reduce secondary structures, minimize tandem repeat codons or base runs that may impair gene construction or expression, customize transcriptional and translational control regions, insert or remove protein trafficking sequences, remove/add post translation modification sites in encoded protein (e.g., glycosylation sites), add, remove or shuffle protein domains, insert or delete restriction sites, modify ribosome binding sites and mRNA degradation sites, to adjust translational rates to allow the various domains of the protein to fold properly, or to reduce or eliminate problem secondary structures within the polynucleotide. Codon optimization tools, algorithms and services are known in the art, non-limiting examples include services from GeneArt (Life Technologies), DNA2.0 (Menlo Park Calif.) and/or proprietary methods. In some embodiments, the ORF sequence is optimized using optimization algorithms. Codon options for each amino acid are given in Table 3.

TABLE 3
Codon Options.

| | Single Letter | |
|---|---|---|
| Amino Acid | Code | Codon Options |
| Isoleucine | I | ATT, ATC, ATA |
| Leucine | L | CTT, CTC, CTA, CTG, TTA, TTG |
| Valine | V | GTT, GTC, GTA, GTG |
| Phenylalanine | F | TTT, TTC |
| Methionine | M | ATG |
| Cysteine | C | TGT, TGC |
| Alanine | A | GCT, GCC, GCA, GCG |
| Glycine | G | GGT, GGC, GGA, GGG |
| Proline | P | CCT, CCC, CCA, CCG |
| Threonine | T | ACT, ACC, ACA, ACG |
| Serine | S | TCT, TCC, TCA, TCG, AGT, AGC |
| Tyrosine | Y | TAT, TAC |
| Tryptophan | W | TGG |
| Glutamine | Q | CAA, CAG |
| Asparagine | N | AAT, AAC |
| Histidine | H | CAT, CAC |
| Glutamic acid | E | GAA, GAG |
| Aspartic acid | D | GAT, GAC |
| Lysine | K | AAA, AAG |
| Arginine | R | CGT, CGC, CGA, CGG, AGA, AGG |
| Selenocysteine | Sec | UGA in mRNA in presence of Selenocystein |
| | | insertion element (SECIS) |
| Stop codons | Stop | TAA, TAG, TGA |

“Codon optimized” refers to the modification of a starting nucleotide sequence by replacing at least one codon of the starting nucleotide sequence with a codon that is more frequently used in the group of abundant polypeptides of the host organism. Table 4 contains the codon usage frequency for C humans (Codon usage database: www.kazusa.or.jp/codon/cgi-bin/showcodon.cgi?species=9606&aa=1&style=N).

Codon optimization may be used to increase the expression of polypeptides by the replacement of at least one, at least two, at least three, at least four, at least five, at least six, at least seven, at least eight, at least nine, at least ten or at least 1%, at least 2%, at least 4%, at least 6%, at least 8%, at least 10%, at least 20%, at least 40%, at least 60%, at least 80%, at least 90% or at least 95%, or all codons of the starting nucleotide sequence with more frequently or the most frequently used codons for the respective amino acid as determined for the group of abundant proteins.

In some embodiments of the invention, the nucleotide sequence of the mRNA contains for each amino acid the most frequently used codons of the abundant proteins of the respective host cell.

TABLE 4
Codon usage frequency table for humans.

| | Amino | | | Amino | | | Amino | | | Amino | |
|---|---|---|---|---|---|---|---|---|---|---|---|
| Codon | Acid | % | Codon | Acid | % | Codon | Acid | % | Codon | Acid | % |
| UUU | F | 46 | UCU | S | 19 | UAU | Y | 44 | UGU | C | 46 |
| UUC | F | 54 | UCC | S | 22 | UAC | Y | 56 | UGC | C | 54 |
| UUA | L | 8 | UCA | S | 15 | UAA | * | 30 | UGA | * | 47 |
| UUG | L | 13 | UCG | S | 5 | UAG | * | 24 | UGG | W | 100 |
| CUU | L | 13 | CCU | P | 29 | CAU | H | 42 | CGU | R | 8 |
| CUC | L | 20 | CCC | P | 32 | CAC | H | 58 | CGC | R | 18 |
| CUA | L | 7 | CCA | P | 28 | CAA | Q | 27 | CGA | R | 11 |
| CUG | L | 40 | CCG | P | 11 | CAG | Q | 73 | CGG | R | 20 |
| AUU | I | 36 | ACU | T | 25 | AAU | N | 47 | AGU | S | 15 |
| AUC | I | 47 | ACC | T | 36 | AAC | N | 53 | AGC | S | 24 |
| AUA | I | 17 | ACA | T | 28 | AAA | K | 43 | AGA | R | 21 |
| AUG | M | 100 | ACG | T | 11 | AAG | K | 57 | AGG | R | 21 |
| GUU | V | 18 | GCU | A | 27 | GAU | D | 46 | GGU | G | 16 |
| GUC | V | 24 | GCC | A | 40 | GAC | D | 54 | GGC | G | 34 |
| GUA | V | 12 | GCA | A | 23 | GAA | E | 42 | GGA | G | 25 |
| GUG | V | 46 | GCG | A | 11 | GAG | E | 58 | GGG | G | 25 |

In some embodiments, after a nucleotide sequence has been codon optimized it may be further evaluated for regions containing restriction sites. At least one nucleotide within the restriction site regions may be replaced with another nucleotide in order to remove the restriction site from the sequence but the replacement of nucleotides does alter the amino acid sequence which is encoded by the codon optimized nucleotide sequence.

Features, which may be considered beneficial in some embodiments of the present invention, may be encoded by regions of the polynucleotide and such regions may be upstream (5′) or downstream (3′) to a region which encodes a polypeptide. These regions may be incorporated into the polynucleotide before and/or after codon optimization of the protein encoding region or open reading frame (ORF). It is not required that a polynucleotide contain both a 5′ and 3′ flanking region. Examples of such features include, but are not limited to, untranslated regions (UTRs), Kozak sequences, an oligo(dT) sequence, and detectable tags and may include multiple cloning sites which may have XbaI recognition.

In some embodiments, a 5′-UTR and/or a 3′-UTR region may be provided as flanking regions. Multiple 5′- or 3′-UTRs may be included in the flanking regions and may be the same or of different sequences. Any portion of the flanking regions, including none, may be codon optimized and any may independently contain one or more different structural or chemical alterations, before and/or after codon optimization.

After optimization (if desired), the polynucleotides components are reconstituted and transformed into a vector such as, but not limited to, plasmids, viruses, cosmids, and artificial chromosomes. For example, the optimized polynucleotide may be reconstituted and transformed into chemically competent E. coli, yeast, neurospora, maize, drosophila, etc. where high copy plasmid-like or chromosome structures occur by methods described herein.

### Uses of Compositions

Therapeutic Agents

The compositions described herein can be used as therapeutic agents. For example, a composition described herein can be administered to a subject (e.g., an animal or a human subject), wherein the mRNA of the composition is translated in vivo to produce a therapeutic peptide in the subject. Accordingly, provided herein are compositions, including pharmaceutical compositions, methods, kits, and reagents for treatment or prevention of disease or conditions in humans and other mammals. The active therapeutic agents of the present disclosure include any one of the compositions described herein, cells containing or cells contacts with any one of the composition described herein, polypeptides translated from any one of the compositions described herein, tissues containing cells containing any one of the compositions described herein, or organs containing tissues containing cells containing any one of the compositions described herein.

Provided are methods of inducing translation of a synthetic or recombinant polynucleotide to produce a polypeptide in a cell population using the compositions described herein. Such translation can be in vivo, ex vivo, in culture, or in vitro. The cell population is contacted with an effective amount of a composition described herein. The population is contacted under conditions such that the nucleic acid is localized into one or more cells of the cell population and the recombinant polypeptide is translated in the cell from the nucleic acid.

An effective amount of the composition is provided based, at least in part, on the target tissue, target cell type, means of administration, physical characteristics of the nucleic acid (e.g., size, and extent of alternative nucleosides), and other determinants. In general, an effective amount of the composition provides efficient protein production in the cell, preferably more efficient than a composition containing a corresponding unaltered nucleic acid. Increased efficiency may be demonstrated by increased cell transfection (i.e., the percentage of cells transfected with the nucleic acid), increased protein translation from the nucleic acid, decreased nucleic acid degradation (as demonstrated, e.g., by increased duration of protein translation from a modified nucleic acid), or reduced innate immune response of the host cell or improve therapeutic utility.

Aspects of the present disclosure are directed to methods of inducing in vivo translation of a recombinant polypeptide in a mammalian subject (e.g., a human subject) in need thereof. The composition is provided in an amount and under other conditions such that the mRNA is localized into a cell or cells of the subject and the recombinant polypeptide is translated in the cell from the mRNA. The cell in which the mRNA is localized, or the tissue in which the cell is present, may be targeted with one or more than one rounds of administration.

Other aspects of the present disclosure relate to transplantation of cells containing a composition of the invention to a mammalian subject. Administration of cells to mammalian subjects is known to those of ordinary skill in the art, such as local implantation (e.g., topical or subcutaneous administration), organ delivery or systemic injection (e.g., intravenous injection or inhalation), as is the formulation of cells in pharmaceutically acceptable carrier. Pharmaceutical compositions containing composition of the invention are formulated for administration intramuscularly, transarterially, intraperitoneally, intravenously, intranasally, subcutaneously, endoscopically, transdermally, or intrathecally. In some embodiments, the composition is formulated for extended release.

The subject to whom the therapeutic agent is administered suffers from or is at risk of developing a disease, disorder, or deleterious condition. Provided are methods of identifying, diagnosing, and classifying subjects on these bases, which may include clinical diagnosis, biomarker levels, genome-wide association studies (GWAS), and other methods known in the art.

In certain embodiments, the administered composition directs production of one or more recombinant polypeptides that provide a functional activity which is substantially absent in the cell in which the recombinant polypeptide is translated. For example, the missing functional activity may be enzymatic, structural, or gene regulatory in nature.

In other embodiments, the administered composition directs production of one or more recombinant polypeptides that replace a polypeptide (or multiple polypeptides) that is substantially absent in the cell in which the recombinant polypeptide is translated. Such absence may be due to genetic mutation of the encoding gene or regulatory pathway thereof. In other embodiments, the administered composition directs production of one or more recombinant polypeptides to supplement the amount of polypeptide (or multiple polypeptides) that is present in the cell in which the recombinant polypeptide is translated. Alternatively, the recombinant polypeptide functions to antagonize the activity of an endogenous protein present in, on the surface of, or secreted from the cell. Usually, the activity of the endogenous protein is deleterious to the subject, for example, due to mutation of the endogenous protein resulting in altered activity or localization. Additionally, the recombinant polypeptide antagonizes, directly or indirectly, the activity of a biological moiety present in, on the surface of, or secreted from the cell. Antagonized biological moieties include lipids (e.g., cholesterol), a lipoprotein (e.g., low density lipoprotein), a nucleic acid, a carbohydrate, or a small molecule toxin.

The recombinant proteins described herein may be engineered for localization within the cell, potentially within a specific compartment such as the nucleus, or are engineered for secretion from the cell or translocation to the plasma membrane of the cell.

As described herein, a useful feature of the compositions of the present disclosure is the capacity to reduce, evade, avoid or eliminate the innate immune response of a cell to an exogenous nucleic acid. Provided are methods for performing the titration, reduction or elimination of the immune response in a cell or a population of cells. In some embodiments, the cell is contacted with a first composition that contains a first dose of a first exogenous nucleic acid including a translatable region and at least one nucleoside alteration, and the level of the innate immune response of the cell to the first exogenous nucleic acid is determined. Subsequently, the cell is contacted with a second composition, which includes a second dose of the first exogenous nucleic acid, the second dose containing a lesser amount of the first exogenous nucleic acid as compared to the first dose. Alternatively, the cell is contacted with a first dose of a second exogenous nucleic acid. The second exogenous nucleic acid may contain one or more alternative nucleosides, which may be the same or different from the first exogenous nucleic acid or, alternatively, the second exogenous nucleic acid may not contain alternative nucleosides. The steps of contacting the cell with the first composition and/or the second composition may be repeated one or more times. Additionally, efficiency of protein production (e.g., protein translation) in the cell is optionally determined, and the cell may be re-transfected with the first and/or second composition repeatedly until a target protein production efficiency is achieved.

Diseases and Conditions

Provided are methods for treating or preventing a symptom of diseases characterized by missing or aberrant protein activity, by replacing the missing protein activity or overcoming the aberrant protein activity. Because of the rapid initiation of protein production following introduction of mRNAs, as compared to viral DNA vectors, the compounds of the present disclosure are particularly advantageous in treating acute diseases such as sepsis, stroke, and myocardial infarction.

Moreover, the lack of transcriptional regulation of the mRNAs of the present disclosure is advantageous in that accurate titration of protein production is achievable. Multiple diseases are characterized by missing (or substantially diminished such that proper protein function does not occur) protein activity. Such proteins may not be present, are present in very low quantities or are essentially non-functional. The present disclosure provides a method for treating such conditions or diseases in a subject by introducing nucleic acid or cell-based therapeutics containing the compositions provided herein, wherein the compositions encode for a protein that replaces the protein activity missing from the target cells of the subject.

Diseases characterized by dysfunctional or aberrant protein activity include, but not limited to, cancer and proliferative diseases, genetic diseases (e.g., cystic fibrosis), autoimmune diseases, diabetes, neurodegenerative diseases, cardiovascular diseases, and metabolic diseases. The present disclosure provides a method for treating such conditions or diseases in a subject by introducing nucleic acid or cell-based therapeutics containing the compositions provided herein, wherein the compositions encode for a protein that antagonizes or otherwise overcomes the aberrant protein activity present in the cell of the subject.

Specific examples of a dysfunctional protein are the missense or nonsense mutation variants of the cystic fibrosis transmembrane conductance regulator (CFTR) gene, which produce a dysfunctional or nonfunctional, respectively, protein variant of CFTR protein, which causes cystic fibrosis.

Thus, provided are methods of treating cystic fibrosis in a mammalian subject by contacting a cell of the subject with a composition having a translatable region that encodes a functional CFTR polypeptide, under conditions such that an effective amount of the CTFR polypeptide is present in the cell. Preferred target cells are epithelial cells, such as the lung, and methods of administration are determined in view of the target tissue; i.e., for lung delivery, the RNA molecules are formulated for administration by inhalation. Therefore, in certain embodiments, the polypeptide of interest encoded by the mRNA of the invention is the CTFR polypeptide and the mRNA or pharmaceutical composition of the invention is for use in treating cystic fibrosis.

In some embodiments, the present disclosure provides a method for treating hyperlipidemia in a subject, by introducing into a cell population of the subject with an mRNA molecule encoding Sortilin, a protein recently characterized by genomic studies, thereby ameliorating the hyperlipidemia in a subject. The SORT1 gene encodes a trans-Golgi network (TGN) transmembrane protein called Sortilin. Genetic studies have shown that one of five individuals has a single nucleotide polymorphism, rs12740374, in the 1p13 locus of the SORT1 gene that predisposes them to having low levels of low-density lipoprotein (LDL) and very-low-density lipoprotein (VLDL). Each copy of the minor allele, present in about 30% of people, alters LDL cholesterol by 8 mg/dL, while two copies of the minor allele, present in about 5% of the population, lowers LDL cholesterol 16 mg/dL. Carriers of the minor allele have also been shown to have a 40% decreased risk of myocardial infarction. Functional in vivo studies in mice describes that overexpression of SORT1 in mouse liver tissue led to significantly lower LDL-cholesterol levels, as much as 80% lower, and that silencing SORT1 increased LDL cholesterol approximately 200% (Musunuru K et al. From noncoding variant to phenotype via SORT1 at the 1p13 cholesterol locus. Nature 2010; 466: 714-721). Therefore, in certain embodiments, the polypeptide of interest encoded by the mRNA of the invention is Sortilin and the mRNA or pharmaceutical composition of the invention is for use in treating hyperlipidemia.

In certain embodiments, the polypeptide of interest encoded by the mRNA of the invention is granulocyte colony-stimulating factor (GCSF), and the mRNA or pharmaceutical composition of the invention is for use in treating a neurological disease such as cerebral ischemia, or treating neutropenia, or for use in increasing the number of hematopoietic stem cells in the blood (e.g. before collection by leukapheresis for use in hematopoietic stem cell transplantation).

In certain embodiments, the polypeptide of interest encoded by the mRNA of the invention is erythropoietin (EPO), and the mRNA or pharmaceutical composition of the invention is for use in treating anemia, inflammatory bowel disease (such as Crohn's disease and/or ulcerative colitis) or myelodysplasia.

Targeting Moieties

In embodiments of the present disclosure, compositions are provided to express a protein-binding partner or a receptor on the surface of the cell, which functions to target the cell to a specific tissue space or to interact with a specific moiety, either in vivo or in vitro. Suitable protein-binding partners include antibodies and functional fragments thereof, scaffold proteins, or peptides. Additionally, compositions can be employed to direct the synthesis and extracellular localization of lipids, carbohydrates, or other biological moieties.

Methods of Cellular Nucleic Acid Delivery

Methods of the present disclosure enhance nucleic acid delivery into a cell population, in vivo, ex vivo, or in culture. For example, a cell culture containing a plurality of host cells (e.g., eukaryotic cells such as yeast or mammalian cells) is contacted with a composition of the invention. The composition may also contains a transfection reagent or other compound that increases the efficiency of enhanced nucleic acid uptake into the host cells.

The composition may delivered to a subject (e.g., a human subject) by methods known to those of skill in the art. In some embodiments, the composition is associated with (e.g., encapsulated by) a lipid nanoparticle (LNP). In some embodiments the LNP-associated composition is administered to a subject (e.g., a human subject having a disease or condition).

LNPs may be spherical with an average diameter between 10 and 1000 nanometers. Lipid nanoparticles possess a lipid core matrix that can solubilize lipophilic molecules. The lipid core is stabilized by surfactants (emulsifiers). The term lipid is used here in a broader sense and includes triglycerides (e.g. tristearin), diglycerides (e.g. glycerol bahenate), monoglycerides (e.g. glycerol monostearate), fatty acids (e.g. stearic acid), steroids (e.g. cholesterol), and waxes (e.g. cetyl palmitate). The core lipids can be fatty acids, acylglycerols, waxes, and mixtures of these surfactants. Biological membrane lipids such as phospholipids, sphingomyelins, bile salts (sodium taurocholate), and sterols (cholesterol) are utilized as stabilizers. Emulsifiers may be used to stabilize the lipid dispersion.

Pharmaceutical Compositions The present disclosure provides pharmaceutical composition including any one of the compositions described herein and a pharmaceutically-acceptable excipient. Pharmaceutical compositions may optionally include one or more additional therapeutically active substances. In accordance with some embodiments, a method of administering pharmaceutical compositions including a composition encoding one or more proteins to be delivered to a subject in need thereof is provided. In some embodiments, compositions are administered to humans.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the pharmaceutical compositions is contemplated include, but are not limited to, humans and/or other primates; mammals, including commercially relevant mammals such as cattle, pigs, horses, sheep, cats, dogs, mice, and/or rats; and/or birds, including commercially relevant birds such as chickens, ducks, geese, and/or turkeys.

Formulations of the pharmaceutical compositions described herein may be prepared by any method known or hereafter developed in the art of pharmacology. In general, such preparatory methods include the step of bringing the active ingredient into association with an excipient and/or one or more other accessory ingredients, and then, if necessary and/or desirable, shaping and/or packaging the product into a desired single- or multi-dose unit.

A pharmaceutical composition in accordance with the present disclosure may be prepared, packaged, and/or sold in bulk, as a single unit dose, and/or as a plurality of single unit doses. As used herein, a “unit dose” is discrete amount of the pharmaceutical composition including a predetermined amount of the active ingredient. The amount of the active ingredient is generally equal to the dosage of the active ingredient which would be administered to a subject and/or a convenient fraction of such a dosage such as, for example, one-half or one-third of such a dosage.

Relative amounts of the active ingredient, the pharmaceutically acceptable excipient, and/or any additional ingredients in a pharmaceutical composition in accordance with the present disclosure will vary, depending upon the identity, size, and/or condition of the subject treated and further depending upon the route by which the composition is to be administered. By way of example, the composition may include between 0.1% and 100% (w/w) active ingredient.

Pharmaceutical formulations may additionally include a pharmaceutically acceptable excipient, which, as used herein, includes any and all solvents, dispersion media, diluents, or other liquid vehicles, dispersion or suspension aids, surface active agents, isotonic agents, thickening or emulsifying agents, preservatives, solid binders, lubricants and the like, as suited to the particular dosage form desired. Remington's The Science and Practice of Pharmacy, 22^{nd} Edition, J. P. Remington, L. V. Allen (Pharmaceutical Press, Philadelphia, Pa., 2013; incorporated herein by reference) discloses various excipients used in formulating pharmaceutical compositions and known techniques for the preparation thereof. Except insofar as any conventional excipient medium is incompatible with a substance or its derivatives, such as by producing any undesirable biological effect or otherwise interacting in a deleterious manner with any other component(s) of the pharmaceutical composition, its use is contemplated to be within the scope of this present disclosure.

In some embodiments, a pharmaceutically acceptable excipient is at least 95%, at least 96%, at least 97%, at least 98%, at least 99%, or 100% pure. In some embodiments, an excipient is approved for use in humans and for veterinary use. In some embodiments, an excipient is approved by United States Food and Drug Administration. In some embodiments, an excipient is pharmaceutical grade. In some embodiments, an excipient meets the standards of the United States Pharmacopoeia (USP), the European Pharmacopoeia (EP), the British Pharmacopoeia, and/or the International Pharmacopoeia.

Pharmaceutically acceptable excipients used in the manufacture of pharmaceutical compositions include, but are not limited to, inert diluents, dispersing and/or granulating agents, surface active agents and/or emulsifiers, disintegrating agents, binding agents, preservatives, buffering agents, lubricating agents, and/or oils. Such excipients may optionally be included in pharmaceutical formulations. Excipients such as cocoa butter and suppository waxes, coloring agents, coating agents, sweetening, flavoring, and/or perfuming agents can be present in the composition, according to the judgment of the formulator.

Diluents include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and/or combinations thereof.

Granulating and/or dispersing agents include, but are not limited to, potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, etc., and/or combinations thereof.

Surface active agents and/or emulsifiers include, but are not limited to, natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite (aluminum silicate) and Veegum® (magnesium aluminum silicate)), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate (Tween®20), polyoxyethylene sorbitan (Tween®60), polyoxyethylene sorbitan monooleate (Tween®80), sorbitan monopalmitate (Span®40), sorbitan monostearate (Span®60), sorbitan tristearate (Span®65), glyceryl monooleate, sorbitan monooleate (Span®80)), polyoxyethylene esters (e.g., polyoxyethylene monostearate (Myrj®45), polyoxyethylene hydrogenated castor oil, polyethoxylated castor oil, polyoxymethylene stearate, and Solutol®), sucrose fatty acid esters, polyethylene glycol fatty acid esters (e.g., Cremophor®), polyoxyethylene ethers, (e.g., polyoxyethylene lauryl ether (Brij®30)), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic®F 68, Poloxamer®188, cetrimonium bromide, cetylpyridinium chloride, benzalkonium chloride, docusate sodium, etc. and/or combinations thereof.

Binding agents include, but are not limited to, starch (e.g., cornstarch and starch paste); gelatin; sugars (e.g., sucrose, glucose, dextrose, dextrin, molasses, lactose, lactitol, mannitol,); natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum®), and larch arabogalactan); alginates; polyethylene oxide; polyethylene glycol; inorganic calcium salts; silicic acid; polymethacrylates; waxes; water; alcohol; etc.; and combinations thereof.

Preservatives include, but are not limited to, antioxidants, chelating agents, antimicrobial preservatives, antifungal preservatives, alcohol preservatives, acidic preservatives, and/or other preservatives. Antioxidants include, but are not limited to, alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and/or sodium sulfite. Chelating agents include ethylenediaminetetraacetic acid (EDTA), citric acid monohydrate, disodium edetate, dipotassium edetate, edetic acid, fumaric acid, malic acid, phosphoric acid, sodium edetate, tartaric acid, and/or trisodium edetate. Antimicrobial preservatives include, but are not limited to, benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and/or thimerosal. Antifungal preservatives include, but are not limited to, butyl paraben, methyl paraben, ethyl paraben, propyl paraben, benzoic acid, hydroxybenzoic acid, potassium benzoate, potassium sorbate, sodium benzoate, sodium propionate, and/or sorbic acid. Alcohol preservatives include, but are not limited to, ethanol, polyethylene glycol, phenol, phenolic compounds, bisphenol, chlorobutanol, hydroxybenzoate, and/or phenylethyl alcohol. Acidic preservatives include, but are not limited to, vitamin A, vitamin C, vitamin E, beta-carotene, citric acid, acetic acid, dehydroacetic acid, ascorbic acid, sorbic acid, and/or phytic acid. Other preservatives include, but are not limited to, tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant Plus®, Phenonip®, methylparaben, Germall®115, Germaben®II, Neolone™, Kathon™, and/or Euxyl®.

Buffering agents include, but are not limited to, citrate buffer solutions, acetate buffer solutions, phosphate buffer solutions, ammonium chloride, calcium carbonate, calcium chloride, calcium citrate, calcium glubionate, calcium gluceptate, calcium gluconate, d-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, etc., and/or combinations thereof.

Lubricating agents include, but are not limited to, magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, etc., and combinations thereof.

Oils include, but are not limited to, almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and/or combinations thereof.

Liquid dosage forms for oral and parenteral administration include, but are not limited to, pharmaceutically acceptable emulsions, microemulsions, solutions, suspensions, syrups, and/or elixirs. In addition to active ingredients, liquid dosage forms may include inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, oral compositions can include adjuvants such as wetting agents, emulsifying and suspending agents, sweetening, flavoring, and/or perfuming agents. In certain embodiments for parenteral administration, compositions are mixed with solubilizing agents such as Cremophor®, alcohols, oils, modified oils, glycols, polysorbates, cyclodextrins, polymers, and/or combinations thereof.

Injectable preparations, for example, sterile injectable aqueous or oleaginous suspensions may be formulated according to the known art using suitable dispersing agents, wetting agents, and/or suspending agents. Sterile injectable preparations may be sterile injectable solutions, suspensions, and/or emulsions in nontoxic parenterally acceptable diluents and/or solvents, for example, as a solution in 1,3-butanediol. Among the acceptable vehicles and solvents that may be employed are water, Ringer's solution, U.S.P., and isotonic sodium chloride solution. Sterile, fixed oils are conventionally employed as a solvent or suspending medium. For this purpose any bland fixed oil can be employed including synthetic mono- or diglycerides. Fatty acids such as oleic acid can be used in the preparation of injectables.

Injectable formulations can be sterilized, for example, by filtration through a bacterial-retaining filter, and/or by incorporating sterilizing agents in the form of sterile solid compositions which can be dissolved or dispersed in sterile water or other sterile injectable medium prior to use.

In order to prolong the effect of an active ingredient, it is often desirable to slow the absorption of the active ingredient from subcutaneous or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle. Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are prepared by entrapping the drug in liposomes or microemulsions which are compatible with body tissues.

Compositions for rectal or vaginal administration are typically suppositories which can be prepared by mixing compositions with suitable non-irritating excipients such as cocoa butter, polyethylene glycol or a suppository wax which are solid at ambient temperature but liquid at body temperature and therefore melt in the rectum or vaginal cavity and release the active ingredient.

Solid dosage forms for oral administration include capsules, tablets, pills, powders, and granules. In such solid dosage forms, an active ingredient is mixed with at least one inert, pharmaceutically acceptable excipient such as sodium citrate or dicalcium phosphate and/or fillers or extenders (e.g., starches, lactose, sucrose, glucose, mannitol, and silicic acid), binders (e.g., carboxymethylcellulose, alginates, gelatin, polyvinylpyrrolidinone, sucrose, and acacia), humectants (e.g., glycerol), disintegrating agents (e.g., agar, calcium carbonate, potato or tapioca starch, alginic acid, certain silicates, and sodium carbonate), solution retarding agents (e.g., paraffin), absorption accelerators (e.g., quaternary ammonium compounds), wetting agents (e.g. cetyl alcohol and glycerol monostearate), absorbents (e.g. kaolin and bentonite clay), and lubricants (e.g., talc, calcium stearate, magnesium stearate, solid polyethylene glycols, sodium lauryl sulfate), and mixtures thereof. In the case of capsules, tablets and pills, the dosage form may include buffering agents.

Solid compositions of a similar type may be employed as fillers in soft and hard-filled gelatin capsules using such excipients as lactose or milk sugar as well as high molecular weight polyethylene glycols and the like. Solid dosage forms of tablets, dragees, capsules, pills, and granules can be prepared with coatings and shells such as enteric coatings and other coatings well known in the pharmaceutical formulating art. They may optionally include opacifying agents and can be of a composition that they release the active ingredient(s) only, or preferentially, in a certain part of the intestinal tract, optionally, in a delayed manner. Examples of embedding compositions which can be used include polymeric substances and waxes. Solid compositions of a similar type may be employed as fillers in soft and hard-filled gelatin capsules using such excipients as lactose or milk sugar as well as high molecular weight polyethylene glycols and the like.

Dosage forms for topical and/or transdermal administration of a composition may include ointments, pastes, creams, lotions, gels, powders, solutions, sprays, inhalants and/or patches. Generally, an active ingredient is admixed under sterile conditions with a pharmaceutically acceptable excipient and/or any needed preservatives and/or buffers as may be required. Additionally, the present disclosure contemplates the use of transdermal patches, which often have the added advantage of providing controlled delivery of a compound to the body. Such dosage forms may be prepared, for example, by dissolving and/or dispensing the compound in the proper medium. Alternatively or additionally, rate may be controlled by either providing a rate controlling membrane and/or by dispersing the compound in a polymer matrix and/or gel.

Suitable devices for use in delivering intradermal pharmaceutical compositions described herein include short needle devices such as those described in U.S. Pat. Nos. 4,886,499; 5,190,521; 5,328,483; 5,527,288; 4,270,537; 5,015,235; 5,141,496; and 5,417,662. Intradermal compositions may be administered by devices which limit the effective penetration length of a needle into the skin, such as those described in International Patent Publication No. WO199934850 and functional equivalents thereof. Jet injection devices which deliver liquid compositions to the dermis via a liquid jet injector and/or via a needle which pierces the stratum corneum and produces a jet which reaches the dermis are suitable. Jet injection devices are described, for example, in U.S. Pat. Nos. 5,480,381; 5,599,302; 5,334,144; 5,993,412; 5,649,912; 5,569,189; 5,704,911; 5,383,851; 5,893,397; 5,466,220; 5,339,163; 5,312,335; 5,503,627; 5,064,413; 5,520,639; 4,596,556; 4,790,824; 4,941,880; 4,940,460; and International Patent Publication Nos. WO1997/37705 and WO1997/13537. Ballistic powder/particle delivery devices which use compressed gas to accelerate vaccine in powder form through the outer layers of the skin to the dermis are suitable. Alternatively or additionally, conventional syringes may be used in the classical mantoux method of intradermal administration.

Formulations suitable for topical administration include, but are not limited to, liquid and/or semi liquid preparations such as liniments, lotions, oil in water and/or water in oil emulsions such as creams, ointments and/or pastes, and/or solutions and/or suspensions. Topically-administrable formulations may, for example, include from about 1% to about 10% (w/w) active ingredient, although the concentration of active ingredient may be as high as the solubility limit of the active ingredient in the solvent. Formulations for topical administration may further include one or more of the additional ingredients described herein.

A pharmaceutical composition may be prepared, packaged, and/or sold in a formulation suitable for pulmonary administration via the buccal cavity. Such a formulation may include dry particles which include the active ingredient and which have a diameter in the range from about 0.5 nm to about 7 nm or from about 1 nm to about 6 nm. Such compositions are conveniently in the form of dry powders for administration using a device including a dry powder reservoir to which a stream of propellant may be directed to disperse the powder and/or using a self-propelling solvent/powder dispensing container such as a device including the active ingredient dissolved and/or suspended in a low-boiling propellant in a sealed container. Such powders include particles wherein at least 98% of the particles by weight have a diameter greater than 0.5 nm and at least 95% of the particles by number have a diameter less than 7 nm. Alternatively, at least 95% of the particles by weight have a diameter greater than 1 nm and at least 90% of the particles by number have a diameter less than 6 nm. Dry powder compositions may include a solid fine powder diluent such as sugar and are conveniently provided in a unit dose form.

Low boiling propellants generally include liquid propellants having a boiling point of below 65° F. at atmospheric pressure. Generally the propellant may constitute 50% to 99.9% (w/w) of the composition, and active ingredient may constitute 0.1% to 20% (w/w) of the composition. A propellant may further include additional ingredients such as a liquid non-ionic and/or solid anionic surfactant and/or a solid diluent (which may have a particle size of the same order as particles including the active ingredient).

Pharmaceutical compositions formulated for pulmonary delivery may provide an active ingredient in the form of droplets of a solution and/or suspension. Such formulations may be prepared, packaged, and/or sold as aqueous and/or dilute alcoholic solutions and/or suspensions, optionally sterile, including active ingredient, and may conveniently be administered using any nebulization and/or atomization device. Such formulations may further include one or more additional ingredients including, but not limited to, a flavoring agent such as saccharin sodium, a volatile oil, a buffering agent, a surface active agent, and/or a preservative such as methylhydroxybenzoate. Droplets provided by this route of administration may have an average diameter in the range from about 0.1 nm to about 200 nm.

Formulations described herein as being useful for pulmonary delivery are useful for intranasal delivery of a pharmaceutical composition. Another formulation suitable for intranasal administration is a coarse powder including the active ingredient and having an average particle from about 0.2 μm to 500 μm. Such a formulation is administered in the manner in which snuff is taken, i.e. by rapid inhalation through the nasal passage from a container of the powder held close to the nose.

Formulations suitable for nasal administration may, for example, include from about as little as 0.1% (w/w) and as much as 100% (w/w) of active ingredient, and may include one or more of the additional ingredients described herein. A pharmaceutical composition may be prepared, packaged, and/or sold in a formulation suitable for buccal administration. Such formulations may, for example, be in the form of tablets and/or lozenges made using conventional methods, and may, for example, 0.1% to 20% (w/w) active ingredient, the balance including an orally dissolvable and/or degradable composition and, optionally, one or more of the additional ingredients described herein. Alternately, formulations suitable for buccal administration may include a powder and/or an aerosolized and/or atomized solution and/or suspension including active ingredient. Such powdered, aerosolized, and/or aerosolized formulations, when dispersed, may have an average particle and/or droplet size in the range from about 0.1 nm to about 200 nm, and may further include one or more of any additional ingredients described herein.

A pharmaceutical composition may be prepared, packaged, and/or sold in a formulation suitable for ophthalmic administration. Such formulations may, for example, be in the form of eye drops including, for example, a 0.1/1.0% (w/w) solution and/or suspension of the active ingredient in an aqueous or oily liquid excipient. Such drops may further include buffering agents, salts, and/or one or more other of any additional ingredients described herein. Other ophthalmically-administrable formulations which are useful include those which include the active ingredient in microcrystalline form and/or in a liposomal preparation. Ear drops and/or eye drops are contemplated as being within the scope of this present disclosure.

General considerations in the formulation and/or manufacture of pharmaceutical agents may be found, for example, in Remington's The Science and Practice of Pharmacy, 22^{nd} Edition, J. P. Remington, L. V. Allen, Pharmaceutical Press, Philadelphia, Pa., 2013 (incorporated herein by reference).

### Administration

The present disclosure provides methods including administering a composition described herein to a subject in need thereof. A composition described herein may be administered to a subject using any amount and any route of administration effective for preventing, treating, diagnosing, or imaging a disease, disorder, and/or condition. The exact amount required will vary from subject to subject, depending on the species, age, and general condition of the subject, the severity of the disease, the particular composition, its mode of administration, its mode of activity, and the like. Compositions in accordance with the present disclosure are typically formulated in dosage unit form for ease of administration and uniformity of dosage. It will be understood, however, that the total daily usage of the compositions of the present disclosure will be decided by the attending physician within the scope of sound medical judgment. The specific therapeutically effective, prophylactically effective, or appropriate imaging dose level for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

Compositions described herein may be administered to animals, such as mammals (e.g., humans, domesticated animals, cats, dogs, mice, rats, etc.). In some embodiments, pharmaceutical, prophylactic, diagnostic, or imaging compositions thereof are administered to humans.

Compositions described herein may be administered by any route. In some embodiments, proteins and/or pharmaceutical, prophylactic, diagnostic, or imaging compositions thereof, are administered by one or more of a variety of routes, including oral, intravenous, intramuscular, intra-arterial, intramedullary, intrathecal, subcutaneous, intraventricular, transdermal, interdermal, rectal, intravaginal, intraperitoneal, topical (e.g., by powders, ointments, creams, gels, lotions, and/or drops), mucosal, nasal, buccal, enteral, vitreal, intratumoral, sublingual; by intratracheal instillation, bronchial instillation, and/or inhalation; as an oral spray, nasal spray, and/or aerosol, and/or through a portal vein catheter. In some embodiments the composition is administered by systemic intravenous injection. In specific embodiments the composition is administered intravenously and/or orally.

In certain embodiments, compositions in accordance with the present disclosure may be administered at dosage levels sufficient to deliver from about 0.0001 mg/kg to about 100 mg/kg, from about 0.01 mg/kg to about 50 mg/kg, from about 0.1 mg/kg to about 40 mg/kg, from about 0.5 mg/kg to about 30 mg/kg, from about 0.01 mg/kg to about 10 mg/kg, from about 0.1 mg/kg to about 10 mg/kg, or from about 1 mg/kg to about 25 mg/kg, of subject body weight per day, one or more times a day, to obtain the desired therapeutic, diagnostic, prophylactic, or imaging effect. The desired dosage may be delivered three times a day, two times a day, once a day, every other day, every third day, every week, every two weeks, every three weeks, or every four weeks. In certain embodiments, the desired dosage may be delivered using multiple administrations (e.g., two, three, four, five, six, seven, eight, nine, ten, eleven, twelve, thirteen, fourteen, or more administrations).

Compositions described herein may be used in combination with one or more other therapeutic, prophylactic, diagnostic, or imaging agents. By “in combination with,” it is not intended to imply that the agents must be administered at the same time and/or formulated for delivery together, although these methods of delivery are within the scope of the present disclosure. Compositions can be administered concurrently with, prior to, or subsequent to, one or more other desired therapeutics or medical procedures. In general, each agent will be administered at a dose and/or on a time schedule determined for that agent. In some embodiments, the present disclosure encompasses the delivery of pharmaceutical, prophylactic, diagnostic, or imaging compositions in combination with agents that improve their bioavailability, reduce and/or modify their metabolism, inhibit their excretion, and/or modify their distribution within the body.

It will further be appreciated that therapeutically, prophylactically, diagnostically, or imaging active agents utilized in combination may be administered together in a single composition or administered separately in different compositions. In general, it is expected that agents utilized in combination with be utilized at levels that do not exceed the levels at which they are utilized individually. In some embodiments, the levels utilized in combination will be lower than those utilized individually.

The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, a composition useful for treating cancer in accordance with the present disclosure may be administered concurrently with a chemotherapeutic agent), or they may achieve different effects (e.g., control of any adverse effects).

### Definitions

To facilitate the understanding of this invention, a number of terms are defined below and throughout the disclosure. Unless otherwise defined, all technical and scientific terms used herein have the same meaning as commonly understood by one of ordinary skill in the art to which this invention belongs. The terminology herein is used to describe specific embodiments of the invention, but their usage does not limit the invention, except as outlined in the claims.

At various places in the present specification, substituents of compounds of the present disclosure are disclosed in groups or in ranges. It is specifically intended that the present disclosure include each and every individual subcombination of the members of such groups and ranges. For example, the term “C₁₋₆ alkyl” is specifically intended to individually disclose methyl, ethyl, C₃ alkyl, C₄ alkyl, C₅ alkyl, and C₆ alkyl.

Terms such as “a”, “an,” and “the” are not intended to refer to only a singular entity, but include the general class of which a specific example may be used for illustration.

As used herein, the term “about” refers to a value that is within 10% above or below the value being described.

The term “nucleic acid” or “polynucleotide” includes any compound and/or substance that includes a chain of two or more linked nucleosides. Exemplary nucleic acids for use in accordance with the present disclosure include, but are not limited to, one or more of DNA, RNA including messenger mRNA (mRNA), hybrids thereof, RNAi-inducing agents, RNAi agents, siRNAs, shRNAs, miRNAs, antisense RNAs, ribozymes, catalytic DNA, RNAs that induce triple helix formation, aptamers, vectors, etc., described in detail herein. An oligonucleotide is a polynucleotide including 4 or more linked nucleosides. Nucleosides may include alternative nucleobases, sugar modifications, or internucleoside linkages as described herein.

The term “polypeptide” as used herein refers to a string of at least two amino acids attached to one another by a peptide bond. In some embodiments, a polypeptide may include at least 3-5 amino acids, each of which is attached to others by way of at least one peptide bond. Those of ordinary skill in the art will appreciate that polypeptides can include one or more “non-natural” amino acids or other entities that nonetheless are capable of integrating into a polypeptide chain. In some embodiments, a polypeptide may be glycosylated, e.g., a polypeptide may contain one or more covalently linked sugar moieties. In some embodiments, a single “polypeptide” (e.g., an antibody polypeptide) may comprise two or more individual polypeptide chains, which may in some cases be linked to one another, for example by one or more disulfide bonds or other means. Polypeptides of the invention include proteins, such as proteins associated with a disease or condition.

The term “innate immune response” includes a cellular response to exogenous single stranded nucleic acids, generally of viral or bacterial origin, which involves the induction of cytokine expression and release, particularly the interferons, and cell death. Protein synthesis is also reduced during the innate immune response. An innate immune response can be measured by expression or activity level of Type 1 interferons or the expression of interferon-regulated genes such as the toll-like receptors (e.g., TLR7 and TLR8). Reduction or lack of induction of innate immune response can also be measured by decreased cell death.

The term “translational enhancer element” or “translation enhancer element” (herein collectively referred to as “TEE”) refers to sequences that increase the amount of polypeptide or protein produced from an mRNA.

As used herein, the term “microRNA site” refers to a microRNA target site or a microRNA recognition site, or any nucleotide sequence to which a microRNA binds or associates. It should be understood that “binding” may follow traditional Watson-Crick hybridization rules or may reflect any stable association of the microRNA with the target sequence at or adjacent to the microRNA site.

As used herein, the terms “associated with,” “conjugated,” “linked,” “attached,” and “tethered,” when used with respect to two or more moieties, means that the moieties are physically associated or connected with one another, either directly or via one or more additional moieties that serves as a linking agent, to form a structure that is sufficiently stable so that the moieties remain physically associated under the conditions in which the structure is used, e.g., physiological conditions. An “association” need not be strictly through direct covalent chemical bonding. It may also suggest ionic or hydrogen bonding or a hybridization based connectivity sufficiently stable such that the “associated” entities remain physically associated.

The term “subject,” as used herein, can be a human, non-human primate, or other mammal, such as but not limited to dog, cat, horse, cow, pig, turkey, goat, fish, monkey, chicken, rat, mouse, and sheep.

The term “treating” or “to treat,” as used herein, refers to a therapeutic treatment of a disease or condition in a subject. In some embodiments, a therapeutic treatment may slow the progression of the disease or condition, decrease the severity of the symptoms associated with the disease or condition, improve the subject's outcome, and/or cure the disease or condition. In some embodiments, a therapeutic treatment in a subject may alleviate or ameliorate of one or more symptoms or conditions associated with the disease or condition, stabilize (i.e., not worsening) the state of the disease or condition, prevent the spread of the disease or condition, and/or delay or slow the progress of the disease or condition, as compare the state and/or the state of the disease or condition in the absence of the therapeutic treatment.

The term “therapeutically effective amount,” as used herein, refers to an amount, e.g., pharmaceutical dose, effective in inducing a desired effect in a subject or in treating a subject having a condition or disorder described herein. It is also to be understood herein that a “therapeutically effective amount” may be interpreted as an amount giving a desired therapeutic and/or preventative effect, taken in one or more doses or in any dosage or route, and/or taken alone or in combination with other therapeutic agents. For example, in the context of administering a composition described herein that is used for the treatment of a disorder or condition, an effective amount of a compound is, for example, an amount sufficient to prevent, slow down, or reverse the progression of the disorder or condition as compared to the response obtained without administration of the compound.

As used herein, the term “pharmaceutically acceptable carrier” refers to an excipient or diluent in a pharmaceutical composition. For example, a pharmaceutically acceptable carrier may be a vehicle capable of suspending or dissolving the active compound (e.g., a composition described herein). The pharmaceutically acceptable carrier must be compatible with the other ingredients of the formulation and not deleterious to the recipient. In the present disclosure, the pharmaceutically acceptable carrier must provide adequate pharmaceutical stability to a compound described herein. The nature of the carrier differs with the mode of administration. For example, for oral administration, a solid carrier is preferred; for intravenous administration, an aqueous solution carrier (e.g., WFI, and/or a buffered solution) is generally used.

As used herein, the term “conjugate” refers to a compound formed by the joining (e.g., via a covalent bond forming reaction) of two or more chemical compounds (e.g., one or more oligonucleotides and a linker, and/or one or more oligonucleotides, a linker, and a moiety).

As used herein, the term “stem-loop” refers to a base pairing pattern that can occur in single-stranded nucleic acids. The structure is also known as a hairpin or hairpin loop. It occurs when two regions of the same strand, usually complementary in nucleotide sequence when read in opposite directions, base-pair to form a double helix that ends in an unpaired loop. The resulting structure is a key building block of many RNA secondary structures. In some embodiments, each strand of the stem includes 3 to 100 nucleotides (e.g., 3-5, 5-10, 10-20, 20-30, 30-40, 40-50, or 50-100 nucleotides). In some embodiments, the unpaired loop includes 3 to 100 nucleotides (e.g., 3-5, 5-10, 10-20, 20-30, 30-40, 40-50, or 50-100 nucleotides).

As used herein, the term “triple helix” refers to an oligonucleotide structure, wherein three oligonucleotide strands wind around each other to form a set of three congruent geometrical helices with the same axis, differing by a translation along the axis (e.g., a helix having three strands). In some embodiments, each strand of the helix includes 3 to 200 nucleotides (e.g., 3-5, 5-10, 10-20, 20-30, 30-40, 40-50, 50-100, 100-150, or 150-200 nucleotides).

As used herein, the term “compaction” (e.g., as it relates to a nucleic acid, such as an mRNA) refers to a decrease in the size, volume, or length of a nucleic acid. mRNA compaction can be determined by standard techniques known to those of skill in the art. For example, mRNA compaction can be determined by maximum ladder distance (MLD). MLD is the longest chain of edges that can be drawn within a diagram depicting the predicted most energetically stable secondary structure of a nucleic acid. MLD can be determined according to methods known to those of skill in the art, for example, as described in Borodavka et al. Sizes of long RNA molecules are determined by the branching patterns of their secondary structures. Biophysical Journal 111(10):2077-2085, 2016, which is hereby incorporated by reference in its entirety.

As used herein, any values provided in a range of values include both the upper and lower bounds, and any values contained within the upper and lower bounds.

### EXAMPLES

The following examples are put forth so as to provide those of ordinary skill in the art with a description of how the compositions and methods described herein may be used, made, and evaluated, and are intended to be purely exemplary of the invention and are not intended to limit the scope of what the inventors regard as their invention.

### Example 1: PCR for cDNA Production

PCR procedures for the preparation of cDNA are performed using 2×KAPA HIFI™ HotStart ReadyMix by Kapa Biosystems (Woburn, Mass.). This system includes 2×KAPA ReadyMix12.5 μl; Forward Primer (10 uM) 0.75 μl; Reverse Primer (10 uM) 0.75 μl; Template cDNA 100 ng; and dH₂0 diluted to 25.0 μl. The reaction conditions are at 95° C. for 5 min. and 25 cycles of 98° C. for 20 sec, then 58° C. for 15 sec, then 72° C. for 45 sec, then 72° C. for 5 min. then 4° C. to termination.

The reverse primer of the instant invention incorporates a poly-T₁₂₀ (SEQ ID NO: 1) for a poly-A₁₂₀ (SEQ ID NO: 2) in the mRNA. Other reverse primers with longer or shorter poly-T tracts can be used to adjust the length of the poly-A tail in the mRNA.

The reaction is cleaned up using Invitrogen's PURELINK™ PCR Micro Kit (Carlsbad, Calif.) per manufacturer's instructions (up to 5 μg). Larger reactions will require a cleanup using a product with a larger capacity. Following the cleanup, the cDNA is quantified using the NanoDrop and analyzed by agarose gel electrophoresis to confirm the cDNA is the expected size. The cDNA is then submitted for sequencing analysis before proceeding to the in vitro transcription reaction.

### Example 2. In Vitro Transcription (IVT)

A. Materials and Methods

mRNAs according to the invention are made using standard laboratory methods and materials for in vitro transcription with the exception that the nucleotide mix contains alternative nucleotides. The open reading frame (ORF) of the gene of interest may be flanked by a 5′ untranslated region (UTR) containing a strong Kozak translational initiation signal and an alpha-globin 3′ UTR terminating with an oligo(dT) sequence for templated addition of a polyA tail for mRNAs not incorporating adenosine analogs. Adenosine-containing mRNAs are synthesized without an oligo (dT) sequence to allow for post-transcription poly (A) polymerase poly-(A) tailing.

The ORF may also include various upstream or downstream additions (such as, but not limited to, β-globin, tags, etc.) may be ordered from an optimization service such as, but limited to, DNA2.0 (Menlo Park, Calif.) and may contain multiple cloning sites which may have XbaI recognition. Upon receipt of the construct, it may be reconstituted and transformed into chemically competent E. coli.

For the present invention, NEB DH5-alpha Competent E. coli may be used. Transformations are performed according to NEB instructions using 100 ng of plasmid. The protocol is as follows:

Thaw a tube of NEB 5-alpha Competent E. coli cells on ice for 10 minutes.

Add 1-5 μl containing 1 pg-100 ng of plasmid DNA to the cell mixture. Carefully flick the tube 4-5 times to mix cells and DNA. Do not vortex.

Place the mixture on ice for 30 minutes. Do not mix.

Heat shock at 42° C. for exactly 30 seconds. Do not mix.

Place on ice for 5 minutes. Do not mix.

Pipette 950 μl of room temperature SOC into the mixture.

Place at 37° C. for 60 minutes. Shake vigorously (250 rpm) or rotate.

Warm selection plates to 37° C.

Mix the cells thoroughly by flicking the tube and inverting.

Spread 50-100 μl of each dilution onto a selection plate and incubate overnight at 37° C. Alternatively, incubate at 30° C. for 24-36 hours or 25° C. for 48 hours.

A single colony is then used to inoculate 5 ml of LB growth media using the appropriate antibiotic and then allowed to grow (250 RPM, 37° C.) for 5 hours. This is then used to inoculate a 200 ml culture medium and allowed to grow overnight under the same conditions.

To isolate the plasmid (up to 850 μg), a maxi prep is performed using the Invitrogen PURELINK™ HiPure Maxiprep Kit (Carlsbad, Calif.), following the manufacturer's instructions.

In order to generate cDNA for In Vitro Transcription (IVT), the plasmid is first linearized using a restriction enzyme such as XbaI. A typical restriction digest with XbaI will include the following: Plasmid 1.0 μg; 10× Buffer 1.0 μl; XbaI 1.5 μl; dH₂0 up to 10 μl; incubated at 37° C. for 1 hr. If performing at lab scale (<5 μg), the reaction is cleaned up using Invitrogen's PURELINK™ PCR Micro Kit (Carlsbad, Calif.) per manufacturer's instructions. Larger scale purifications may need to be done with a product that has a larger load capacity such as Invitrogen's standard PURELINK™ PCR Kit (Carlsbad, Calif.). Following the cleanup, the linearized vector is quantified using the NanoDrop and analyzed to confirm linearization using agarose gel electrophoresis.

### IVT Reaction

The in vitro transcription reaction generates mRNA containing alternative nucleotides or RNA. The input nucleotide triphosphate (NTP) mix is made in-house using natural and un-natural NTPs.

A typical in vitro transcription reaction includes the following:

| | |
|---|---|
| Template cDNA | 1.0 μg |
| 10x transcription buffer (400 mM Tris-HCl | 2.0 μl |
| pH 8.0, 190 mM MgCl2, 50 mM DTT, | |
| 10 mM Spermidine) | |
| Custom NTPs (25 mM each | 7.2 μl |
| RNase Inhibitor | 20 U |
| T7 RNA polymerase | 3000 U |
| dH₂0 | up to 20.0 μl |

Incubation at 37° C. for 3 hr-5 hrs.

The crude IVT mix may be stored at 4° C. overnight for cleanup the next day. 1 U of RNase-free DNase is then used to digest the original template. After 15 minutes of incubation at 37° C., the mRNA is purified using Ambion's MEGACLEAR™ Kit (Austin, Tex.) following the manufacturer's instructions. This kit can purify up to 500 μg of RNA. Following the cleanup, the RNA is quantified using the NanoDrop and analyzed by agarose gel electrophoresis to confirm the RNA is the proper size and that no degradation of the RNA has occurred.

The T7 RNA polymerase may be selected from, T7 RNA polymerase, T3 RNA polymerase and mutant polymerases such as, but not limited to, the novel polymerases able to incorporate alternative NTPs as well as those polymerases described by Liu (Esvelt et al. (Nature (2011) 472(7344):499-503 and U.S. Publication No. 20110177495) which recognize alternate promoters, Ellington (Chelliserrykattil and Ellington, Nature Biotechnology (2004) 22(9):1155-1160) describing a T7 RNA polymerase variant to transcribe 2′-O-methyl RNA and Sousa (Padilla and Sousa, Nucleic Acids Research (2002) 30(24): e128) describing a T7 RNA polymerase double mutant; herein incorporated by reference in their entireties.

B. Agarose Gel Electrophoresis of mRNA

Individual mRNAs (200-400 ng in a 20 μl volume) are loaded into a well on a non-denaturing 1.2% Agarose E-Gel (Invitrogen, Carlsbad, Calif.) and run for 12-15 minutes according to the manufacturer protocol.

C. Agarose Gel Electrophoresis of RT-PCR Products

Individual reverse transcribed-PCR products (200-400 ng) are loaded into a well of a non-denaturing 1.2% Agarose E-Gel (Invitrogen, Carlsbad, Calif.) and run for 12-15 minutes according to the manufacturer protocol.

D. Nanodrop mRNA Quantification and UV Spectral Data

MRNAs in TE buffer (1 μl) are used for Nanodrop UV absorbance readings to quantitate the yield of each mRNA from an in vitro transcription reaction (UV absorbance traces are not shown).

### Example 3. Enzymatic Capping of mRNA

Capping of the mRNA is performed as follows where the mixture includes: IVT RNA 60 μg-180 μg and dH₂0 up to 72 μl. The mixture is incubated at 65° C. for 5 minutes to denature RNA, and then is transferred immediately to ice.

The protocol then involves the mixing of 10× Capping Buffer (0.5 M Tris-HCl (pH 8.0), 60 mM KCl, 12.5 mM MgCl₂) (10.0 μl); 20 mM GTP (5.0 μl); 20 mM S-Adenosyl Methionine (2.5 μl); RNase Inhibitor (100 U); 2′-O-Methyltransferase (400U); Vaccinia capping enzyme (Guanylyl transferase) (40 U); dH₂0 (Up to 28 μl); and incubation at 37° C. for 30 minutes for 60 μg RNA or up to 2 hours for 180 μg of RNA.

The mRNA is then purified using Ambion's MEGACLEAR™ Kit (Austin, Tex.) following the manufacturer's instructions. Following the cleanup, the RNA is quantified using the NANODROP™ (ThermoFisher, Waltham, Mass.) and analyzed by agarose gel electrophoresis to confirm the RNA is the proper size and that no degradation of the RNA has occurred. The RNA product may also be sequenced by running a reverse-transcription-PCR to generate the cDNA for sequencing.

### Example 4. 5′-Guanosine Capping

A. Materials and Methods

The cloning, gene synthesis and vector sequencing may be performed by DNA2.0 Inc. (Menlo Park, Calif.). The ORF is restriction digested using XbaI and used for cDNA synthesis using tailed- or tail-less-PCR. The tailed-PCR cDNA product is used as the template for the mRNA synthesis reaction using 25 mM each alternative nucleotide mix (all alternative nucleotides may be custom synthesized or purchased from TriLink Biotech, San Diego, Calif. except pyrrolo-C triphosphate which may be purchased from Glen Research, Sterling Va.; unmodifed nucleotides are purchased from Epicenter Biotechnologies, Madison, Wis.) and CellScript MEGASCRIPT™ (Epicenter Biotechnologies, Madison, Wis.) complete mRNA synthesis kit.

The in vitro transcription reaction is run for 4 hours at 37° C. MRNAs incorporating adenosine analogs are poly (A) tailed using yeast Poly (A) Polymerase (Affymetrix, Santa Clara, Calif.). The PCR reaction uses HiFi PCR 2× MASTER MIX™ (Kapa Biosystems, Woburn, Mass.). MRNAs are post-transcriptionally capped using recombinant Vaccinia Virus Capping Enzyme (New England BioLabs, Ipswich, Mass.) and a recombinant 2′-O-methyltransferase (Epicenter Biotechnologies, Madison, Wis.) to generate the 5′-guanosine Cap1 structure. Cap 2 structure and Cap 2 structures may be generated using additional 2′-O-methyltransferases. The In vitro transcribed mRNA product is run on an agarose gel and visualized. MRNA may be purified with Ambion/Applied Biosystems (Austin, Tex.) MEGAClear RNA™ purification kit. The PCR uses PURELINK™ PCR purification kit (Invitrogen, Carlsbad, Calif.). The product is quantified on NANODROP™ UV Absorbance (ThermoFisher, Waltham, Mass.). Quality, UV absorbance quality and visualization of the product was performed on an 1.2% agarose gel. The product is resuspended in TE buffer.

B. 5′-Capping

5′-capping of mRNA may be completed concomitantly during the in vitro-transcription reaction using the following chemical RNA cap analogs to generate the 5′-guanosine cap structure according to manufacturer protocols: 3′-O-Me-m⁷G(5′)ppp(5′)G (the ARCA cap); G(5′)ppp(5′)A; G(5′)ppp(5′)G; m⁷G(5′)ppp(5′)A; m⁷G(5′)ppp(5′)G (New England BioLabs, Ipswich, Mass.). 5′-capping of mRNA may be completed post-transcriptionally using a Vaccinia Virus Capping Enzyme to generate the “Cap 0” structure: m⁷G(5′)ppp(5′)G (New England BioLabs, Ipswich, Mass.). Cap 1 structure may be generated using both Vaccinia Virus Capping Enzyme and a 2′-O methyl-transferase to generate: m7G(5′)ppp(5′)G-2′-O-methyl. Cap 2 structure may be generated from the Cap 1 structure followed by the 2′-o-methylation of the 5′-antepenultimate nucleotide using a 2′-0 methyl-transferase. Cap 3 structure may be generated from the Cap 2 structure followed by the 2′-o-methylation of the 5′-preantepenultimate nucleotide using a 2′-0 methyl-transferase. Enzymes are preferably derived from a recombinant source.

When transfected into mammalian cells, the mRNAs have a stability of 12-18 hours or more than 18 hours, e.g., 24, 36, 48, 60, 72 or greater than 72 hours.

### Example 5. PolyA Tailing Reaction

Without a poly-T in the cDNA, a poly-A tailing reaction must be performed before cleaning the final product. This is done by mixing Capped IVT RNA (100 μl); RNase Inhibitor (20 U); 10× Tailing Buffer (0.5 M Tris-HCl (pH 8.0), 2.5 M NaCl, 100 mM MgCl₂)(12.0 μl); 20 mM ATP (6.0 μl); Poly-A Polymerase (20 U); dH₂0 up to 123.5 μl and incubation at 37° C. for 30 min. If the poly-A tail is already in the transcript, then the tailing reaction may be skipped and proceed directly to cleanup with Ambion's MEGACLEAR™ kit (Austin, Tex.) (up to 500 μg). Poly-A Polymerase is preferably a recombinant enzyme expressed in yeast.

For studies performed and described herein, the poly-A tail is encoded in the IVT template to include 160 nucleotides in length. However, it should be understood that the processivity or integrity of the poly-A tailing reaction may not always result in exactly 160 nucleotides. Hence poly-A tails of approximately 160 nucleotides (SEQ ID NO: 9), and about 150-165 (SEQ ID NO: 3), 155 (SEQ ID NO: 4), 156 (SEQ ID NO: 5), 157 (SEQ ID NO: 6), 158 (SEQ ID NO: 7), 159 (SEQ ID NO: 8), 160 (SEQ ID NO: 9), 161 (SEQ ID NO: 10), 162 (SEQ ID NO: 11), 163 (SEQ ID NO: 12), 164 (SEQ ID NO: 13) or 165 (SEQ ID NO: 14) are within the scope of the invention.

### Example 6. Method of Screening for Protein Expression

A. Electrospray Ionization

A biological sample which may contain proteins encoded by RNA administered to the subject is prepared and analyzed according to the manufacturer protocol for electrospray ionization (ESI) using 1, 2, 3 or 4 mass analyzers. A biologic sample may also be analyzed using a tandem ESI mass spectrometry system.

Patterns of protein fragments, or whole proteins, are compared to known controls for a given protein and identity is determined by comparison.

B. Matrix-Assisted Laser Desorption/Ionization

A biological sample which may contain proteins encoded by RNA administered to the subject is prepared and analyzed according to the manufacturer protocol for matrix-assisted laser desorption/ionization (MALDI).

Patterns of protein fragments, or whole proteins, are compared to known controls for a given protein and identity is determined by comparison.

C. Liquid Chromatography-Mass Spectrometry-Mass Spectrometry

A biological sample, which may contain proteins encoded by RNA, may be treated with a trypsin enzyme to digest the proteins contained within. The resulting peptides are analyzed by liquid chromatography-mass spectrometry-mass spectrometry (LC/MS/MS). The peptides are fragmented in the mass spectrometer to yield diagnostic patterns that can be matched to protein sequence databases via computer algorithms. The digested sample may be diluted to achieve 1 ng or less starting material for a given protein. Biological samples containing a simple buffer background (e.g. water or volatile salts) are amenable to direct in-solution digest; more complex backgrounds (e.g. detergent, non-volatile salts, glycerol) require an additional clean-up step to facilitate the sample analysis.

Patterns of protein fragments, or whole proteins, are compared to known controls for a given protein and identity is determined by comparison.

### Example 7. Transfection

A. Reverse Transfection

For experiments performed in a 24-well collagen-coated tissue culture plate, Keratinocytes or other cells are seeded at a cell density of 1×10⁵. For experiments performed in a 96-well collagen-coated tissue culture plate, Keratinocytes are seeded at a cell density of 0.5×10⁵. For each mRNA to be transfected, mRNA: RNAIMAX™ are prepared as described and mixed with the cells in the multi-well plate within 6 hours of cell seeding before cells had adhered to the tissue culture plate.

B. Forward Transfection

In a 24-well collagen-coated tissue culture plate, Cells are seeded at a cell density of 0.7×10⁵. For experiments performed in a 96-well collagen-coated tissue culture plate, Keratinocytes, if used, are seeded at a cell density of 0.3×10⁵. Cells are then grown to a confluency of >70% for over 24 hours. For each mRNA to be transfected, mRNA: RNAIMAX™ are prepared as described and transfected onto the cells in the multi-well plate over 24 hours after cell seeding and adherence to the tissue culture plate.

C. Translation Screen: ELISA

Cells are grown in EpiLife medium with Supplement S7 from Invitrogen at a confluence of >70%. Cells are reverse transfected with 300 ng of the indicated chemically mRNA complexed with RNAIMAX™ from Invitrogen. Alternatively, cells are forward transfected with 300 ng mRNA complexed with RNAIMAX™ from Invitrogen. The RNA: RNAIMAX™ complex is formed by first incubating the RNA with Supplement-free EPILIFE® media in a 5× volumetric dilution for 10 minutes at room temperature.

In a second vial, RNAIMAX™ reagent is incubated with Supplement-free EPILIFE® Media in a 10× volumetric dilution for 10 minutes at room temperature. The RNA vial is then mixed with the RNAIMAX™ vial and incubated for 20-30 at room temperature before being added to the cells in a drop-wise fashion. Secreted polypeptide concentration in the culture medium is measured at 18 hours post-transfection for each of the mRNAs in triplicate. Secretion of the polypeptide of interest from transfected human cells is quantified using an ELISA kit from Invitrogen or R&D Systems (Minneapolis, Minn.) following the manufacturers recommended instructions.

D. Dose and Duration: ELISA

Cells are grown in EPILIFE® medium with Supplement S7 from Invitrogen at a confluence of >70%. Cells are reverse transfected with Ong, 46.875 ng, 93.75 ng, 187.5 ng, 375 ng, 750 ng, or 1500 ng mRNA complexed with RNAIMAX™ from Invitrogen. The mRNA: RNAIMAX™ complex is formed as described. Secreted polypeptide concentration in the culture medium is measured at 0, 6, 12, 24, and 48 hours post-transfection for each concentration of each mRNA in triplicate. Secretion of the polypeptide of interest from transfected human cells is quantified using an ELISA kit from Invitrogen or R&D Systems following the manufacturers recommended instructions.

### Example 8. Cellular Innate Immune Response: IFN-Beta ELISA and TNF-Alpha ELISA

An enzyme-linked immunosorbent assay (ELISA) for Human Tumor Necrosis Factor-α (TNF-α), Human Interferon-β (IFN-β) and Human Granulocyte-Colony Stimulating Factor (G-CSF) secreted from in vitro-transfected Human Keratinocyte cells is tested for the detection of a cellular innate immune response.

Cells are grown in EPILIFE® medium with Human Growth Supplement in the absence of hydrocortisone from Invitrogen at a confluence of >70%. Cells are reverse transfected with Ong, 93.75 ng, 187.5 ng, 375 ng, 750 ng, 1500 ng or 3000 ng of the indicated mRNA complexed with RNAIMAX™ from Invitrogen as described in triplicate. Secreted TNF-α in the culture medium is measured 24 hours post-transfection for each of the mRNAs using an ELISA kit from Invitrogen according to the manufacturer protocols.

Secreted IFN-β is measured 24 hours post-transfection for each of the mRNAs using an ELISA kit from Invitrogen according to the manufacturer protocols. Secreted hu-G-CSF concentration is measured at 24 hours post-transfection for each of the mRNAs. Secretion of the polypeptide of interest from transfected human cells is quantified using an ELISA kit from Invitrogen or R&D Systems (Minneapolis, Minn.) following the manufacturers recommended instructions. These data indicate which mRNA are capable eliciting a reduced cellular innate immune response in comparison to natural and other alternative polynucleotides or reference compounds by measuring exemplary type 1 cytokines such as TNF-alpha and IFN-beta.

### Example 9. Cytotoxicity and Apoptosis

This experiment demonstrates cellular viability, cytotoxity and apoptosis for distinct mRNA-in vitro transfected Human Keratinocyte cells. Keratinocytes are grown in EPILIFE® medium with Human Keratinocyte Growth Supplement in the absence of hydrocortisone from Invitrogen at a confluence of >70%. Keratinocytes are reverse transfected with Ong, 46.875 ng, 93.75 ng, 187.5 ng, 375 ng, 750 ng, 1500 ng, 3000 ng, or 6000 ng of mRNA complexed with RNAIMAX™ from Invitrogen. The mRNA: RNAIMAX™ complex is formed. Secreted huG-CSF concentration in the culture medium is measured at 0, 6, 12, 24, and 48 hours post-transfection for each concentration of each mRNA in triplicate. Secretion of the polypeptide of interest from transfected human keratinocytes is quantified using an ELISA kit from Invitrogen or R&D Systems following the manufacturers recommended instructions. Cellular viability, cytotoxicity and apoptosis is measured at 0, 12, 48, 96, and 192 hours post-transfection using the APOTOX-GLO™ kit from Promega (Madison, Wis.) according to manufacturer instructions.

### Example 10. Incorporation of Naturally and Alternatively Occurring Nucleosides

Naturally and alternatively occurring nucleosides are incorporated into mRNA encoding a polypeptide of interest. Certain commercially available nucleoside triphosphates (NTPs) are investigated in the polynucleotides of the invention. A selection of these is given in Table 5. The resultant mRNAs are then examined for their ability to produce protein, induce cytokines, and/or produce a therapeutic outcome.

TABLE 5
Naturally occurring nucleosides.

| | Naturally |
|---|---|
| Chemistry alteration | occurring |
| 2′-O-methylcytidine TP | Y |
| 4-thiouridine TP | Y |
| 2′-O-methyluridine TP | Y |
| 5-methyl-2-thiouridine TP | Y |
| 5,2′-O-dimethyluridine TP | Y |
| 5-aminomethyl-2-thiouridine TP | Y |
| 5,2′-O-dimethylcytidine TP | Y |
| 2-methylthio-N6-isopentenyladenosine TP | Y |
| 2′-O-methyladenosine TP | Y |
| 2′-O-methylguanosine TP | Y |
| N6-methyl-N6-threonylcarbamoyladenosine TP | Y |
| N6-hydroxynorvalylcarbamoyladenosine TP | Y |
| 2-methylthio-N6-hydroxynorvalyl carbamoyladenosine TP | Y |
| 2′-O-ribosyladenosine (phosphate) TP | Y |
| N6,2′-O-dimethyladenosine TP | Y |
| N6,N6,2′-O-trimethyladenosine TP | Y |
| 1,2′-O-dimethyladenosine TP | Y |
| N6-acetyladenosine TP | Y |
| 2-methyladenosine TP | Y |
| 2-methylthio-N6-methyladenosine TP | Y |
| N2,2′-O-dimethylguanosine TP | Y |
| N2,N2,2′-O-trimethylguanosine TP | Y |
| 7-cyano-7-deazaguanosine TP | Y |
| 7-aminomethyl-7-deazaguanosine TP | Y |
| 2′-O-ribosylguanosine (phosphate) TP | Y |
| N2,7-dimethylguanosine TP | Y |
| N2,N2,7-trimethylguanosine TP | Y |
| 1,2′-O-dimethylguanosine TP | Y |
| Peroxywybutosine TP | Y |
| Hydroxywybutosine TP | Y |
| undermodified hydroxywybutosine TP | Y |
| Methylwyosine TP | Y |
| N2,7,2′-O-trimethylguanosine TP | Y |
| 1,2′-O-dimethylinosine TP | Y |
| 2′-O-methylinosine TP | Y |
| 4-demethylwyosine TP | Y |
| Isowyosine TP | Y |
| Queuosine TP | Y |
| Epoxyqueuosine TP | Y |
| galactosyl-queuosine TP | Y |
| mannosyl-queuosine TP | Y |
| Archaeosine TP | Y |

Alternative nucleotides of the present invention may also include those listed below in Table 6.

TABLE 6
Alternatively occurring nucleotides.

| | Naturally |
|---|---|
| Chemistry alteration | occurring |
| 5-(1-Propynyl)ara-uridine TP | N |
| 2′-O-Methyl-5-(1-propynyl)uridine TP | N |
| 2′-O-Methyl-5-(1-propynyl)cytidine TP | N |
| 5-(1-Propynyl)ara-cytidine TP | N |
| 5-Ethynylara-cytidine TP | N |
| 5-Ethynylcytidine TP | N |
| 5-Vinylarauridine TP | N |
| (Z)-5-(2-Bromo-vinyl)ara-uridine TP | N |
| (E)-5-(2-Bromo-vinyl)ara-uridine TP | N |
| (Z)-5-(2-Bromo-vinyl)uridine TP | N |
| (E)-5-(2-Bromo-vinyl)uridine TP | N |
| 5-Methoxycytidine TP | N |
| 5-Formyluridine TP | N |
| 5-Cyanouridine TP | N |
| 5-Dimethylaminouridine TP | N |
| 5-Trideuteromethyl-6-deuterouridine TP | N |
| 5-Cyanocytidine TP | N |
| 5-(2-Chloro-phenyl)-2-thiocytidine TP | N |
| 5-(4-Amino-phenyl)-2-thiocytidine TP | N |
| 5-(2-Furanyl)uridine TP | N |
| 5-Phenylethynyluridine TP | N |
| N4,2′-O-Dimethylcytidine TP | N |
| 3′-Ethynylcytidine TP | N |
| 4′-Carbocyclic adenosine TP | N |
| 4′-Carbocyclic cytidine TP | N |
| 4′-Carbocyclic guanosine TP | N |
| 4′-Carbocyclic uridine TP | N |
| 4′-Ethynyladenosine TP | N |
| 4′-Ethynyluridine TP | N |
| 4′-Ethynylcytidine TP | N |
| 4′-Ethynylguanosine TP | N |
| 4′-Azidouridine TP | N |
| 4′-Azidocytidine TP | N |
| 4′-Azidoadenosine TP | N |
| 4′-Azidoguanosine TP | N |
| 2′-Deoxy-2′,2′-difluorocytidine TP | N |
| 2′-Deoxy-2′,2′-difluorouridine TP | N |
| 2′-Deoxy-2′,2′-difluoroadenosine TP | N |
| 2′-Deoxy-2′,2′-difluoroguanosine TP | N |
| 2′-Deoxy-2′-b-fluorocytidine TP | N |
| 2′-Deoxy-2′-b-fluorouridine TP | N |
| 2′-Deoxy-2′-b-fluoroadenosine TP | N |
| 2′-Deoxy-2′-b-fluoroguanosine TP | N |
| 8-Trifluoromethyladenosine TP | N |
| 2′-Deoxy-2′-b-chlorouridine TP | N |
| 2′-Deoxy-2′-b-bromouridine TP | N |
| 2′-Deoxy-2′-b-iodouridine TP | N |
| 2′-Deoxy-2′-b-chlorocytidine TP | N |
| 2′-Deoxy-2′-b-bromocytidine TP | N |
| 2′-Deoxy-2′-b-iodocytidine TP | N |
| 2′-Deoxy-2′-b-chloroadenosine TP | N |
| 2′-Deoxy-2′-b-bromoadenosine TP | N |
| 2′-Deoxy-2′-b-iodoadenosine TP | N |
| 2′-Deoxy-2′-b-chloroguanosine TP | N |
| 2′-Deoxy-2′-b-bromoguanosine TP | N |
| 2′-Deoxy-2′-b-iodoguanosine TP | N |
| 5′-Homo-cytidine TP | N |
| 5′-Homo-adenosine TP | N |
| 5′-Homo-uridine TP | N |
| 5′-Homo-guanosine TP | N |
| 2′-Deoxy-2′-a-mercaptouridine TP | N |
| 2′-Deoxy-2′-a-thiomethoxyuridine TP | N |
| 2′-Deoxy-2′-a-azidouridine TP | N |
| 2′-Deoxy-2′-a-aminouridine TP | N |
| 2′-Deoxy-2′-a-mercaptocytidine TP | N |
| 2′-Deoxy-2′-a-thiomethoxycytidine TP | N |
| 2′-Deoxy-2′-a-azidocytidine TP | N |
| 2′-Deoxy-2′-a-aminocytidine TP | N |
| 2′-Deoxy-2′-a-mercaptoadenosine TP | N |
| 2′-Deoxy-2′-a-thiomethoxyadenosine TP | N |
| 2′-Deoxy-2′-a-azidoadenosine TP | N |
| 2′-Deoxy-2′-a-aminoadenosine TP | N |
| 2′-Deoxy-2′-a-mercaptoguanosine TP | N |
| 2′-Deoxy-2′-a-thiomethoxyguanosine TP | N |
| 2′-Deoxy-2′-a-azidoguanosine TP | N |
| 2′-Deoxy-2′-a-aminoguanosine TP | N |
| 2′-Deoxy-2′-b-mercaptouridine TP | N |
| 2′-Deoxy-2′-b-thiomethoxyuridine TP | N |
| 2′-Deoxy-2′-b-azidouridine TP | N |
| 2′-Deoxy-2′-b-aminouridine TP | N |
| 2′-Deoxy-2′-b-mercaptocytidine TP | N |
| 2′-Deoxy-2′-b-thiomethoxycytidine TP | N |
| 2′-Deoxy-2′-b-azidocytidine TP | N |
| 2′-Deoxy-2′-b-aminocytidine TP | N |
| 2′-Deoxy-2′-b-mercaptoadenosine TP | N |
| 2′-Deoxy-2′-b-thiomethoxyadenosine TP | N |
| 2′-Deoxy-2′-b-azidoadenosine TP | N |
| 2′-Deoxy-2′-b-aminoadenosine TP | N |
| 2′-Deoxy-2′-b-mercaptoguanosine TP | N |
| 2′-Deoxy-2′-b-thiomethoxyguanosine TP | N |
| 2′-Deoxy-2′-b-azidoguanosine TP | N |
| 2′-Deoxy-2′-b-aminoguanosine TP | N |
| 2′-b-Trifluoromethyladenosine TP | N |
| 2′-b-Trifluoromethylcytidine TP | N |
| 2′-b-Trifluoromethylguanosine TP | N |
| 2′-b-Trifluoromethyluridine TP | N |
| 2′-a-Trifluoromethyladenosine TP | N |
| 2′-a-Trifluoromethylcytidine TP | N |
| 2′-a-Trifluoromethylguanosine TP | N |
| 2′-a-Trifluoromethyluridine TP | N |
| 2′-b-Ethynyladenosine TP | N |
| 2′-b-Ethynylcytidine TP | N |
| 2′-b-Ethynylguanosine TP | N |
| 2′-b-Ethynyluridine TP | N |
| 2′-a-Ethynyladenosine TP | N |
| 2′-a-Ethynylcytidine TP | N |
| 2′-a-Ethynylguanosine TP | N |
| 2′-a-Ethynyluridine TP | N |
| (E)-5-(2-Bromo-vinyl)cytidine TP | N |
| 2-Trifluoromethyladenosine TP | N |
| 2-Mercaptoadenosine TP | N |
| 2-Aminoadenosine TP | N |
| 2-Azidoadenosine TP | N |
| 2-Fluoroadenosine TP | N |
| 2-Chloroadenosine TP | N |
| 2-Bromoadenosine TP | N |
| 2-Iodoadenosine TP | N |
| Formycin A TP | N |
| Formycin B TP | N |
| Oxoformycin TP | N |
| Pyrrolosine TP | N |
| 9-Deazaadenosine TP | N |
| 9-Deazaguanosine TP | N |
| 3-Deazaadenosine TP | N |
| 3-Deaza-3-fluoroadenosine TP | N |
| 3-Deaza-3-chloroadenosine TP | N |
| 3-Deaza-3-bromoadenosine TP | N |
| 3-Deaza-3-iodoadenosine TP | N |
| 1-Deazaadenosine TP | N |

### Example 11. Association of Oligonucleotides with mRNA

Association of Oligonucleotides with mRNA

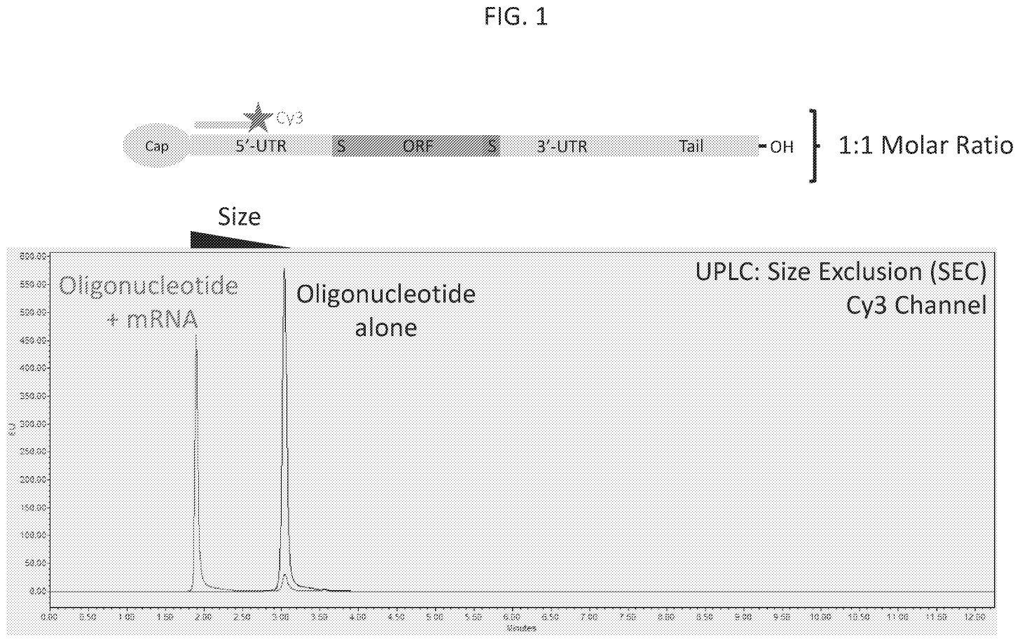

Compositions including an mRNA and one of more oligonucleotides (e.g., one or more conjugates including an oligonucleotide covalently conjugated to one or more moieties) were evaluated for the ability of the oligonucleotide to associate with (e.g., hybridize with) an mRNA. A 20 nucleotide oligonucleotide covalently conjugated to a Cy3 dye was annealed to an mRNA in a 1:1 ratio. Size exclusion chromatography was used to evaluate the level of association, which is shown in FIG. 1.

Association of Oligonucleotides Conjugated to Bulky Moieties with mRNA

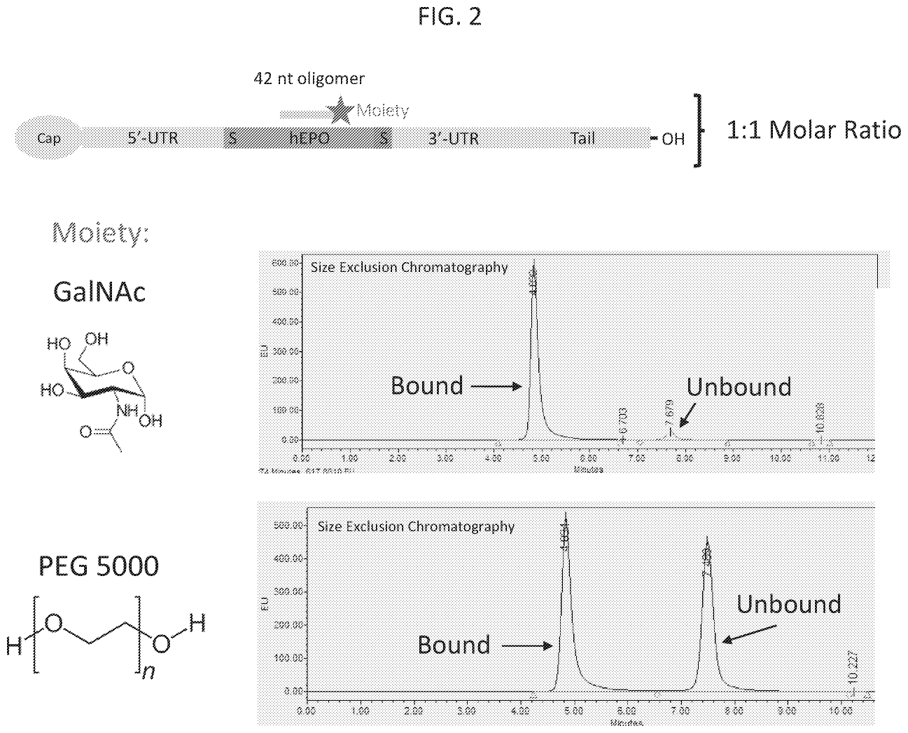

The ability of an oligonucleotide conjugated to sterically bulky moiety was determined. A 20 nucleotide oligonucleotide covalently conjugated to either a sugar moiety (e.g., GalNac) or a polyethylene glycol (e.g., PEG 5000) was annealed to mRNA in a 1:1 ratio. Size exclusion chromatography was used to evaluate the level of association, which is shown in FIG. 2.

Requirement for Sequence Complementarity for Association

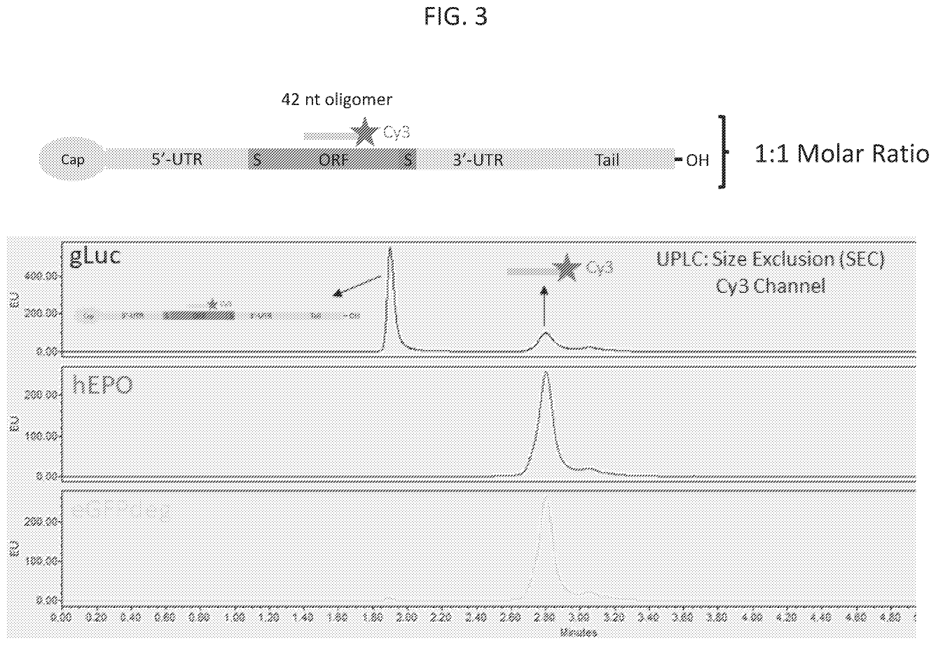

The requirement for sequence complementarity was also determined. A 42 nucleotide oligonucleotide complementary to a region of nucleotides in the open reading frame of a Gaussia Luciferase (gLuc) mRNA was conjugated to Cy3. The conjugate was evaluated for its ability to bind either gLuc mRNA, human erythropoietin (hEPO) mRNA, or green fluorescence protein (eGFPdeg) mRNA. The conjugate was annealed to the mRNA in a 1:1 ratio and the level of binding was determined by size exclusion chromatography. The conjugate was determined to associate with gLuc, but not with hEPO or eGFPdeg mRNA. Results are provided in FIG. 3.

Length and Location Dependence for Association

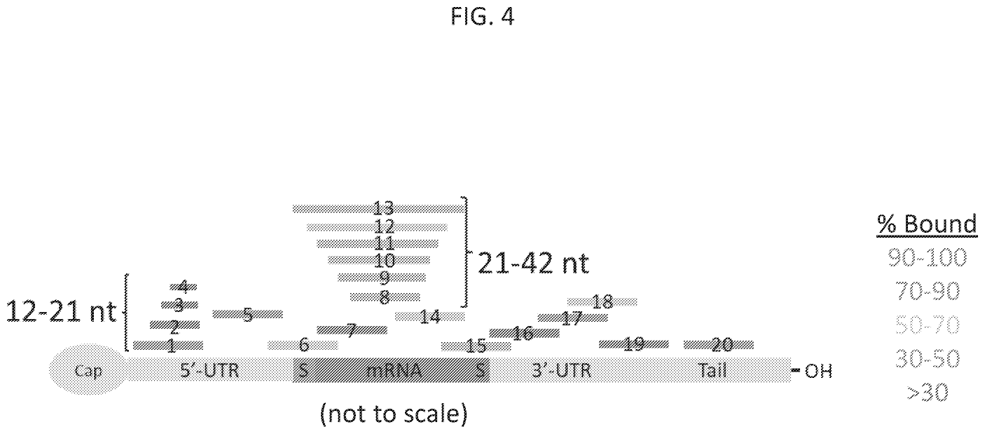

Twenty oligonucleotide-Cy3 conjugates having different lengths (12-42 nucleotides) and locations of sequence complementarity to an mRNA were evaluated for their ability to bind an mRNA. As shown in FIG. 4, association appeared to be sequence-specific, but the evaluated changes in oligonucleotide length did not alter binding in a significant manner.

Method: Annealing

Annealing of oligonucleotides or conjugates and mRNA was performed in buffer containing 25 mM potassium chloride (Ambion, Waltham, Mass.) and 25 μM ethylenediaminetetraacetic acid (Ambion, Waltham, Mass.). Oligonucleotides or conjugates and mRNA were combined in the desired ratio with buffer and then heated to 70° C. before cooling at a rate of 1° C./second to a temperature of 25° C.

Method: Size-Exclusion Chromatography

Separations were run on a Waters HPLC (Waters, Milford, Mass.) with an isocratic method (100 mM Tris acetate/EDTA, pH8) at a flowrate of 0.2 mL/min at 25° C. using a Sepax Zenix-300 4.6×150 mM column (Sepax, Newark, Del.). Spectra were obtained using fluorescence detection with fluorophore-dependent excitations and emission wavelengths. Injection were performed at 10 μL scale at 0.1 mg/mL mRNA.

### Example 12. Determination of Location-Dependence for mRNA Expression

Twenty 2′-OMe oligonucleotide-Cy3 conjugates having different locations of sequence complementarity to a gLuc mRNA were annealed to the gLuc mRNA and evaluated for their effect on mRNA expression. Annealing was performed as described in Example 11 and quantification of expression of gLuc was performed as described in the IncuCyte Expression assay described below.

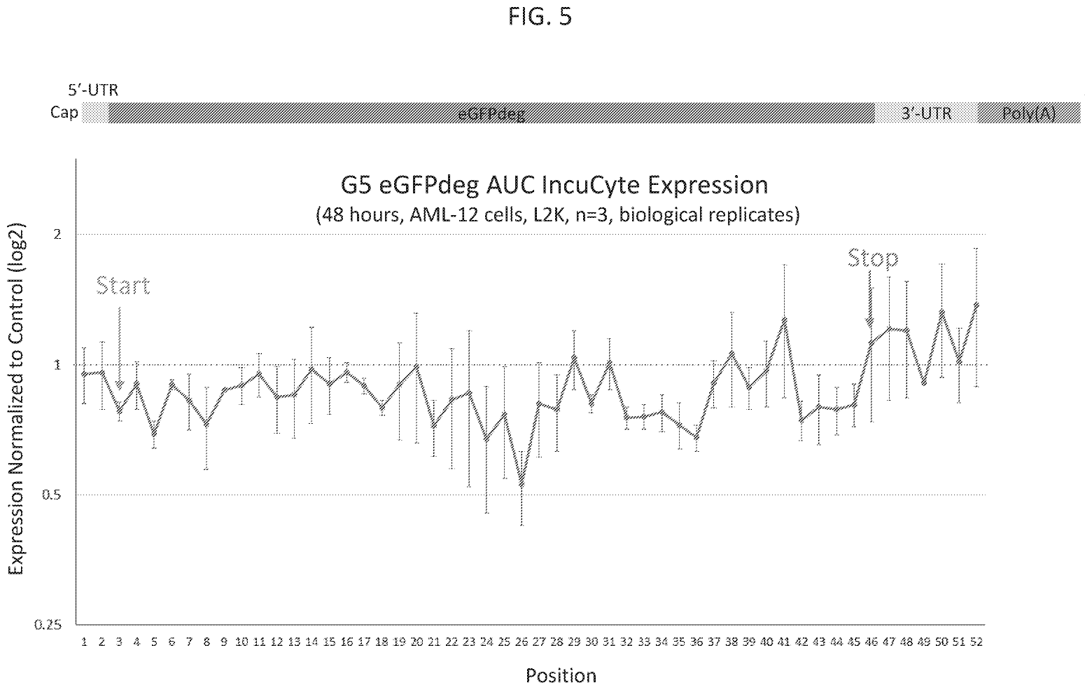

Like association, expression levels show some degree of location-dependence. Expression was observed with all conjugates tested. Annealing of certain conjugates (e.g., conjugates complementary to the portion of the mRNA containing the stop codon or the portion of the mRNA following the stop codon) showed higher expression than the mRNA alone. Expression data is provided in FIG. 5.

IncuCyte Expression of eGFPdeg

Cell plating for eGFPdeg expression assay: Cells were seeded into 96 well culture plate (Costar 3596-Corning, Corning, N.Y.) at a density of 8,000 cells per well. 100 μL of the cell seed was added to all interior wells of the plate, while 160 μL of blank media was added to edge wells. Cells are incubated over night at 37° C. with 5% CO₂ prior to transfection.

Lipofectamine Transfection for eGFPdeg expression assay: mRNA at a concentration of 50 ng/μL was added to OPTI-MEM 1× (gibco-Thermo Fisher Scientific, Waltham, Mass.) at a 1:9 volumetric ratio, resulting in a mRNA mixture. Lipofectamine 2000 Reagent (invitrogen-Thermo Fisher Scientific, Waltham, Mass.) was add to OPTI-MEM 1× (gibco-Thermo Fisher Scientific, Waltham, Mass.) at a 1:19 vol/vol ratio, resulting in a L2K mixture. An equivalent volume of L2K mixture was added to mRNA mixture [40 μL to 40 μL], the resulting L2K mRNA mixture was incubated at room temperature for 20 minutes. 20 μL of the final L2K mRNA mixture was added directly to interior wells of the cell plate, resulting in a final mRNA dose of 50 ng per well.

Incucyte setup for reoccurring fluorescence reads: Dosed cell plate was tilted north, south, east, and west to ensure distribution of L2K mRNA mixture. Dosed plate was loaded into Incucyte Zoom (Essen Bioscience, Ann Arbor, Mich.). Within IncuCyte Zoom 2018A software, a vessel was added to the virtual tray. Green fluorescence was selected, as well as a scan pattern and processing definition [based on the number of samples and cell type respectively]. A reoccurring read was scheduled for 48 hours. Kinetic eGFPdeg expression graphs are generated by Zoom 2018A software, and AUC (area under curve) data was processed in Excel 2016 (Microsoft, Albuquerque, N. Mex.).

### Example 13. Innate Immune Response of mRNA in Complex with One or More Oligonucleotides

Immune Response in Cells

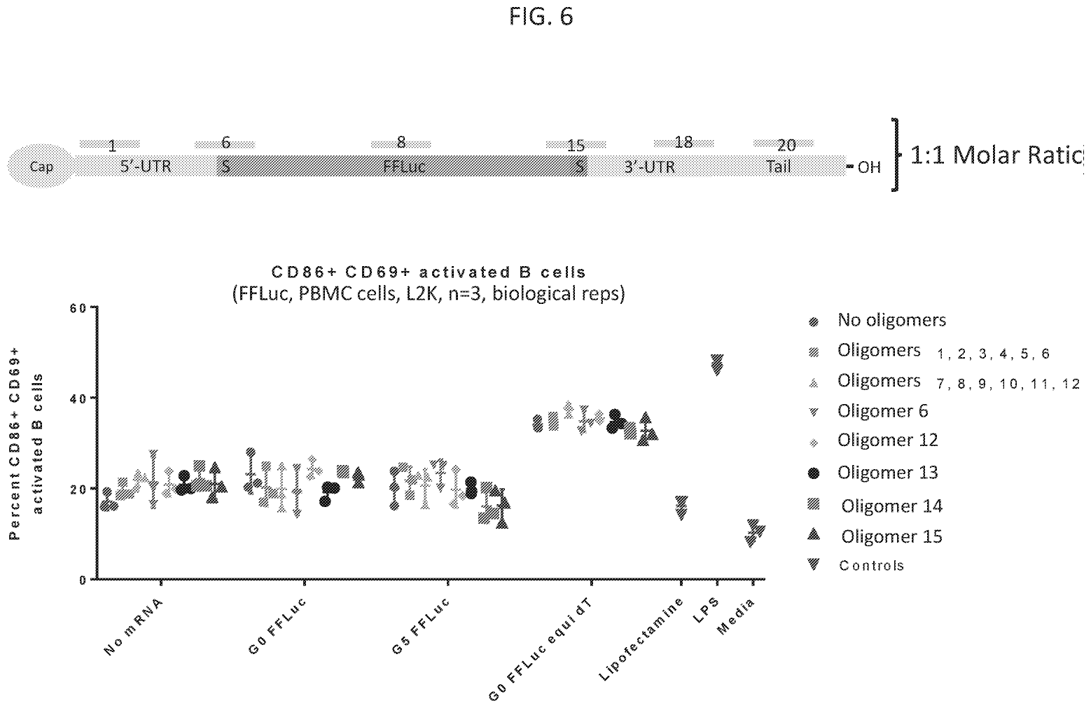

Compositions including an mRNA and an oligonucleotide complementary to the mRNA were evaluated for their ability to activate the innate immune response in cells (measured by B-cell activation), as compared to the mRNA alone. Oligonucleotides having sequence complementarity to different locations on a reporter mRNA (FFLuc) were evaluated. Oligonucleotides were annealed in a 1:1 ratio as described in Example 11. Methods for determining CD86+CD69+ B-cell activation are provided below, and the corresponding results are shown in FIG. 6. No substantial or significant change in innate immune response was detected under any oligonucleotide conditions compared to no-oligonucleotide controls.

Immune Response In Vivo

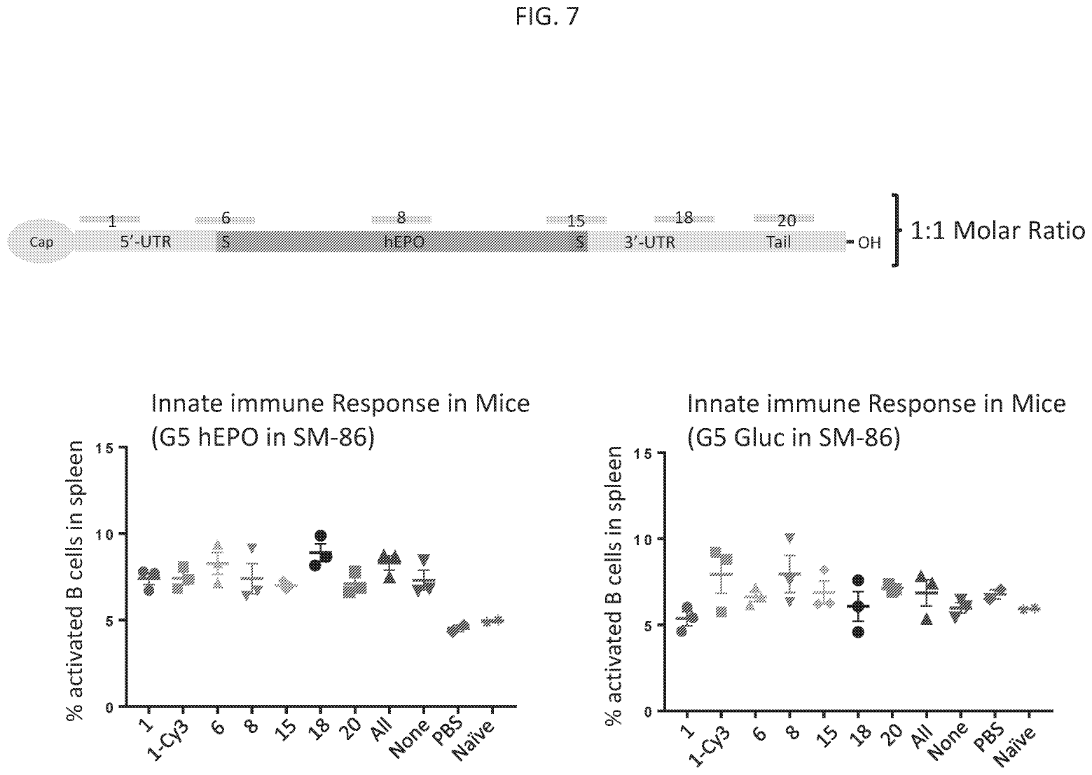

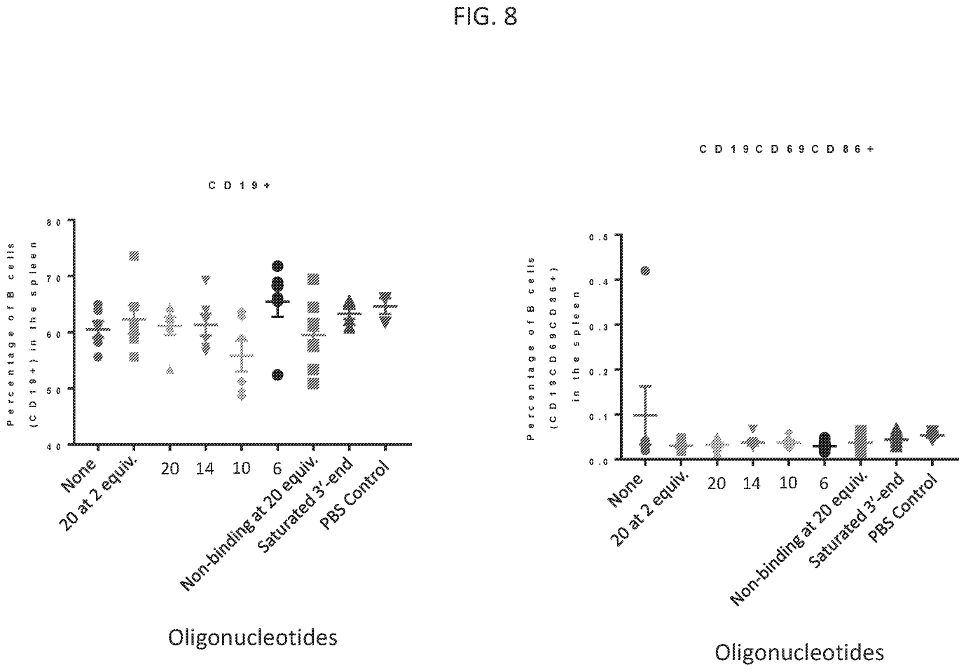

Compositions including an mRNA and an oligonucleotide complementary to the mRNA were evaluated for their ability to activate the innate immune response in mice (measured by B cells activation), as compared to the mRNA alone. Oligonucleotides having sequence complementarity to different locations on a reporter mRNA (either hEPO or gLuc) were evaluated. Oligonucleotides were annealed in a 1:1 ratio as described in Example 11. Methods for determining the percentage of B cell activation in the spleens of mice are provided below, and the corresponding results are shown in FIGS. 7-8. No substantial or significant change in innate immune response was detected under any oligonucleotide conditions compared to no-oligonucleotide controls.

Method: Determination of CD9+, CD19+CD86+, CD69+ B Cell Immune Response

PBMC cells (obtained from donor Leukopaks from StemCell, Vancouver, BC, Canada) were thawed in a water bath at 37° C. 40 mL of RPMI without FBS was transferred and spun down at 1500 RPM for 5 minutes at 4° C. Cells were resuspended in fresh media. 100 μl of splenocytes (200,000 cells) were added to each well of a 96-well flat-bottom plate. For each mRNA sample, 1 μg of sample was added to 25 μl of opti-MEM media with 5 ul lipofectamine 2000 (Thermo Fisher Scientific, Waltham, Mass.). Mixtures were incubated at room temperature for 5 minutes. 101 of each mixture was added on top of cells in the 96-well flat-bottom plate followed by the addition of 100 μl of complete media with mixing by pipette. Plates were incubated at 37° C. for 20 hours prior to staining. The contents of the wells were transferred to a fresh 96-well v-bottom plates and spun at 1500 RPM for 3 minutes at 4° C. Supernatant was removed and the cells were washed with 1× with FACS buffer (PBS pH 7.2+2% HI FBS). The spin was repeated and the supernatant was discarded. Pellets were resuspended in 100 μl antibody cocktail per well. The antibody cocktail contained CD19-APC, CD3-FITC, CD86-BV421, CD69-AF700 antibodies (at a 1:200 vol/vol ratio, Biolegend, San Diego, Calif.) and the stain proceeded for 20 minutes on ice. Cells were washed twice with FACS buffer and then resuspended in FACS buffer before analysis by flow cytometry.

Method: Determination of % Activated B Cells in Spleens of Mice

Spleens were removed and mechanically homogenized via passage through a 70-micron filter, then washed with PBS+2% fetal bovine serum. ACK lysis buffer (Gibco, catalogue #A10492-01) was used to lyse red blood cells. Cells were transferred to a 96-well plate for staining and washed with PBS+2% fetal bovine serum. Cells were stained for 20 minutes on ice with the following antibodies: anti-CD3 efluor450 (eBioscience, catalogue #48-0031-82), anti-CD19 Alexafluor 700 (Invitrogen, catalogue #56-0193-82), anti-CD69 APC (BioLegend, catalogue #104514), and anti-CD86 PEcy5 (Invitrogen, catalogue #15-0862-82). Cells were washed three times with PBS+2% fetal bovine serum, then analyzed on an LSR Fortessa flow cytometer.

### Example 14. Expression of mRNA In Vivo in Complex with One or More Oligonucleotides

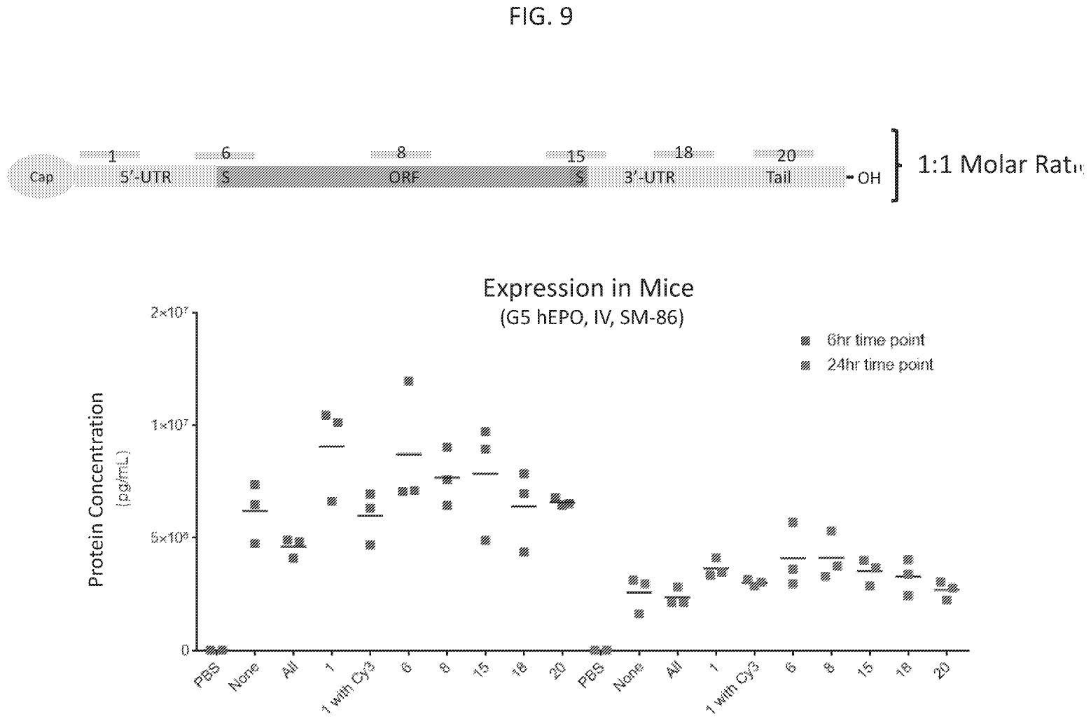

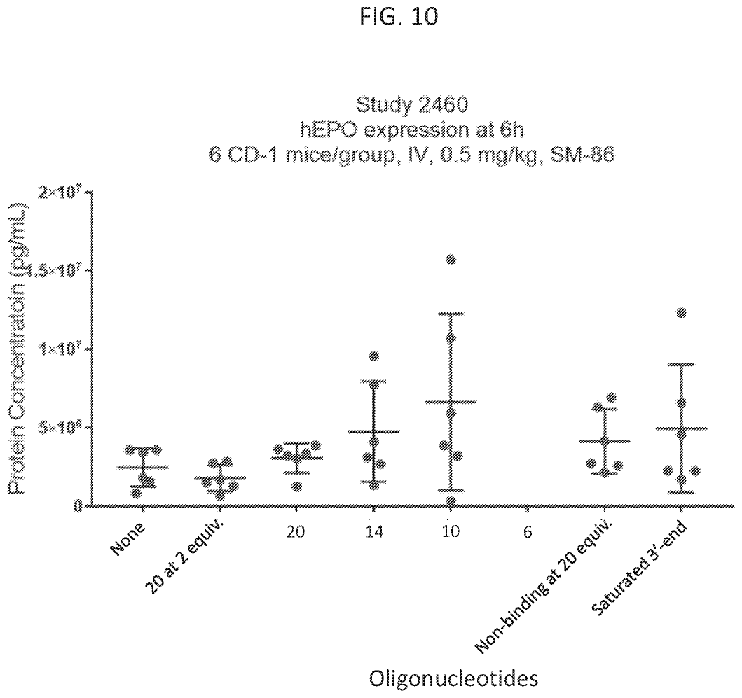

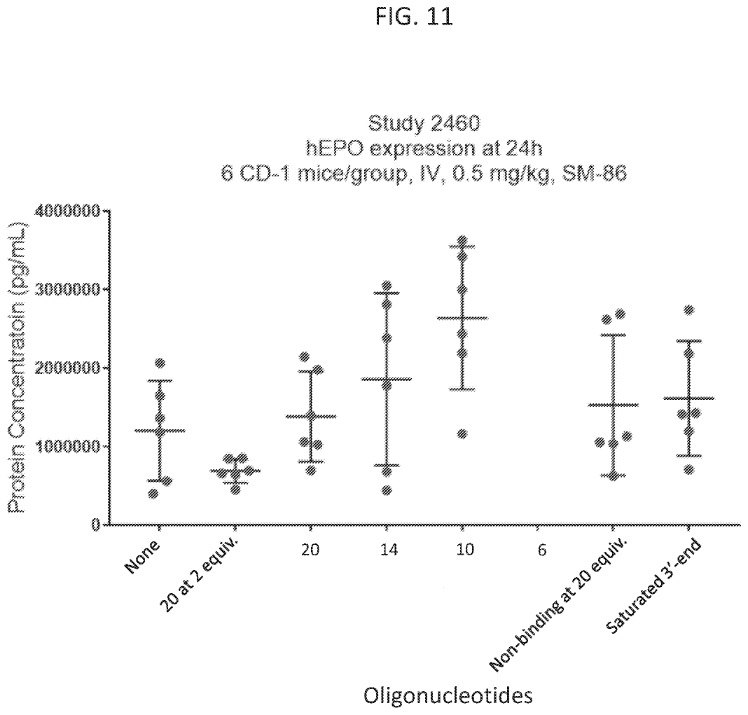

Compositions including an mRNA and an oligonucleotide complementary to the mRNA were evaluated for their ability to affect expression of the mRNA in mice, as compared to the mRNA alone. Oligonucleotides having sequence complementarity to different locations on a reporter mRNA (either hEPO) were evaluated. Oligonucleotides were annealed in a 1:1 ratio as described in Example 11. Either the composition including the mRNA and the complementary oligonucleotide or just the mRNA alone were administered intravenously to mice. The expression of the mRNA was quantified at 6 hours and 24 hours post-administration. Corresponding results are shown in FIGS. 9-11. No substantial or significant change in expression was detected under any oligonucleotide conditions compared to no-oligonucleotide controls.

### Example 15. Increased Serum Half-Life of mRNA in Complex with One or More Oligonucleotides

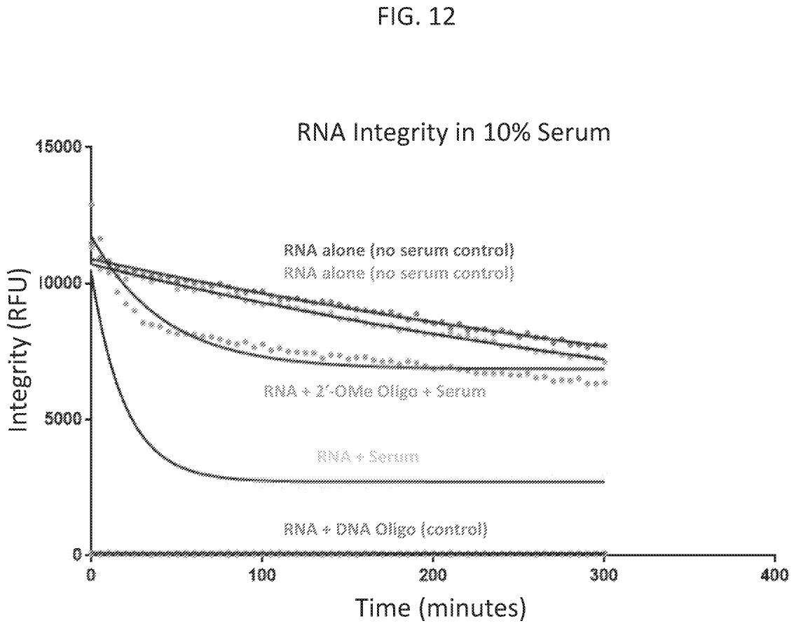

Compositions including an mRNA and an oligonucleotide complementary to the mRNA were evaluated for their ability to affect the serum half-life of an mRNA, as compared to the mRNA alone. A 2′-OMe oligonucleotides was annealed to an mRNA as described in Example 11. Serum half-life was determined as described below. The mRNA complexed with the 2′-OMe oligomer showed increased integrity relative to the mRNA alone. Corresponding results are shown in FIG. 12.

Method: Determination of Half-Life in Serum

RNA molecular beacons functionalized with Cy3 at the 5′-end and Black Hole Quencher at the 3′-end (Integrated RNA Technologies, Skokie, II) were annealed to oligonucleotides. Annealing was performed as described in Example 11. These mixtures were then combined with 10% human serum from human male AB plasma, USA origin, sterile-filtered (Sigma-Aldrich, St. Louis, Mo.) and left to incubate at room temperature. Molecular beacon integrity was monitored over time by fluorescence with excitation and emission at 550 nm and 600 nm, respectively.

### Example 16. Reduction of mRNA Expression by Complexation with One or More Oligonucleotides

Oligonucleotides designed to reduce mRNA expression when the oligonucleotide is complexed with the mRNA were synthesized. These oligonucleotides were then tested for their ability to reduce mRNA expression relative the mRNA alone Oligonucleotides were annealed to a reporter mRNA (eGFPdeg) as described in Example 11, and mRNA expression was determined by the eGFPdeg IncuCyte Expression Assay described in Example 12.

Suppression of mRNA Expression by Oligonucleotides that Binds to the 5′UTR

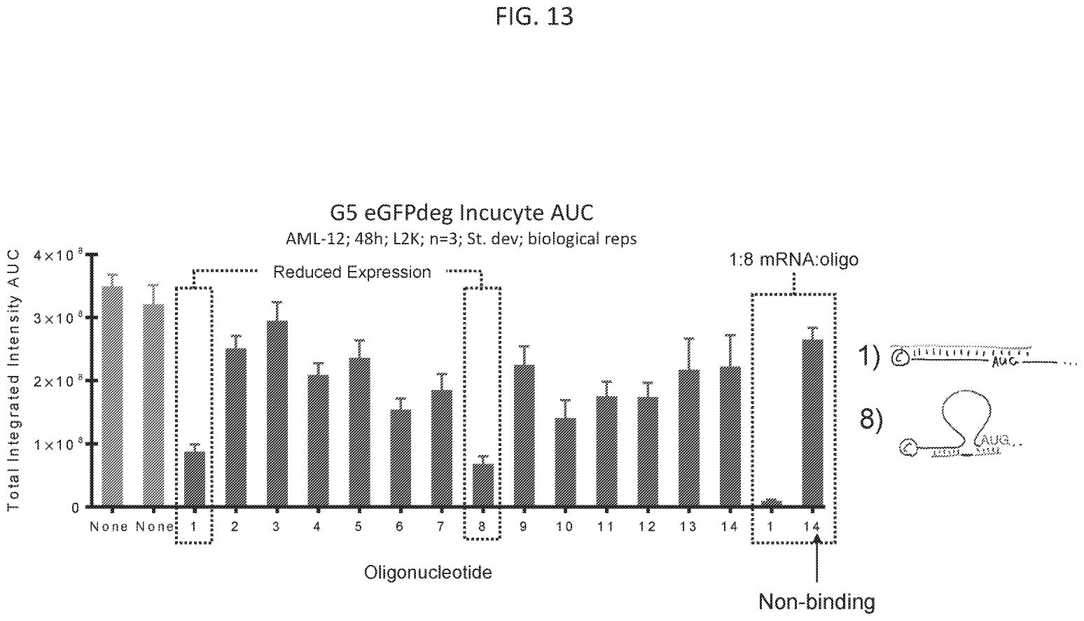

As shown in FIG. 13, an oligonucleotide bound to the 5′UTR of the eGFPdeg mRNA (oligonucleotide 1 of FIG. 13) was determined to suppress expression of eGFPdeg relative to the mRNA alone. Reduction of expression was found to be dependent on the concentration of oligonucleotide as an increased ratio of oligonucleotide (1:8 mRNA:oligo) resulted in an even greater suppression of mRNA expression.

Suppression of mRNA Expression by Oligonucleotides that Induce a Loop Conformation

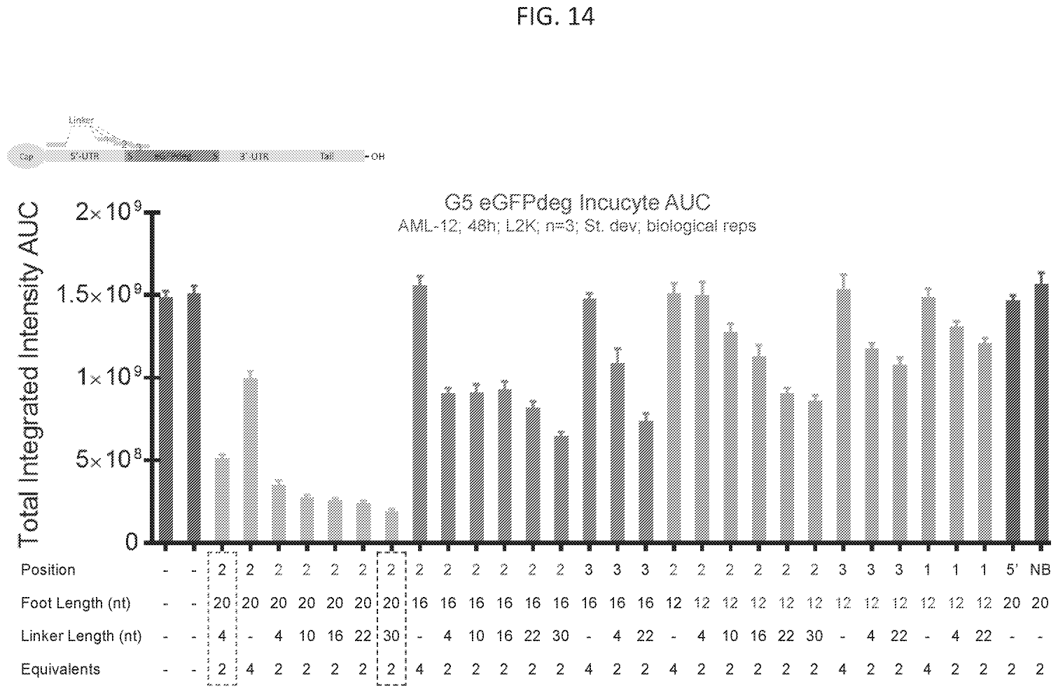

Also shown in FIG. 13 is the design and evaluation of a conjugate having the structure of A-L-B, where A is a first oligonucleotide, L is a linker (e.g., an oligonucleotide linker), and B is a second oligonucleotide, where A and B each include a region of linked nucleotides complimentary to a different portion of the sequence of an mRNA (oligonucleotide 8 of FIG. 13). Binding of this oligonucleotide to the mRNA is expected to induce a loop conformation in the mRNA. Binding of the oligonucleotide was determined to suppress expression of eGFPdeg relative to the mRNA alone. Additional conjugates having the structure of A-L-B were designed and synthesized to evaluate the effect of the length of each of A, L, and B, and the position of the conjugate on the mRNA on the suppression of mRNA expression. The corresponding results are provided in FIG. 14.

### Example 17. Induction of mRNA Geometries and Compaction as Determined by Fluorescence Resonance Energy Transfer (FRET)

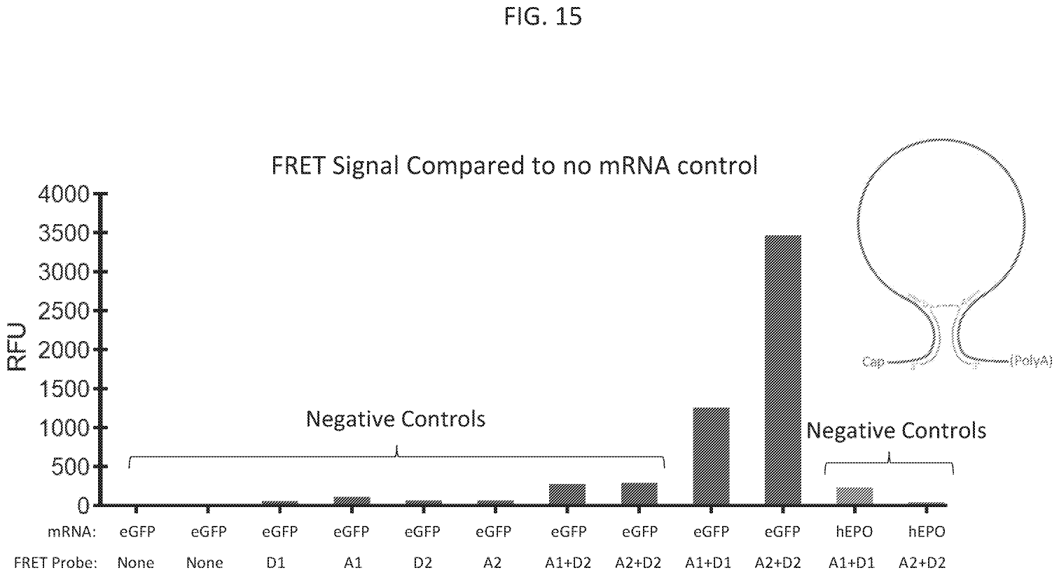

As described in Example 16, a conjugate was designed and synthesized having the structure of A-L-B, where A is a first oligonucleotide, L is a linker (e.g., an oligonucleotide linker), and B is a second oligonucleotide, where A and B each include a region of linked nucleotides complimentary to a different portion of the sequence of an mRNA. Binding of this conjugate to the mRNA was expected to induce a loop conformation in the mRNA. Compaction of the mRNA by induction of a loop conformation in the mRNA was confirmed by FRET (FIG. 15). Oligos were annealed as described in Example 11. FRET was measured by fluorescence with excitation and emission at 550 nm and 700 nm, respectively.

### Example 18. Effect of Oligonucleotide-Induced Compaction on mRNA Expression and Integrity

Multiple conjugates (e.g., 1, 2, 3, 4, or 5) were annealed to an mRNA, where each of the conjugates had the structure of A-L-B, where A is a first oligonucleotide, L is a linker (e.g., an oligonucleotide linker), and B is a second oligonucleotide, where A and B each include a region of linked nucleotides complimentary to a different portion of the sequence of an mRNA. Each set of multiple conjugates was designed to induce compaction of the mRNA, when bound to the mRNA. Schematics depicting mRNA compaction induced by binding of an mRNA to multiple conjugates having the structure of A-L-B are depicted in FIG. 16.

Effect of Oligonucleotide-Induced Compaction on mRNA Expression

The effect of conjugate-induced mRNA compaction on the expression of a reporter mRNA (eGFPdeg) was determined. Conjugates having the structure A-L-B were annealed to a reporter mRNA (eGFPdeg) as described in Example 11, and mRNA expression was determined by the eGFPdeg IncuCyte Expression Assay described in Example 12. The resulting expression data is provided in FIG. 16.

Effect of Oligonucleotide-Induced Compaction on mRNA Serum Half-Life

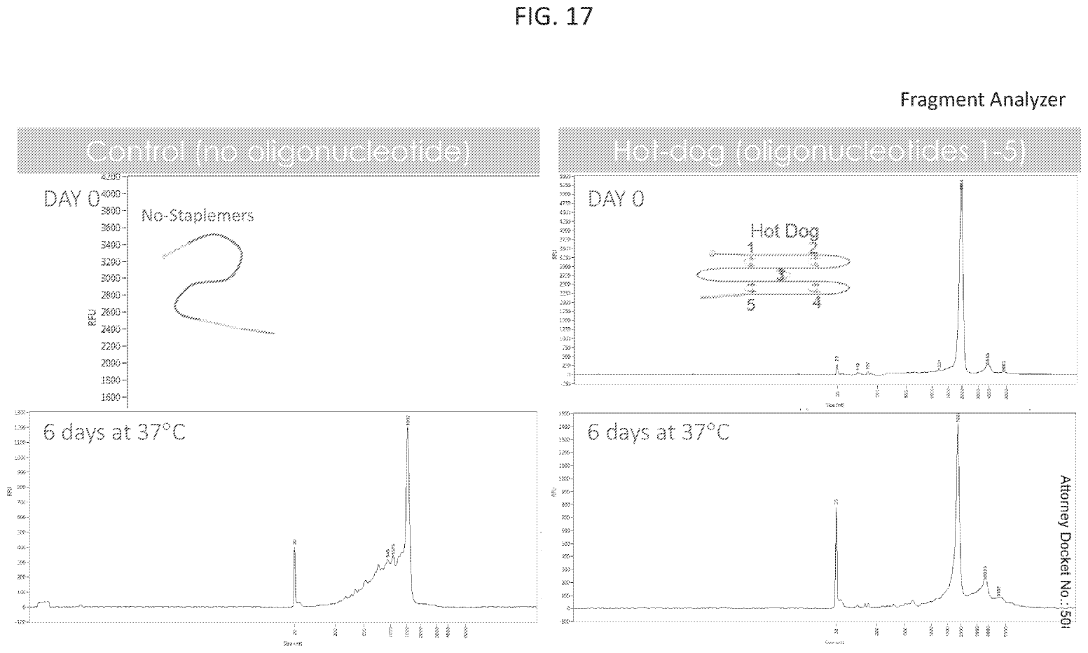

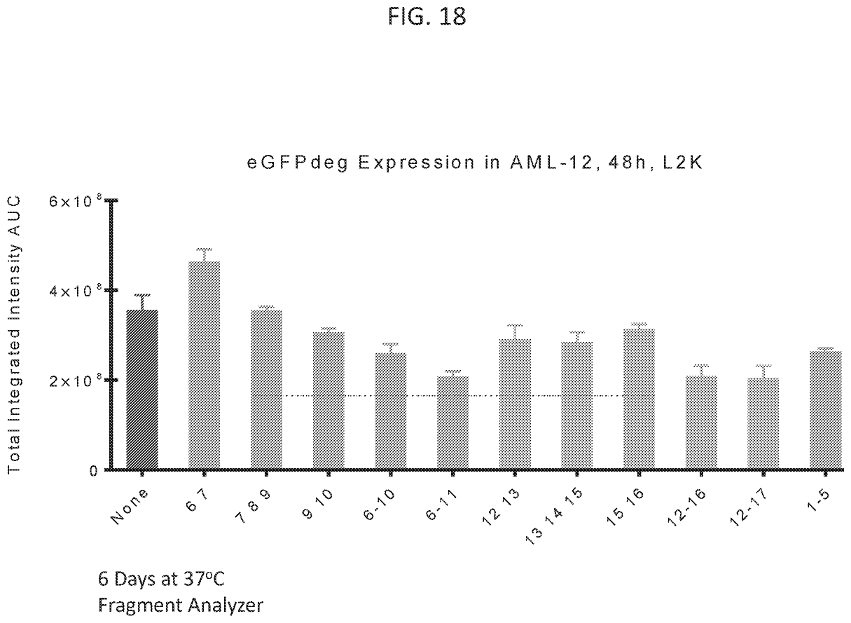

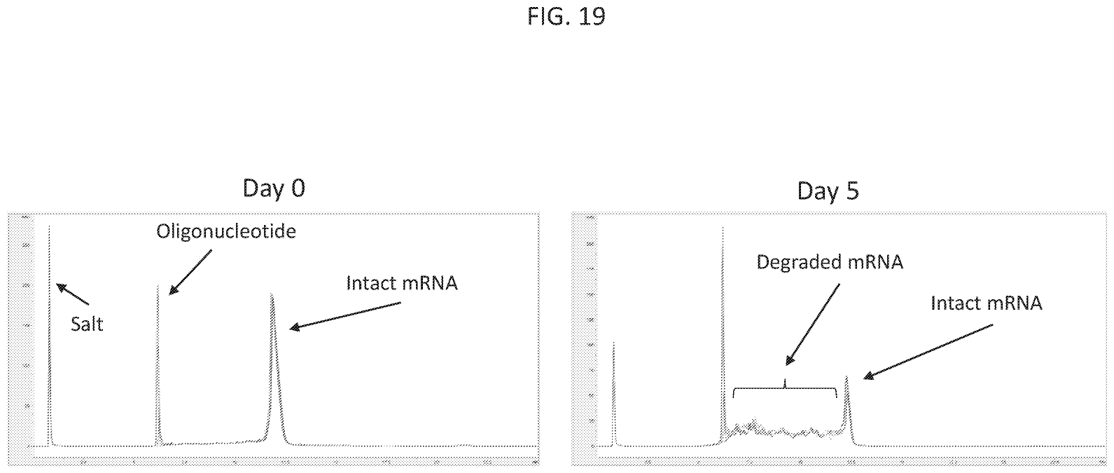

The effect of conjugate-induced mRNA compaction on the serum half-life of a reporter mRNA (eGFPdeg) was determined. Conjugates having the structure A-L-B were annealed to a reporter mRNA (eGFPdeg) as described in Example 11, and serum half-life was determined as described in Example 15. The compacted mRNA bound to multiple conjugates was incubated at 37° C. for 6 days and the resulting mRNA integrity data is provided in FIGS. 17-19.

### Example 19. Physical Co-Localization of Multiple mRNAs by Oligonucleotide Tethering

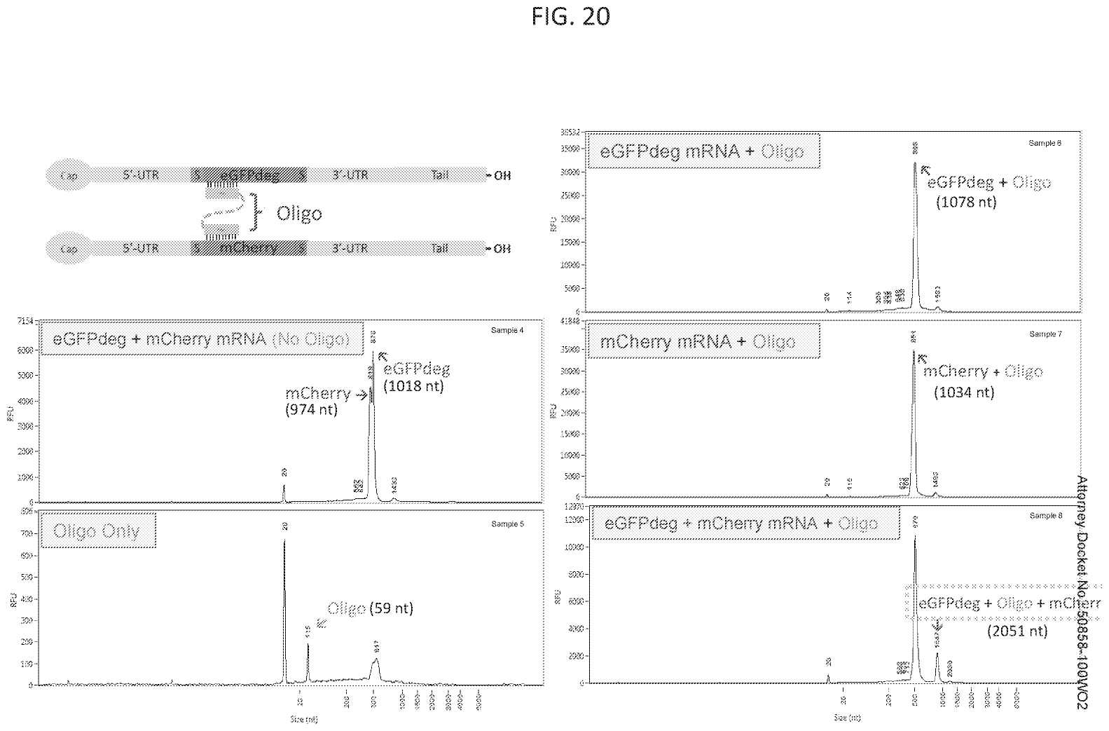

A conjugate that binds two separate mRNAs (eGFPdeg and mCherry) was designed and synthesized. The conjugates had the structure A-L-B, where A is a first oligonucleotide including a region of linked nucleotides complimentary to a portion of the sequence of a first mRNA (eGFPdeg), L is a linker (e.g., and oligonucleotide linker), and B is a second oligonucleotide including a region of linked nucleotides complimentary to a portion of the sequence of the second mRNA (mCherry). The conjugate was annealed to the mRNAs and binding was determined by size exclusion chromatography as described in Example 11. Complexation of the conjugate to eGFPdeg and mCherry mRNA is shown in FIG. 20. Samples were tested for integrity by Fragment Analyzer by Advanced Analytical (Ankeny, IA) using manufacturer protocols for long RNAs. The conjugate designed to bind two separate mRNAs was demonstrated to do so, causing the physical co-localization of these mRNAs.

### Example 20. Association and Expression of Cholesterol-Conjugated Oligonucleotides with mRNA

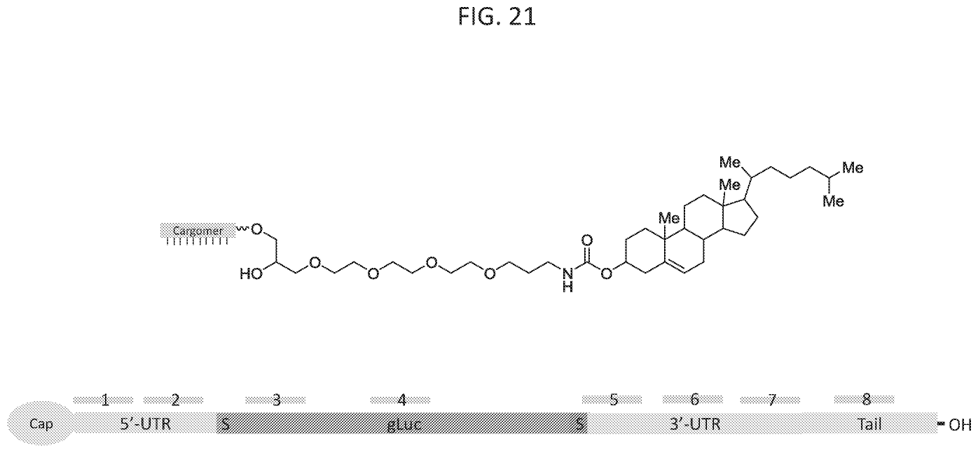

Conjugates were designed and synthesized, where the conjugate included an oligonucleotide having a region of linked nucleotides complimentary to a portion of the sequence of an mRNA, and where the oligonucleotide was covalently conjugated, through a triethylene glycol (TEG) linker, to a cholesterol moiety. The cholesterol-oligonucleotide conjugates were obtained from Integrated DNA Technologies (Skokie, II). Eight cholesterol-oligonucleotide which bound eight different regions of a reporter mRNA (gLuc) were designed and synthesized, as depicted in FIG. 21.

Association of Cholesterol-Oligonucleotides Conjugates with mRNA

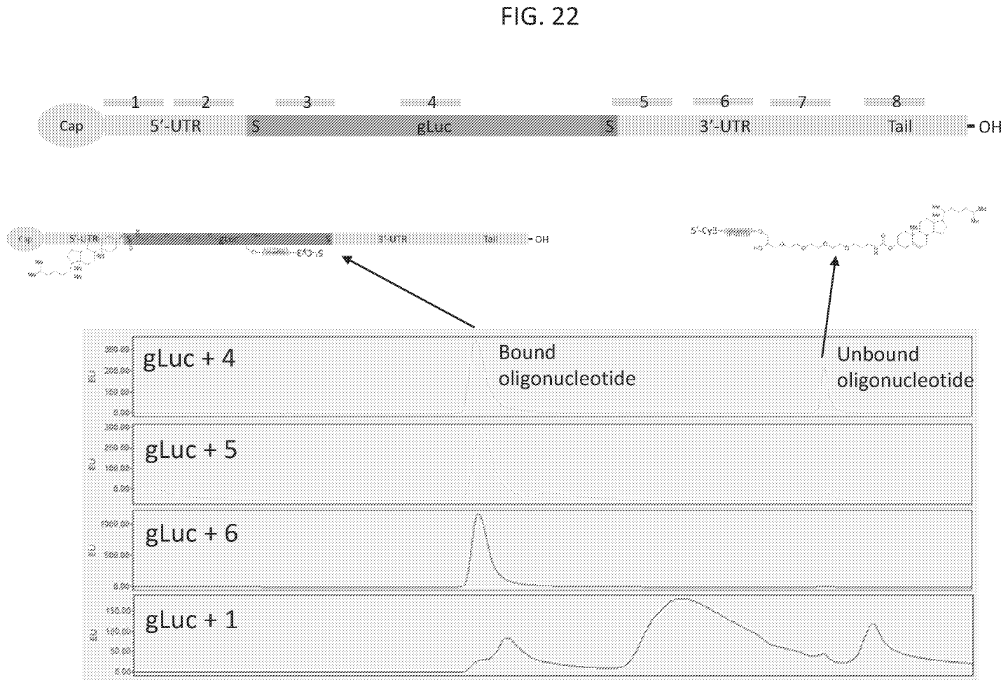

The ability of an oligonucleotide conjugated via a TEG linker to cholesterol to associate with an mRNA was determined. The cholesterol-oligonucleotide conjugate was annealed to a gLuc mRNA as described below. Size exclusion chromatography was used to evaluate the level of association as described in Example 11, the results of which are shown in FIG. 22.

Expression of mRNA Bound to One or More Cholesterol-Oligonucleotide Conjugates

Cholesterol-oligonucleotide conjugates having different locations of sequence complementarity to a gLuc mRNA were annealed to the gLuc mRNA and evaluated for their effect on mRNA expression. Additionally, combinations of multiple (2, 3, 4, 5, 6, 7, and 8) cholesterol-oligonucleotide conjugates having different locations of sequence complementarity to the gLuc mRNA were annealed to the gLuc mRNA and evaluated for their effect on mRNA expression. Annealing was performed in a 10:1 conjugate:mRNA molar ratio, as described below, and quantification of expression of gLuc was performed by the IncuCyte Expression assay described in Example 12.

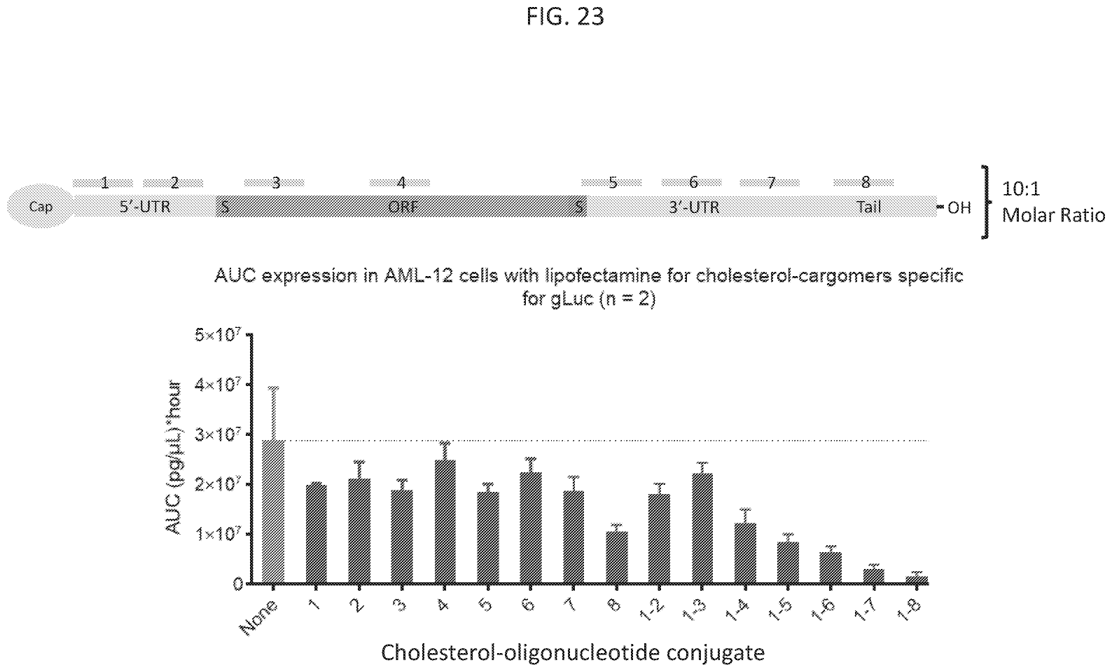

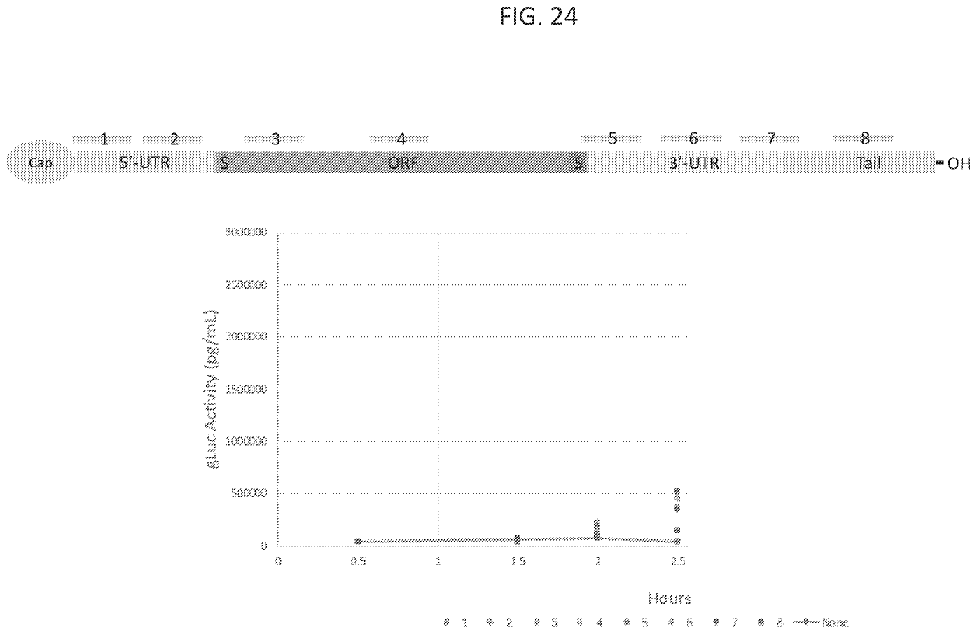

Expression levels show some degree of location-dependence. Expression was observed with all conjugates tested. Expression was also observed with multiple conjugates; in particular, up to 3 conjugates were well-tolerated. Expression data is provided in FIGS. 23-24.

Method: Annealing

Annealing of oligonucleotides and mRNA was performed in buffer containing 25 mM potassium chloride edta (Ambion, Waltham, Mass.) and 25 μM Ethylenediaminetetraacetic acid (Ambion, Waltham, Mass.). Oligonucleotides and mRNA were combined in the desired buffer and then heated to 70° C. before cooling at a rate of 1° C./second to a temperature of 25° C.

### Example 21. Reduction in Induced Innate Immune Response by Complexation of Cholesterol-Conjugated Oligonucleotides to mRNA

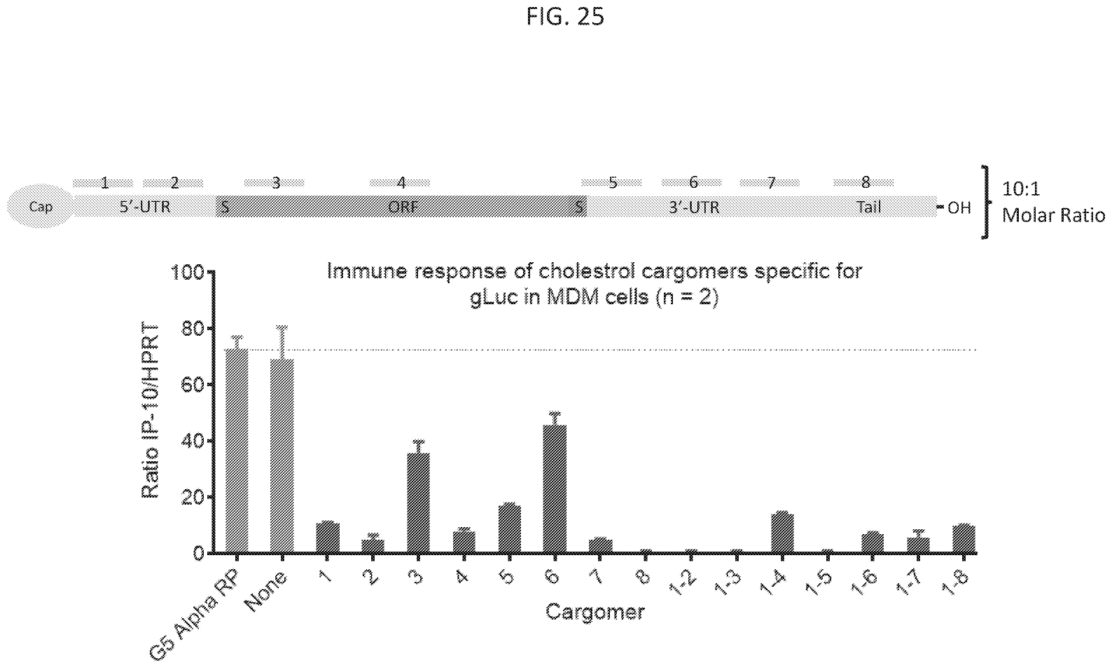

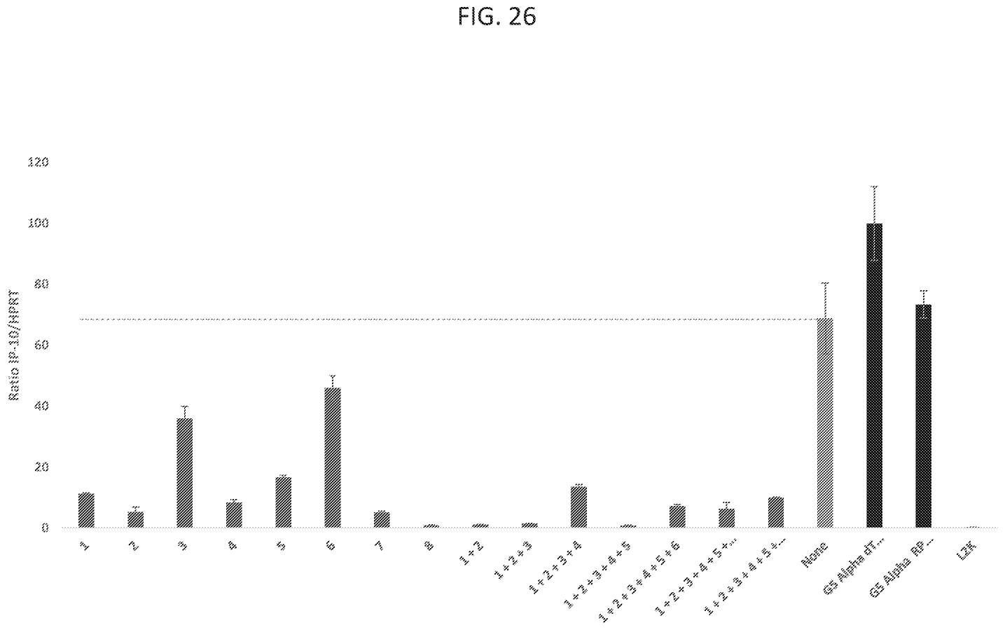

mRNA bound to one or more cholesterol-oligonucleotide conjugates was evaluated for its ability to activate the innate immune response in monocyte-derived macrophage (MDM) cells (measured by the ratio of IP-10/HPRT, as described below), as compared to the mRNA alone. Cholesterol-oligonucleotide conjugates having sequence complementarity to different portions of a reporter mRNA (gLuc) were evaluated. Additionally, combinations of multiple (2, 3, 4, 5, 6, 7, and 8) cholesterol-oligonucleotide conjugates having sequence complementarity to different portions of a reporter mRNA (gLuc) were evaluated. Complexation of the mRNA with one or more cholesterol-conjugated oligonucleotides significantly reduced the immune response observed in MDM cells, relative to mRNA alone. Reduction of the immune response was observed when annealing was performed at a 10:1 conjugate:mRNA molar ratio (FIG. 25) and at a 1:1 molar ratio (FIG. 26).

Monocyte-Derived Macrophage (MDM) Immune Response

Spleens were removed and mechanically homogenized via passage through a 70-micron filter, then washed with PBS+2% fetal bovine serum. ACK lysis buffer (Gibco, catalogue #A10492-01) was used to lyse red blood cells. Cells were transferred to a 96-well plate for staining and washed with PBS+2% fetal bovine serum. Cells were stained for 20 minutes on ice with the following antibodies: anti-CD3 efluor450 (eBioscience, catalogue #48-0031-82), anti-CD19 Alexafluor 700 (Invitrogen, catalogue #56-0193-82), anti-CD69 APC (BioLegend, catalogue #104514), and anti-CD86 PEcy5 (Invitrogen, catalogue #15-0862-82). Cells were washed three times with PBS+2% fetal bovine serum, then analyzed on an LSR Fortessa flow cytometer.

### Example 22. 3′ Stabilization of mRNA for Increased Protein Expression

Annealing oligonucleotides with complex secondary structures at the 3′end of mRNA (e.g., the 3′end of the poly(A) tail of mRNA) increases nuclease resistance and half-life of mRNA resulting in increased protein expression.

3′ Tailed Triple Helix

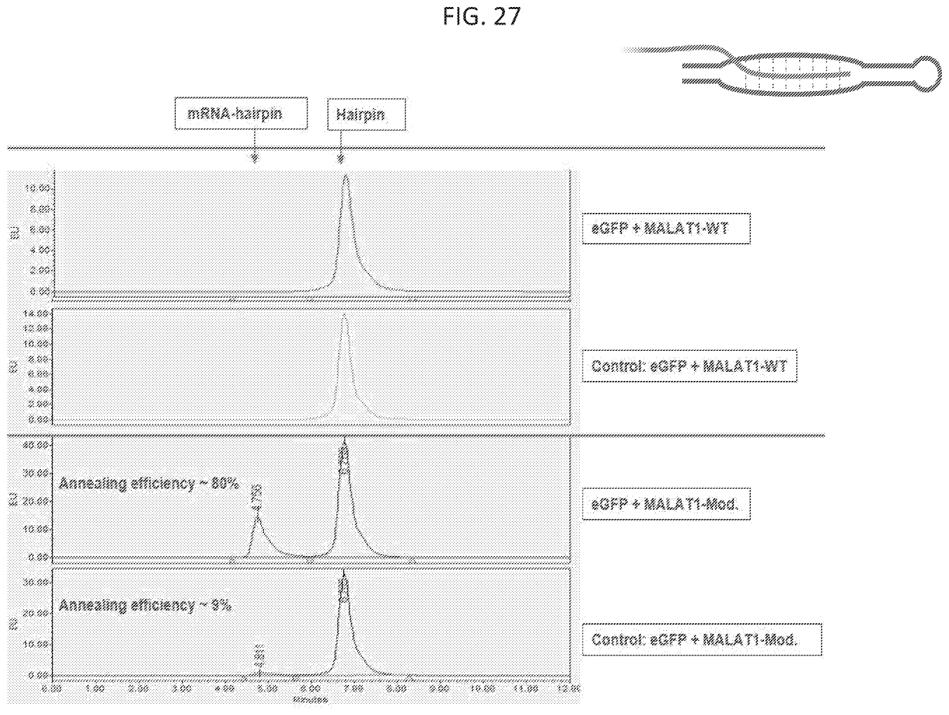

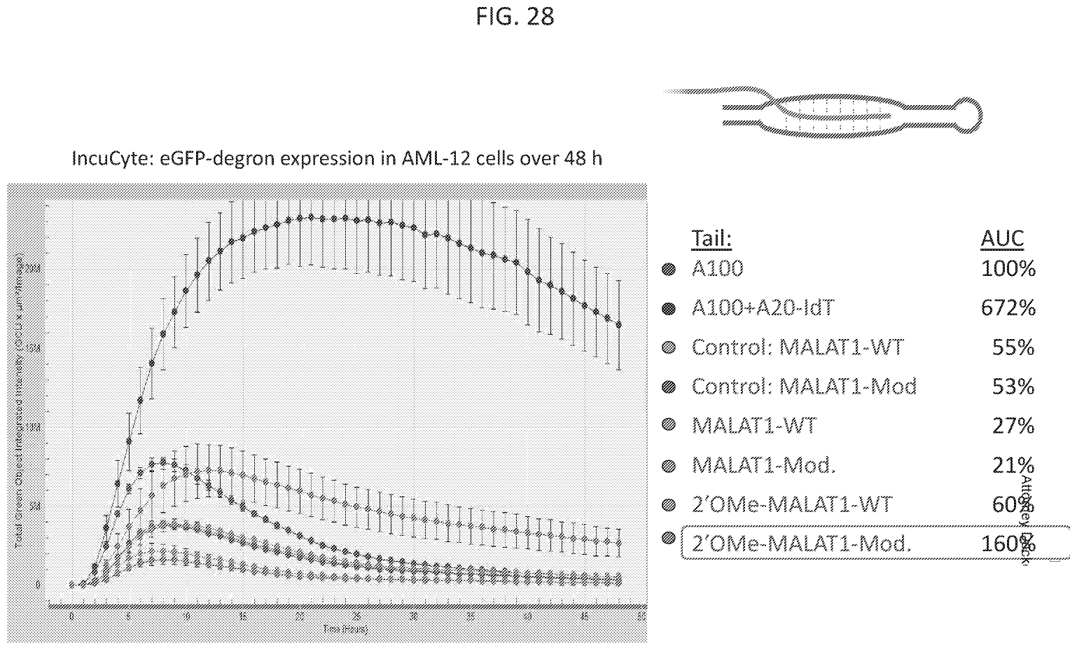

Oligonucleotides (e.g. oligonucleotides having a stem-loop structure) were designed and synthesized, where the oligonucleotide binds to the 3′ end of an mRNA (eGFP) and, upon binding, forms a triple helix structure with a region of nucleotides at the 3′ end of the mRNA. Specificity to 3′ end was achieved by complementarity between oligonucleotide and last 12 bases at the end of the mRNA. The oligonucleotide was annealed to eGFP mRNA at a ratio of 3:1 oligonucleotide to mRNA. Size exclusion chromatography was used to evaluate the level of association, the results of which are shown in FIG. 27. The level of mRNA expression was determined by the IncuCyte Expression Assay described in Example 12. The resulting expression data is provided in FIG. 28.

3′ Stem-Loop

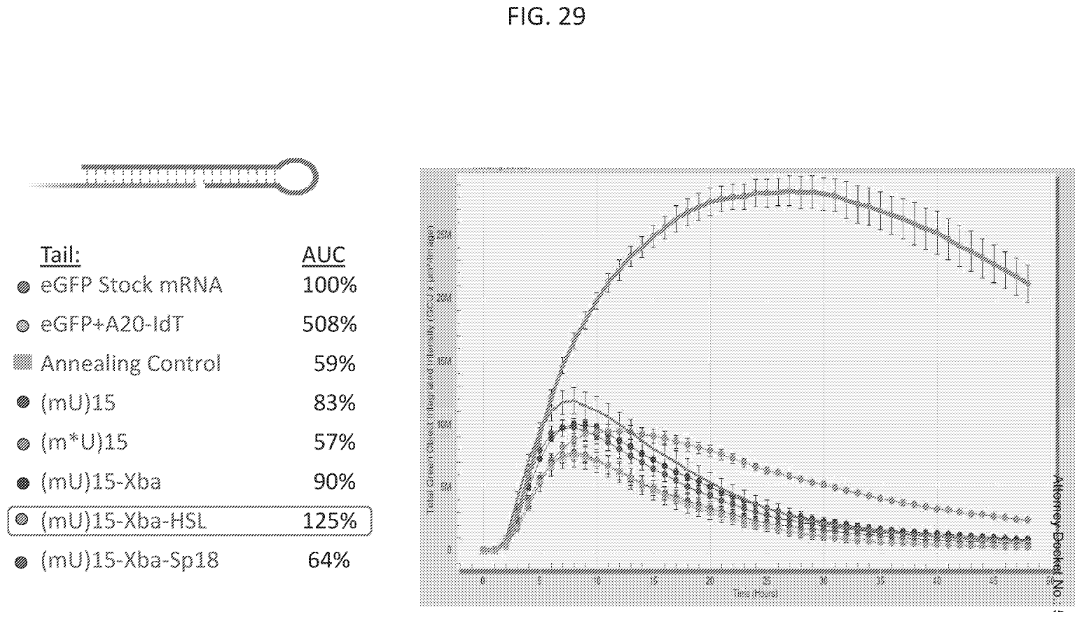

Oligonucleotides (e.g. oligonucleotides having a stem-loop structure) were designed and synthesized, where the oligonucleotide binds to the 3′ end of an mRNA (eGFP) and, upon binding, forms a stem-loop with a region of nucleotides at the 3′ end of the mRNA. Specificity to 3′ end was achieved by a poly(U) stretch in the oligonucleotide followed by 5 bases complimentary to Xba-I site, followed by a stem-loop. The oligonucleotide was annealed to eGFP mRNA at a ratio of 10:1 oligonucleotide to mRNA. The level of mRNA expression was determined by the IncuCyte Expression Assay described in Example 12. The resulting expression data is provided in FIG. 29.

### Other Embodiments

While the invention has been described in connection with specific embodiments thereof, it will be understood that it is capable of further modifications and this application is intended to cover any variations, uses, or adaptations of the invention following, in general, the principles of the invention and including such departures from the invention that come within known or customary practice within the art to which the invention pertains and may be applied to the essential features hereinbefore set forth, and follows in the scope of the claims. Other embodiments are within the claims.

1. A composition comprising:
(a) an mRNA encoding a polypeptide comprising:
(i) a 5′-cap structure;
(ii) a 5′-untranslated region (5′-UTR);
(iii) an open reading frame encoding the polypeptide;
(iv) a 3′-untranslated region (3′-UTR); and
(v) a poly-A region; and
(b) three or more oligonucleotides, wherein each oligonucleotide comprises a region of linked nucleotides complimentary to a different portion of the sequence of the mRNA.

2. The composition of claim 1, wherein:
a) the three or more oligonucleotides comprise at least three and no more than ten oligonucleotides;
b) the three or more oligonucleotides comprise at least ten and no more than fifty oligonucleotides;
c) the three or more oligonucleotides collectively comprise regions of linked nucleotides complementary to 10% or more of the sequence of the mRNA;
d) the three or more oligonucleotides each comprise between 6 and 100 nucleotides;
e) the three or more oligonucleotides comprise a region of linked nucleotides complementary to a portion of a sequence of the mRNA, wherein the region of linked nucleotides is at least 5 nucleotides;
f) the three or more oligonucleotides comprise at least one 2′-OMe nucleotide, 2′-MOE nucleotide, 2′-F nucleotide, 2′-NH₂ nucleotide, FANA nucleotide, LNA nucleotide, 4′-S nucleotide, TNA nucleotide, or PNA nucleotide;
g) at least one of the three or more oligonucleotides comprise at least on 2′-OMe nucleotide;
h) at least one of the three or more oligonucleotides comprise a region of linked nucleotides complementary to a portion of the sequence of the 5′-UTR or the 3′-UTR;
i) at least one of the three or more oligonucleotides comprise a region of linked nucleotides complementary to a portion of the sequence of the start codon;
j) the mRNA is hybridized to each of the three or more oligonucleotides; and/or
k) at least one of the three or more oligonucleotides is conjugated to a moiety selected from a sterol, a polyethylene glycol, a polylactic acid, a sugar, a toll-like receptor antagonist, or an endosomal escape peptide.

3-4. (canceled)

5. The composition of claim 2, wherein the three or more oligonucleotides collectively comprise regions of linked nucleotides complementary to 50% or more of the sequence of the mRNA.

6-18. (canceled)

19. The composition of claim 2, wherein the moiety is a sterol.

20. The composition of claim 19, wherein the sterol is cholesterol.

21-22. (canceled)

23. The composition of claim 1, wherein the composition is associated with a lipid nanoparticle.

24. A pharmaceutical composition comprising the composition of claim 1 and a pharmaceutically-acceptable excipient.

25. A method of increasing gene expression in a cell, the method comprising delivering to a cell the composition of claim 1.

26-31. (canceled)

32. The composition of claim 1, wherein the conjugate comprising an oligonucleotide comprising a region of linked nucleotides complimentary to a portion of the sequence of the mRNA oligonucleotide;
a) comprises at least 6 and no more than 100 nucleotides;
b) comprises at least one 2′-OMe nucleotide, 2′-MOE nucleotide, 2′-F nucleotide, 2′-NH₂ nucleotide, FANA nucleotide, LNA nucleotide, 4′-S nucleotide, TNA nucleotide, or PNA nucleotide;
c) comprises at least one 2′-OMe nucleotide;
d) consists of 2′-OMe nucleotides;
e) comprises a region of linked nucleotides complementary to a portion of the sequence of the 5′-UTR, the 3′-UTR, the open reading frame, the start codon, the stop codon, or poly-A region of the mRNA;
f) is hybridized to the mRNA;
g) comprises two or more sterol moieties; and/or
h) further comprises a second conjugate comprising a region of linked nucleotides complimentary to at least a second portion of the sequence of the mRNA.

33-94. (canceled)

95. A composition comprising:
(a) a first mRNA encoding a polypeptide comprising:
(i) a 5′-cap structure;
(ii) a 5′-UTR;
(iii) an open reading frame encoding the polypeptide;
(iv) a 3′-untranslated region (3′-UTR); and
(v) a poly-A region; and
(b) a second mRNA encoding a polypeptide comprising:
(i) a 5′-cap structure;
(ii) a 5′-UTR;
(iii) an open reading frame encoding the polypeptide;
(iv) a 3′-UTR; and
(v) a poly-A region; and
(c) a conjugate comprising the structure:
A-L-B
wherein A is a first oligonucleotide comprising a region of linked nucleotides complimentary to a portion of the sequence of the first mRNA, L is a linker, and B is a second oligonucleotide comprising a region of linked nucleotides complimentary to a portion of the sequence of the second mRNA.

96. The composition of claim 95, wherein:
a) L is an oligonucleotide linker;
b) L comprises a miRNA binding site;
c) L comprises an endonuclease binding site;
d) A and B each independently comprise at least 6 and no more than 100 nucleotides;
e) A and/or B comprises at least one 2′-OMe nucleotide, 2′-MOE nucleotide, 2′-F nucleotide, 2′-NH₂ nucleotide, FANA nucleotide, LNA nucleotide, 4′-S nucleotide, TNA nucleotide, or PNA nucleotide;
f) A comprises a region of linked nucleotides complementary to a portion of the sequence of the 5′-UTR, the 3′-UTR, the open reading frame, the start codon, the stop codon, or poly-A region of the mRNA;
g) B comprises a region of linked nucleotides complementary to a portion of the sequence of the 5′-UTR, the 3′-UTR, the open reading frame, the start codon, the stop codon, or poly-A region of the mRNA;
h) the first mRNA is hybridized to A and the second mRNA is hybridized to B; and/or
i) the conjugate further includes a moiety selected from a sterol, a polyethylene glycol, a polylactic acid, a sugar, a toll-like receptor antagonist, or an endosomal escape peptide.

97-110. (canceled)

111. The composition of claim 95, wherein the composition further comprises:
(d) a third mRNA encoding a polypeptide comprising:
(i) a 5′-cap structure;
(ii) a 5′-UTR;
(iii) an open reading frame encoding the polypeptide;
(iv) a 3′-UTR; and
(v) a poly-A region; and
(c) a second conjugate comprising the structure:
C-L-D
wherein C is a first oligonucleotide comprising a region of linked nucleotides complimentary to a portion of the sequence of the first or the second mRNA, L is a linker, and D is a second oligonucleotide comprising a region of linked nucleotides complimentary to a portion of the sequence of the third mRNA.

112. The composition of claim 95, wherein the composition is associated with a lipid nanoparticle.

113. A pharmaceutical composition comprising the composition of claim 95 and a pharmaceutically-acceptable excipient.

114-120. (canceled)

121. The composition of claim 1, wherein:
a) binding of the oligonucleotide that includes a stem-loop structure to the mRNA produces a triple helix structure or a stem-loop structure at the 3′ terminus of the mRNA;
b) the oligonucleotide that includes a stem-loop structure comprises between 10 and 200 nucleotides;
c) the portion of the sequence of the mRNA comprising the 3′-terminus of the mRNA comprises between 6 and 100 nucleotides;
d) the oligonucleotide that includes a stem-loop structure comprises at least one 2′-OMe nucleotide, 2′-MOE nucleotide, 2′-F nucleotide, 2′-NH₂ nucleotide, FANA nucleotide, LNA nucleotide, 4′-S nucleotide, TNA nucleotide, or PNA nucleotide; and/or
(e) the oligonucleotide further comprises a moiety selected from a sterol, a polyethylene glycol, a polylactic acid, a sugar, a toll-like receptor antagonist, or an endosomal escape peptide.

122-140. (canceled)

141. A double-stranded RNA including (a) a first strand having:
(i) a 5′-cap structure;
(ii) a 5′-UTR;
(iii) an open reading frame encoding the polypeptide;
(iv) a 3′-untranslated region (3′-UTR); and
(v) a poly-A region; and
(b) a second strand including one or more oligonucleotides including two regions of linked nucleotides complementary to non-contiguous portions of the sequence of the mRNA.

142. The double-stranded RNA of claim 141, wherein:
a) the double-stranded RNA is more compact than a corresponding RNA including only the first strand;
b) the double-stranded RNA when administered to a cell, has a longer half-life compared to a corresponding RNA including only the first strand;
c) the double-stranded RNA, when administered to a cell in the absence of a lipid nanoparticle results in greater expression compared to a corresponding RNA including only the first strand;
d) the double-stranded RNA, when contacted with an LNP, has greater loading compared to a corresponding RNA including only the first strand;
e) the oligonucleotide comprises at least one 2′-OMe nucleotide, 2′-MOE nucleotide, 2′-F nucleotide, 2′-NH₂ nucleotide, FANA nucleotide, LNA nucleotide, 4′-S nucleotide, TNA nucleotide, or PNA nucleotide;
f) the oligonucleotide further comprises a moiety selected from a sterol, a polyethylene glycol, a polylactic acid, a sugar, a toll-like receptor antagonist, or an endosomal escape peptide.

143-151. (canceled)

152. The composition of claim 141, wherein the composition is associated with a lipid nanoparticle.

153-154. (canceled)

155. A pharmaceutical composition comprising the composition of claim 141 and a pharmaceutically-acceptable excipient.

156-161. (canceled)


SEQUENCE LISTING (14 sequences)

SEQ ID NO: 1 - DNA, 120 bp, Artificial Sequence
  Synthetic Construct
tttttttttttttttttttttttttttttttttttttttttttttttttttttttttttttttttttttttttttttttttttttttttttttttttttttttttttttttttttttttt

SEQ ID NO: 2 - DNA, 120 bp, Artificial Sequence
  Synthetic Construct
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 3 - DNA, 165 bp, Artificial Sequence
  Synthetic Construct
  misc_feature (151)..(165): n is a or absent
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaannnnnnnnnnnnnnn

SEQ ID NO: 4 - DNA, 155 bp, Artificial Sequence
  Synthetic Construct
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 5 - DNA, 156 bp, Artificial Sequence
  Synthetic Construct
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 6 - DNA, 157 bp, Artificial Sequence
  Synthetic Construct
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 7 - DNA, 158 bp, Artificial Sequence
  Synthetic Construct
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 8 - DNA, 159 bp, Artificial Sequence
  Synthetic Construct
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 9 - DNA, 160 bp, Artificial Sequence
  Synthetic Construct
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 10 - DNA, 161 bp, Artificial Sequence
  Synthetic Construct
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 11 - DNA, 162 bp, Artificial Sequence
  Synthetic Construct
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 12 - DNA, 163 bp, Artificial Sequence
  Synthetic Construct
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 13 - DNA, 164 bp, Artificial Sequence
  Synthetic Construct
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 14 - DNA, 165 bp, Artificial Sequence
  Synthetic Construct
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa